(19) United States
(12) Patent Application Publication
(10) Pub. No.: US 2026/0028389 A1
(43) Pub. Date: Jan. 29, 2026

(54) FUSION PROTEIN INHIBITORS OF KLK5

(71) Applicant: BioCryst Pharmaceuticals, Inc., Durham, NC (US)

(72) Inventors: Yarlagadda S. Babu, Birmingham, AL (US); Xilin Chen, Alpharetta, GA (US)

(21) Appl. No.: 18/996,497

(22) PCT Filed: Jul. 19, 2023
(86) PCT No.: PCT/US2023/028123

§ 371 Date: Jan. 17, 2025

Related U.S. Application Data
(60) Provisional application No. 63/390,780, filed on Jul. 20, 2022.

(51) Int. Cl. C07K 14/81; A61K 38/00

(52) U.S. Cl. CPC C07K 14/811; A61K 38/00; C07K 2319/30; C07K 2319/50

(57) ABSTRACT

Disclosed herein are polypeptides that are useful for, among other things, inhibiting or suppressing the activity of a kallikrein-related protease and therefore treating diseases and conditions involving dysregulated kallikrein-related protease activity. Pharmaceutical compositions and therapeutic methods using the polypeptides and pharmaceutical compositions are also provided.

### RELATED APPLICATION

This application claims the benefit of priority to U.S. Provisional Patent Application Ser. No. 63/390,780, filed Jul. 20, 2022.

### BACKGROUND

Tissue kallikreins (KLKs) are a family of 15 trypsin- and chymotrypsin-like serine proteases. KLKs are secreted as pro-enzymes, requiring the removal of terminal peptide portions through specific amino-terminal proteolysis for activation. Some KLKs are reliant on activation by other KLKs or other proteases, while some KLKs, such as KLK5, are capable of self-activation. As such, KLKs function through proteolytic cascades in the body. KLKs are widely expressed in a diverse range of tissues including the kidney, brain, respiratory, gastrointestinal, epidermis, and reproductive tracts. KLKs regulate a number of essential physiological functions generally in a cell-specific manner, including modulating immunity, inflammation, and carcinogenesis.

In addition to other roles, KLKs play an important role in the epidermis. KLK5 and KLK7 degrade desmosomal proteins, a group of proteins responsible for the structural integrity of the epidermis. For example, the shedding of old cells from the skin surface and maintaining a healthy epidermis requires the degradation of desmosomes, a process known as desquamation (Simon et al. 2001; Caubet et al. 2004). As such, KLKs play a role in maintaining the integrity of the skin barrier, and in preventing desquamation and inflammation. KLK5 is an important regulator of proteolytic activity in the epidermis, as KLK5 can activate not only its own pro-enzyme but also many other KLK family pro-enzymes, and has been shown to activate proKLK7 (Caubet et al. 2004; Brattsand et al. 2005; Yoon et al. 2007). KLK5 has also been shown to activate the metallo-proteases meprin-α and meprin-β, which are associated with basal keratinocyte proliferation and differentiation processes (Ohler et al. 2010). In addition, KLK5 and KLK14 can activate proteinase-activated receptor-2 (PAR 2), a G-protein-coupled receptor that is present on the membrane of many cell types including keratinocytes, and plays a regulatory role in the skin during inflammation (including production of pro-inflammatory cytokines leading to the generation of a pro-inflammatory environment, activation of Langerhans cells, and induction of pro-allergic Th2 cells), epidermal barrier function, and pruritus (Oikonomopoulou et al. 2006; Stefansson et al. 2008). KLKs, including KLK5, are also involved in the processing of cathelicidin antimicrobial peptides that kill microbes, and modify host immunity and cell growth responses (Lai and Gallo, 2009; Yamasaki et al. 2006; Eissa et al. 2011).

Several skin disorders are characterized by elevated protease activity (Komatsu et al. 2002, 2008; Descargues et al. 2006; Yamasaki et al. 2007; Cork et al. 2009). Increased KLK5 activity can be observed in skin samples from Rosacea patients who also show abnormally high levels of cathelicidins in their facial skin (Yamasaki et al. 2007). Netherton syndrome is a severe autosomal recessive disorder also associated with increased KLK5 activity that is characterized by congenital ichthyosis with defective cornification, a specific hair shaft defect (bamboo hair) and severe atopic manifestations including atopic dermatitis and hay fever (Netherton, 1958; Burns et al. 2004).

However, treatment of diseases and conditions related to increased activity of KLKs has mainly been non-specific and aimed at symptoms rather than the root cause of disease, and incurring unwanted side effects, such as atrophy after prolonged use of glucocorticoids. Accordingly, there exists a need to develop further KLK inhibitors, in particular KLK5 inhibitors, which have therapeutic potential in the treatment of numerous diseases and conditions.

### SUMMARY

In one aspect, the present disclosure provides a SPINK9 polypeptide, comprising a (e.g., at least one) mutant SPINK9 domain. In some embodiments, said at least one mutant SPINK9 domain comprises an amino acid sequence represented by Formula I: Ile Glu Cys Ala Lys Gln Thr Lys Gln Met Val Asp Cys Ser His Tyr Lys Lys Leu Pro Pro X₁ Gln X₂ X₃ X₄ Cys X₅ X₆ X₇ Tyr Asp Pro Ile Cys Gly Ser Asp Gly Lys Thr Tyr X₈ Asn Asp Cys Phe Phe Cys Ser Lys Val Lys Lys Thr Asp Gly Thr Leu Lys Phe Val His Phe Gly Lys Cys (SEQ ID NO: 3), wherein X₁-X₈ are as defined herein.

In some embodiments, said at least one mutant SPINK9 domain comprises an amino acid sequence represented by Formula III X₉ X₁₀ X₁₁ X₁₂ X₁₃ X₁₄ X₁₅ X₁₆ X₁₇ Met Val Asp Cys Ser His Tyr Lys Lys Leu Pro Pro X₁ Gln X₂ X₃ X₄ Cys X₅ X₆ X₇ Tyr Asp Pro Ile Cys Gly Ser Asp Gly Lys Thr Tyr X₈ Asn Asp Cys Phe Phe Cys Ser Lys Val Lys Lys Thr Asp Gly Thr Leu Lys Phe Val His Phe Gly Lys Cys (SEQ ID NO: 135), wherein X₁-X₁₇ are as defined herein.

In some embodiments, said at least one mutant SPINK9 domain comprises an amino acid sequence represented by Formula IV: X₉ X₁₀ X₁₁ Ala Lys Gln Thr Lys Gln Met Val Asp Cys Ser His Tyr Lys Lys Leu Pro Pro X₁ Gln X₂ X₃ X₄ Cys X₅ X₆ X₇ Tyr Asp Pro Ile Cys Gly Ser Asp Gly Lys Thr Tyr X₈ Asn Asp Cys Phe Phe Cys Ser Lys Val Lys Lys Thr Asp Gly Thr Leu Lys Phe Val His Phe Gly Lys Cys (SEQ ID NO: 136), wherein X₁-X₁₁ are as defined herein.

Preferably, the amino acid sequences represented by Formula I, Formula III, and Formula IV are not the wild type sequence of the SPINK9 domain (i.e., SEQ ID NO: 2).

In further aspects, the present disclosure provides compositions, including pharmaceutical compositions, comprising a SPINK9 polypeptide, genetic constructs, including nucleic acid sequences, encoding a SPINK9 polypeptide, host cells comprising such genetic constructs, and methods for treating diseases and conditions using said SPINK9 polypeptides and pharmaceutical compositions thereof. Such SPINK9 polypeptides may be used to inhibit one or more KLKs, such as KLK5.

### BRIEF DESCRIPTION OF THE DRAWINGS

FIG. 1 shows mean body weight of animals administered mutant a representative SPINK9 polypeptide.

FIG. 2 shows blood plasma concentration of a representative mutant SPINK9 polypeptide using a Fc+Fc ELISA method.

FIG. 3 shows blood plasma concentration of a representative mutant SPINK9 polypeptide using a KLK5+Fc ELISA method.

### DETAILED DESCRIPTION

KLKs and their specific inhibitors have a pivotal role in regulating proteolytic activity, particularly in the skin. Alterations of this equilibrium resulting in dysregulation of KLK expression and/or activity is implicated in a number of human diseases and conditions (Paliouras, M, Biol Chem, 2006, Vol 387, pages 643-652). In particular, the serine protease KLK5 and the serine protease inhibitor SPINK5 have each been shown to be involved in the regulation of epidermal desquamation. The SPINK5 gene codes for the 15-domain protein serine protease inhibitor Kazal-type 5 (SPINK5; also known as Lymphoepithelial Kazal type-related inhibitor, LEKTI).

SPINK5 is synthesized as three different high-molecular-weight precursors containing up to 15 inhibitory domains typified by a Kazal-like domain arrangement. The precursors are rapidly processed into shorter fragments and secreted extracellularly. Several of these shorter fragments have been shown to inhibit KLK5 activity. The inhibitory effect of wild-type SPINK5 and related KLKs is pH-dependent, with loss of KLK inhibition at acidic pH similar to that of the outer layers of the skin. Loss of function mutations in SPINK5 lead to the absence of KLK5 inhibitory activity and hyperactivation of KLK5.

The loss of function in mutations in SPINK5 and resulting increase in serine protease (e.g., KLK5) activity have been shown to be a causative factor in a variety of diseases, such as Netherton syndrome and autosomal recessive ichthyosis with hypotrichosis. For example, Netherton syndrome is associated with mutations in the SPINK5 gene indicating that serine proteases are indeed important for skin physiology (Chavanas et al. 2000). Netherton syndrome is characterized by congenital erythrodermic ichthyosis, hair defects, and atopic manifestations (Comel, 1949; Netherton, 1958; Chavanas et al., 2000). Severe complications are also associated with Netherton syndrome, including recurrent bacterial infections, hypernatremic dehydration, and failure to thrive.

Serine protease inhibitor Kazal-type 9 (SPINK9) is a 7.7 kDa serine protease inhibitor related to SPINK5 and is almost exclusively expressed in areas where the stratum corneum is thickened or where hyperkeratosis occurs, such as the upper layers of the palmo-plantar epidermis and in the clavi (Brattsand et al. 2009; Meyer-Hoffert et al. 2009). Unlike SPINK5, SPINK9 contains a single inhibitor domain, which has been shown to be a KLK5-specific inhibitor. Notably, two areas of the body not affected by congenital SPINK5 mutations in Netherton's syndrome are areas where SPINK9 is expressed, namely the soles of the feet and palm. Therefore, SPINK9 also plays a role in regulating skin physiology.

As disclosed herein, a SPINK9 polypeptide, and modifications and variants thereof, may be exploited for its interactions with KLK proteases (e.g., KLK5). Accordingly, provided herein are polypeptides, and compositions thereof, that target enzymes (e.g., proteases of the KLK family) associated with disease, particularly disorders of the skin and methods of treatment using such polypeptides and compositions thereof. Such polypeptides and compositions thereof are useful for treating or preventing diseases and conditions characterized by aberrant KLK activity (e.g., aberrant KLK5 activity).

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g., “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

The terms “administering,” “administer” or “administration of” as used herein refers to the introduction of an agent, including a therapeutic agent, to a subject and can be carried out using one of a variety of methods known to those skilled in the art. For example, administering can be performed, for example, intravenously, arterially, intradermally, intramuscularly, intraperitoneally, subcutaneously, ocularly, sublingually, orally (by ingestion), intranasally (by inhalation), intraspinally, intracerebrally, topically, and transdermally (by absorption). Administering can also be performed by rechargeable or biodegradable polymeric devices or other devices, e.g., patches and pumps, or formulations, which provide for the extended, slow or controlled release of the compound or agent. Administering can be performed, for example, once, a plurality of times, and/or over one or more extended periods.

The term “biologically active fragment” as used herein refers to a fragment of a polypeptide, which still contains a specific biological activity of said parent polypeptide. For example, in the context of the present disclosure, a “biologically active fragment” of SPINK9 is a fragment of SPINK9, including a mutant SPINK9 domain, that is able to inhibit or suppress the activity of a KLK (e.g., KLK5) as determined using the method described in Example 2 herein. In some embodiments, a “biologically active fragment” of SPINK9 is a fragment of SPINK9, including a mutant SPINK9 domain, that is able to inhibit or suppress the activity of a KLK (e.g., KLK5) by at least 50%, 55%, 60%, 65%, 70%, 75%, 80%, 85%, 90%, 95%, or greater as compared to wild type SPINK9 (e.g., SPINK9 having the sequence of SEQ ID NOS: 1 or 2).

The phrase “conjoint administration” or “conjointly administered” as used herein refers to any form of administration of two or more different agents such that the second agent is administered while the previously administered agent is still effective in the body (e.g., the two agents are simultaneously effective in the patient, which may include synergistic effects of the two agents). For example, the different agents can be administered either in the same formulation or in separate formulations, either concomitantly or sequentially. Thus, a patient who receives such treatment can benefit from a combined effect of different agents.

The phrase “consisting essentially of” as used herein to refer to chemical compounds and compositions means that specific further components can be present in such chemical compound or composition, namely those not materially affecting the essential characteristics of the compound or composition (e.g., inhibitory activity of a SPINK9 polypeptide or inhibitory activity of a mutant SPINK9 domain).

The term “inhibit” as used herein means decrease by an objectively measurable amount or extent. In various embodiments, “inhibit” means decrease by at least 5, 10, 20, 30, 40, 50, 60, 70, 80, 90, or 95 percent compared to a relevant control. In one embodiment, “inhibit” means decrease 100 percent, i.e., halt or eliminate.

The term “mutant” as used herein refers broadly to a polypeptide comprising one or more sequence changes compared to a reference polypeptide (e.g., a wild-type polypeptide) or a nucleic acid encoding said reference polypeptide. In some embodiments, a mutant polypeptide comprises an artificially introduced change in amino acid sequence (e.g., a change in amino acid sequence generated in the laboratory or other facility by human intervention). Such mutant polypeptides may contain a single change or multiple changes in amino acid sequence as compared to a reference polypeptide. Such amino acid changes include, but are not limited to, substitutions, insertions, and deletions. Guidance for substitutions, insertions, or deletions may be based on alignments of amino acid sequences of different variant proteins or proteins from different species.

The term “mutant SPINK9 domain” as used herein refers to a sequence in a polypeptide comprising one or more amino acid sequence changes compared to wild-type SPINK9 (e.g., SEQ ID NOS: 1 or 2). In some embodiments, a mutant SPINK9 domain sequence is at least 70% (e.g., at least 75%, at least 80%, at least 85%, at least 90%/o, at least 95%) identical to a wild-type SPINK9 sequence (e.g., SEQ ID NO: 1 or 2). In some embodiments, the sequence change is an artificially introduced sequence change. Such “mutant SPINK9 domain” may contain a single change in amino acid sequence or multiple changes in amino acid sequence as compared to wild-type SPINK9. For example, in some embodiments, a mutant SPINK9 domain sequence contains 1, 2, 3, 4, 5, 6, 7, 8, 9, or 10 amino acid sequence differences compared to a wild-type SPINK9 sequence.

The terms “patient,” “subject,” or “individual” are used herein are interchangeably herein and refer to either a human or a non-human animal. The term non-human animal includes, but is not limited to, mammals, such as humans, primates, livestock animals (including bovines, porcines, etc.), companion animals (e.g., canines, felines, etc.) and rodents (e.g., mice and rats). In one embodiment, a subject is human.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “pharmaceutically acceptable salt” or “salt” is used herein to refer to an acid addition salt or a basic addition salt, which is suitable for or compatible with the treatment of patients.

The terms “preventing” or “prevention” are art-recognized, and when used in relation to a disease (such as Netherton syndrome) or any other medical condition, is well understood in the art, and includes administration of a composition which reduces the frequency of, or delays the onset of, symptoms of a medical condition in a subject relative to a subject which does not receive the composition. Thus, prevention of Netherton syndrome includes, for example, reducing the number or severity of atopic manifestations of the disease in a population of subjects receiving a prophylactic treatment relative to an untreated control population, and/or delaying the appearance of atopic manifestations of the disease in a population of subjects receiving a prophylactic treatment relative to an untreated control population, e.g., by a statistically and/or clinically significant amount.

The terms “prodrug” or “pharmaceutically acceptable prodrug” as used herein refer to a compound that is metabolized, for example hydrolyzed or oxidized, in the host after administration to form an active compound (e.g., a compound of the present disclosure). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference in their entirety. The prodrugs of this disclosure are metabolized to produce a SPINK9 polypeptide. The present disclosure includes within its scope prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filler, diluent, excipient, solvent or material useful for formulating a SPINK9 polypeptide or other compound disclosed herein for medicinal or therapeutic use. Each carrier must be “acceptable” in the sense of being compatible with the other ingredients of the formulation and not injurious to the patient. Some examples of materials which can serve as pharmaceutically acceptable carriers include: (1) water, any and all solvents, dispersion media, diluents, or other liquid vehicles; (2) dispersion or suspension aids; (3) surfactants and surface active agents; (4) isotonic agents, thickening or emulsifying agents; (5) preservatives, antioxidants, chelating agents, solid binders, lubricants; (6) sugars, such as dextrans, lactose, glucose and sucrose; (7) starches, such as corn starch and potato starch; (8) cellulose, and its derivatives, such as sodium carboxymethyl cellulose, ethyl cellulose and cellulose acetate; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) buffering agents, such as magnesium hydroxide and aluminum hydroxide, isotonic saline, Ringer's solution, phosphate buffer solutions; (14) osmolality adjusting agents; (15) stabilizing agents, and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various carriers used in formulating pharmaceutical compositions and known techniques for the preparation thereof. Except insofar as any conventional carrier is incompatible with a substance or its derivatives, such as by producing any undesirable biological effect or otherwise interacting in a deleterious manner with any other component(s) of the composition, its use is contemplated to be within the scope of this present disclosure.

The term “sequence identity”, as used herein, refers to the sequence similarity between two peptide/polypeptide/protein molecules. When a position in both of the two compared sequences is occupied by the same amino acid monomer subunit, for example, then the molecules are identical at that position. Without being bound by any particular theory or methodology, the percent sequence identity between two sequences may be considered a function of the number of identical positions shared by the two sequences divided by the number of positions compared, and multiplied by 100. For example, if 6 of 10 of the positions in two sequences are the same, then the two sequences have 60% sequence identity. Generally, a comparison is made when two sequences are aligned to give maximal identical positions.

The term “SPINK9 polypeptide” as used herein refers to a polypeptide comprising a mutant SPINK9 domain and optionally an additional domain (e.g., a Fc portion). In certain embodiments, a “SPINK9 polypeptide” comprises more than 1 mutant SPINK9 domain (e.g., from 1 to 20, 1 to 15, 1 to 10, 1-5, 1-4, 1-3, or 2 mutant SPINK9 domains) and optionally an additional domain (e.g., a Fc portion). In some embodiments, the SPINK9 polypeptide can comprise multiple associated polypeptide chains (e.g., as a polypeptide dimer linked through a Fc portion or other dimerization domain). In some embodiments, each polypeptide chain in a SPINK9 polypeptide comprises a mutant SPINK9 domain.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated. The skilled worker can readily determine the effective amount for a given situation by routine experimentation.

The terms “treat”, “treating”, and “treatment” as well understood in the art, refer to taking steps to obtain beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

Amino acid residues may be referred to by their corresponding three letter code or by their corresponding one letter code. The various three letter and single letter codes for the 20 commonly used amino acids are provided below.

| | 3 Letter | 1 Letter | | 3 Letter | 1 Letter |
|---|---|---|---|---|---|
| Amino Acid | Code | Code | Amino Acid | Code | Code |
| Glycine | Gly | G | Proline | Pro | P |
| Alanine | Ala | A | Valine | Val | V |
| Leucine | Leu | L | Isoleucine | Ile | I |
| Methionine | Met | M | Cysteine | Cys | C |
| Phenylalanine | Phe | F | Tyrosine | Tyr | Y |
| Tryptophan | Trp | W | Histidine | His | H |
| Lysine | Lys | K | Arginine | Arg | R |
| Glutamine | Glu | Q | Asparagine | Asn | N |
| Glutamic Acid | Glu | E | Aspartic Acid | Asp | D |
| Serine | Ser | S | Threonine | Thr | T |

### Overview

The present disclosure provides a SPINK9 polypeptide comprising a (e.g., at least one) mutant SPINK9 domain. The SPINK9 polypeptide inhibits, or otherwise attenuates, an active KLK through interaction of the mutant SPINK9 domain with the active KLK. Said active KLK may be any member of a family of KLKs comprising: KLK1, KLK2, KLK3, KLK4, KLK5, KLK6, KLK7, KLK8, KLK9, KLK10, KLK11, KLK12, KLK13, KLK14, KLK15, or any combination thereof. In some embodiments, the SPINK9 polypeptide inhibits the activity of any one of KLK5, KLK7, KLK14, or any combination thereof. In some embodiments, the SPINK9 polypeptide inhibits the activity of KLK5.

In some embodiments, a SPINK9 polypeptide is a selective inhibitor of an active KLK (e.g., a selective inhibitor of KLK5). The term “selective inhibitor” as used herein does not limit the inhibition to only a targeted KLK (e.g., KLK5) and a selective inhibitor may also inhibit the activity of additional KLK family members, or serine proteases, provided that the activity of the target KLK (e.g., KLK5) is inhibited to a greater degree (e.g., for example at least 20% or more such as 30%, 40%, 50%, 60%, 70%, 80%, 90%, or 95% or more) as compared to the activity of another KLK family member of other serine protease.

In a preferred embodiment, a SPINK9 polypeptide inhibits KLK5 and KLK14. In another preferred embodiment, a SPINK9 polypeptide is a selective inhibitor of KLK5 and KLK14.

In a preferred embodiment, a SPINK9 polypeptide inhibits KLK5. In another preferred embodiment, a SPINK9 polypeptide is a selective inhibitor of KLK5.

### Mutant SPINK9 Domains

A mutant SPINK9 domain may comprise all or a portion of the wild-type SPINK9 sequence of SEQ ID NO: 1 (which includes the signal peptide) or all or a portion of the wild-type SPINK9 sequence of SEQ ID NO: 2 (which excludes the signal sequence). In a preferred embodiment, the mutant SPINK9 domain comprise all or a portion of the wild-type SPINK9 of SEQ ID NO: 2.

(SEQ ID NO: 1)
MRATAIVLLLALTLATMFSIECAKQTKQMVDCSHYKKLPPGQQRFCHHM
YDPICGSDGKTYKNDCFFCSKVKKTDGTLKFVHFGKC
(SEQ ID NO: 2)
IECAKQTKQMVDCSHYKKLPPGQQRFCHHMYDPICGSDGKTYKNDCFFC
SKVKKTDGTLKFVHFGKC

In some embodiments, a mutant SPINK9 domain, or a biologically active fragment thereof, shares 50%, 55%, 60%, 65%, 70%, 75%, 80%, 85%, 90%, 91%, 92%, 93%, 94%, 95%, 96%, 97%, 98%, 99% or 100% sequence identity to SEQ ID NOS: 1 or 2. In certain embodiments, a mutant SPINK9 domain, or a biologically active fragment thereof, shares 70% or greater (i.e., 75%, 80%, 85%, 90%, or 95% or greater) sequence identity to SEQ ID NOS: 1 or 2. In a preferred embodiment, a mutant SPINK9 domain, or a biologically active fragment thereof, shares 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 2. In another preferred embodiment, a mutant SPINK9 domain, or a biologically active fragment thereof, 70% or greater (i.e., 75%, 80%, 85%, 90%, or 95% or greater) sequence identity to SEQ ID NO: 2.

A mutant SPINK9 domain may comprise one or more amino acid changes relative to SEQ ID NOS: 1 or 2. In certain embodiments, a mutant SPINK9 domain comprises from 1-20, 1-15, 1-10, 1-8, 1-7, 1-6, 1-5, 1-4, 1-3, 2, or 1 amino acid changes as compared to SEQ ID NOS: 1 or 2. In certain embodiments, a mutant SPINK9 domain comprises from 2-7, 3-7, 4-7, 5-7, 6-7, or 7 amino acid changes relative to SEQ ID NOS: 1 or 2. In certain embodiments, a mutant SPINK9 domain comprises from 2-15, (i.e., 2, 3, 4, 5, 6, 7, 8, 9, 10, 11, 12, 13, 14, or 15) amino acid changes relative to SEQ ID NOS: 1 or 2. Preferably a mutant SPINK9 domain comprises more than 1 and less than 20 (e.g., 2-7 or 2-15) amino acid changes relative to SEQ ID NOS: 1 or 2.

In some embodiments, at least one of the one or more amino acid changes in the mutant SPINK9 domain occur at or within amino acid positions 40 to 53 of SEQ ID NO: 1 or positions 21-34 of SEQ ID NO: 2. Preferably, at least one of the one or more amino acid changes in the mutant SPINK9 domain occur at or within amino acid positions 21-34 of SEQ ID NO: 2. The referenced amino acid positions contain the reactive P1/P1′ active site of SPINK9.

In some embodiments, at least one of the one or more amino acid changes in the mutant SPINK9 domain is a substitution or a deletion of one or more contiguous amino acids at or within positions 20-28 of SEQ ID NO: 1 and at least one of the one or more amino acid changes in the mutant SPINK9 domain is a substitution occurring at or within positions 40 to 53 of SEQ ID NO: 1. In some embodiments, at least one of the one or more amino acid changes in the mutant SPINK9 domain is a substitution or a deletion of one or more contiguous amino acids at or within positions 1-9 of SEQ ID NO: 2 and at least one of the one or more amino acid changes in the mutant SPINK9 domain is a substitution occurring at or within positions 21-34 of SEQ ID NO: 2. Preferably, at least one of the one or more amino acid changes in the mutant SPINK9 domain is a substitution or a deletion of one or more contiguous amino acids at positions 1-9 of SEQ ID NO: 2 and at least one of the one or more amino acid changes in the mutant SPINK9 domain is a substitution occurring at or within 21-34 of SEQ ID NO: 2.

In some embodiments, at least one of the one or more amino acid changes in the mutant SPINK9 domain is a substitution or a deletion of one or more contiguous amino acids at or within positions 20-24 of SEQ ID NO: 1 and at least one of the one or more amino acid changes in the mutant SPINK9 domain is a substitution occurring at or within positions 40 to 53 of SEQ ID NO: 1. In some embodiments, at least one of the one or more amino acid changes in the mutant SPINK9 domain is a substitution or a deletion of one or more contiguous amino acids at or within positions 1-5 of SEQ ID NO: 2 and at least one of the one or more amino acid changes in the mutant SPINK9 domain is a substitution occurring at or within positions 21-34 of SEQ ID NO: 2. Preferably, at least one of the one or more amino acid changes in the mutant SPINK9 domain is a substitution or a deletion of one or more contiguous amino acids at or within positions 1-5 of SEQ ID NO: 2 and at least one of the one or more amino acid changes in the mutant SPINK9 domain is a substitution occurring at or within 21-34 of SEQ ID NO: 2.

In some embodiments, at least 50% or more (e.g., 55%, 60%, 65%, 70%, 75%, 80%, 85%, 90%, or 95%) of the one or more amino acid changes in the mutant SPINK9 domain occur at or within amino acid positions 40 to 53 of SEQ ID NO: 1 or positions 21-34 of SEQ ID NO: 2. Preferably, at least 50% or more (e.g., 55%, 60%, 65%, 70%, 75%, 80%, 85%, 90%, or 95%) of the one or more amino acid changes in the mutant SPINK9 domain occur at or within amino acid positions 21-34 of SEQ ID NO: 2.

In one embodiment, the mutant SPINK9 domain comprises a sequence that shares 50%, 55%, 60%, 65%, 70%, 75%, 80%, 85%, 90%, 91%, 92%, 93%, 94%, 95%, 96%, 97%, 98%, 99% or 100% sequence identity to amino acid positions 1-20 and 35-67 of SEQ ID NO: 2 and contains at least one amino acid change (e.g., an amino acid substitution) at positions 21-34, of SEQ ID NO: 2. In a preferred embodiment, the sequence identity to amino acid positions 1-20 and 35-67 of SEQ ID NO: 2 is at least 70% (i.e., 70%, 75%, 80%, 85%, 90%, 91%, 92%, 93%, 94%, 95%, 96%, 97%, 98%, 99% or 100%).

In one embodiment, the mutant SPINK9 polypeptide comprises at least one mutant SPINK9 domain, wherein said at least one mutant SPINK9 domain comprises an amino acid sequence that shares at least 70% sequence identity with amino acids 1-20 and 35-67 set forth in SEQ ID NO: 2; wherein the mutant SPINK9 domain further comprises at least one amino acid substitution at positions 21-34 of SEQ ID NO: 2.

In one embodiment, the mutant SPINK9 polypeptide comprises, in an N-terminus to C-terminus order, a first amino acid sequence corresponding to amino acids 1-20 of SEQ ID NO: 2, a second amino acid sequence corresponding to amino acids 21-34 of SEQ ID NO: 2, and a third amino acid sequence corresponding to amino acids 35-67 of SEQ ID NO: 2.

In a first aspect of such an embodiment: (i) the first amino acid sequence shares at least 70% sequence identity to amino acids 1-20 of SEQ ID NO: 2; (ii) the second amino acid sequence comprises at least one amino acid substitution relative to amino acids 21-34 of SEQ ID NO: 2; and (iii) the third amino acid sequence shares at least 70% sequence identity to amino acids 35-67 of SEQ ID NO: 2 and optionally comprises an amino acid change (i.e., an amino acid substitution) at position 43.

In a second aspect of such embodiment: (i) the first amino acid sequence optionally contains the amino acids at positions 1, 2, 3, 4, 5, 6, 7, 8, or 9 of SEQ ID NO: 2; (ii) the second amino acid sequence comprises at least one amino acid change (e.g., an amino acid substitution) relative to positions 21-34 of SEQ ID NO: 2; and (iii) the third amino acid sequence shares at least 70% (i.e., 70%, 75%, 80%, 85%, 90%, 91%, 92%, 93%, 94%, 95%, 96%, 97%, 98%, 99% or 100%) sequence identity to SEQ ID NO: 2 and optionally comprises an amino acid change (i.e., an amino acid substitution) at position 43.

In a third aspect of such embodiment: (i) the first amino acid sequence optionally contains the amino acids at positions 1, 2, or 3 of SEQ ID NO: 2; (ii) the second amino acid sequence comprises at least one amino acid change (e.g., an amino acid substitution) at positions 21-34 of SEQ ID NO: 2; and (iii) the third amino acid sequence shares at least 70% (i.e., 70%, 75%, 80%, 85%, 90%, 91%, 92%, 93%, 94%, 95%, 96%, 97%, 98%, 99% or 100%) sequence identity to SEQ ID NO: 2 and optionally comprises an amino acid change (i.e., an amino acid substitution) at position 43.

In a fourth aspect of such embodiment: (i) the first amino acid sequence comprises a conservative amino acid substitution at any one of the positions 1, 2, 3, 4, 5, 6, 7, 8, or 9 of SEQ ID NO: 2 and optionally shares at least 70% sequence identity to amino acids 10-20 of SEQ ID NO: 2; (ii) the second amino acid sequence comprises at least one amino acid change (e.g., an amino acid substitution) at positions 21-34 of SEQ ID NO: 2; and (iii) the third amino acid sequence shares at least 70% (i.e., 70%, 75%, 80%, 85%, 90%, 91%, 92%, 93%, 94%, 95%, 96%, 97%, 98%, 99% or 100%) sequence identity to SEQ ID NO: 2 and optionally comprises an amino acid change (i.e., an amino acid substitution) at position 43.

In a fifth aspect of such embodiment: (i) the first amino acid sequence comprises a conservative amino acid substitution at any one of the positions 1, 2, or 3 of SEQ ID NO: 2 and optionally shares at least 70% sequence identity to amino acids 4-20 of SEQ ID NO: 2; (ii) the second amino acid sequence comprises at least one amino acid change (e.g., an amino acid substitution) at positions 21-34 of SEQ ID NO: 2; and (iii) the third amino acid sequence shares at least 70% (i.e., 70%, 75%, 80%, 85%, 90%, 91%, 92%, 93%, 94%, 95%, 96%, 97%, 98%, 99% or 100%) sequence identity to SEQ ID NO: 2 and optionally comprises an amino acid change (i.e., an amino acid substitution) at position 43.

In a sixth aspect of such embodiment: (i) the first amino acid sequence comprises a deletion of at least 1, 2, or 3 amino acids at positions 1, 2, or 3 of SEQ ID NO: 2 and optionally shares at least 70% sequence identity to amino acids 4-20 of SEQ ID NO: 2; (ii) the second amino acid sequence comprises at least one amino acid change (e.g., an amino acid substitution) at positions 21-34 of SEQ ID NO: 2; and (iii) the third amino acid sequence shares at least 70% (i.e., 70%, 75%, 80%, 85%, 90%, 91%, 92%, 93%, 94%, 95%, 96%, 97%, 98%, 99% or 100%) sequence identity to SEQ ID NO: 2 and optionally comprises an amino acid change (i.e., an amino acid substitution) at position 43.

In a seventh aspect of such embodiment: (i) the first amino acid sequence comprises a deletion of the amino acids at positions 1, 2, and 3 of SEQ ID NO: 2 and optionally shares at least 70% sequence identity to amino acids 4-20 of SEQ ID NO: 2; (ii) the second amino acid sequence comprises at least one amino acid change (e.g., an amino acid substitution) at positions 21-34 of SEQ ID NO: 2; and (iii) the sequence shares at least 70% (i.e., 70%, 75%, 80%, 85%, 90%, 91%, 92%, 93%, 94%, 95%, 96%, 97%, 98%, 99% or 100%) sequence identity to SEQ ID NO: 2 and optionally comprises an amino acid change (i.e., an amino acid substitution) at position 43.

In any one of the first to seventh aspects above, the second amino acid sequence comprises 1, 2, 3, 4, 5, 6, 7, 8, 9, or 10 amino acid changes (e.g., an amino acid substitution) at positions 21-34 of SEQ ID NO: 2.

In any one of the first to seventh aspects above, the second amino acid sequence comprises 1, 2, 3, 4, 5, 6, or 7 amino acid changes (e.g., an amino acid substitution) at positions 21-34 of SEQ ID NO: 2.

In any one of the first to seventh aspects above, the second amino acid sequence comprises 1, 2, 3, 4, 5, 6, or 7 amino acid changes (e.g., an amino acid substitution) at positions 22, 24, 25, 26, 28, 29, and 30 of SEQ ID NO: 2 and optionally one or more amino acid changes (e.g., an amino acid substitution) at positions 21, 23, 27, 31, 32, 33, and 34 of SEQ ID NO: 2.

In any one of the first to seventh aspects above, the second amino acid sequence comprises at least one amino acid change (e.g., 1, 2, 3, 4, 5, 6, or 7) at positions 22, 24, 25, 26, 28, 29, and 30 of SEQ ID NO: 2. For example, the second amino acid sequence may contain an amino acid change at only one position (e.g., position 28), at only 2 positions (e.g., positions 28 and 29), at only 3 positions (e.g., positions 28, 29 and 30), at only 4 positions (e.g., positions 25, 28, 29 and 30), at only 5 positions (e.g., positions 25, 26, 28, 29 and 30), at only 6 positions (e.g., positions 22, 25, 26, 28, 29 and 30), or at all 7 positions (e.g., positions 22, 24, 25, 26, 28, 29 and 30).

In any one of the first to seventh aspects above, the second amino acid sequence comprises 1, 2, 3, 4, 5, 6, or 7 amino acid changes (e.g., an amino acid substitution) at positions 22, 24, 25, 26, 28, 29, and 30 of SEQ ID NO: 2 and: (i) the amino acid at position 22 is selected from Gly, Glu, Met, Gln, Ala, Leu, Val, Asp, Asn, Ser, Lys, or His; (ii) the amino acid at position 24 is selected from Gln, Glu, Thr, Val, Leu, Ala, lie, Phe, Ser, or Tyr; (iii) the amino acid at position 25 is selected from Arg, Leu, Ile, His, Glu, Trp, Ser, Ala, Val, Tyr, Asp, or Met; (iv) the amino acid at position 26 is selected from Phe, Tyr, Trp, Met, His, Lys, or Asn; (v) the amino acid at position 28 is selected from His, Thr, Ser, or Gly; (vi) the amino acid at position 29 is selected from His, Arg, or Lys; (vii) the amino acid at position 30 is selected from Met, Glu, or Asp; or (viii) any combination thereof.

In any one of the first to seventh aspects above, the second amino acid sequence comprises 1, 2, 3, 4, 5, 6, or 7 amino acid substitutions at positions 22, 24, 25, 26, 28, 29, and 30 of SEQ ID NO: 2 and the second amino acid sequence comprises a substitution of: (i) Glu, Met, Gln, Ala, Leu, Val, Asp, Asn, Ser, Lys, or His amino acid for the amino acid relative to position 22 of SEQ ID NO: 2 or the amino acid at position 22 of SEQ ID NO: 2 is Gly; (ii) Glu, Thr, Val, Leu, Ala, Ile, Phe, Ser, or Tyr amino acid for the amino acid relative to position 24 of SEQ ID NO: 2 or the amino acid at position 24 of SEQ ID NO: 2 is Gin; (iii) Leu, Ile, His, Glu, Trp, Ser, Ala, Val, Tyr, Asp, or Met amino acid for the amino acid relative to position 25 of SEQ ID NO: 2 or the amino acid at position 25 of SEQ ID NO: 2 is Arg; (iv) Tyr, Trp, Met, His, Lys, or Asn amino acid for the amino acid relative to position 26 of SEQ ID NO: 2 or the amino acid at position 26 of SEQ ID NO: 2 is Phe; (v) Thr, Ser, or Gly amino acid for the amino acid relative to position 28 of SEQ ID NO: 2 or the amino acid at position 28 of SEQ ID NO: 2 is His; (vi) Arg or Lys amino acid for the amino acid relative to position 29 of SEQ ID NO: 2 or the amino acid at position 29 of SEQ ID NO: 2 is His; (vii) Glu or Asp amino acid for the amino acid relative to position 30 of SEQ ID NO: 2 or the amino acid at position 30 of SEQ ID NO: 2 is Met; or (viii) any combination thereof.

In any one of the first to seventh aspects above, the second amino acid sequence comprises 1, 2, 3, 4, 5, 6, or 7 amino acid changes (e.g., an amino acid substitution) at positions 22, 24, 25, 26, 28, 29, and 30 of SEQ ID NO: 2 and: (i) the amino acid at position 22 is selected from Gly, Glu, Met, Gln, Ala, Leu, Val, Asp, Asn, Ser, Lys, or His; (ii) the amino acid at position 24 is selected from Gln, Glu, Thr, Val, Leu, Ala, Ile, Phe, Ser, or Tyr; (iii) the amino acid at position 25 is selected from Arg, Leu, Ile, His, Glu, Trp, Ser, Ala, Val, Tyr, Asp, or Met; (iv) the amino acid at position 26 is selected from Phe, Tyr, Trp, Met, His, Lys, or Asn; (v) the amino acids at positions 28-30 are selected from Thr-Arg-Glu; Thr-Arg-Asp; Thr-Lys-Glu; Thr-Lys-Asp; Ser-Arg-Glu; or Gly-Arg-Asp; or (vi) any combination thereof.

In any one of the first to seventh aspects above, the second amino acid sequence comprises 1, 2, 3, 4, 5, 6, or 7 amino acid substitutions at positions 22, 24, 25, 26, 28, 29, and 30 of SEQ ID NO: 2 and the second amino acid sequence comprises a substitution of: (i) Glu, Met, Gln, Ala, Leu, Val, Asp, Asn, Ser, Lys, or His amino acid for the amino acid relative to position 22 of SEQ ID NO: 2 or the amino acid at position 22 of SEQ ID NO: 2 is Gly; (ii) Glu, Thr, Val, Leu, Ala, Ile, Phe, Ser, or Tyr amino acid for the amino acid relative to position 24 of SEQ ID NO: 2 or the amino acid at position 24 of SEQ ID NO: 2 is Gin; (iii) Leu, Ile, His, Glu, Trp, Ser, Ala, Val, Tyr, Asp, or Met amino acid for the amino acid relative to position 25 of SEQ ID NO: 2 or the amino acid at position 25 of SEQ ID NO: 2 is Arg; (iv) Tyr, Trp, Met, His, Lys, or Asn amino acid for the amino acid relative to position 26 of SEQ ID NO: 2 or the amino acid at position 26 of SEQ ID NO: 2 is Phe; (v) Thr-Arg-Glu; Thr-Arg-Asp; Thr-Lys-Glu; Thr-Lys-Asp; Ser-Arg-Glu; or Gly-Arg-Asp amino acids for the amino acids relative to positions 28-30 of SEQ ID NO: 2; or (vi) any combination thereof.

In any one of the first to seventh aspects above, the second amino acid sequence comprises 1, 2, 3, 4, 5, 6, or 7 amino acid changes (e.g., an amino acid substitution) at positions 22, 24, 25, 26, 28, 29, and 30 of SEQ ID NO: 2 and: (i) the amino acid at position 22 is selected from Gly, Glu, or Met; (ii) the amino acid at position 24 is selected from Gln, Glu, or Thr; (iii) the amino acid at position 25 is selected from Leu, Ile, His, Glu, or Trp; (iv) the amino acid at position 26 is selected from Phe, Tyr, or Trp; (v) the amino acid at position 28 is selected from Thr, Ser, or Gly; (vi) the amino acid at position 29 is selected from Arg, or Lys; (vii) the amino acid at position 30 is selected from Glu, or Asp; or (viii) any combination thereof.

In any one of the first to seventh aspects above, the second amino acid sequence comprises 1, 2, 3, 4, 5, 6, or 7 amino acid substitutions at positions 22, 24, 25, 26, 28, 29, and 30 of SEQ ID NO: 2 and the second amino acid sequence comprises a substitution of: (i) Glu or Met amino acid for the amino acid relative to position 22 of SEQ ID NO: 2 or the amino acid at position 22 of SEQ ID NO: 2 is Gly; (ii) Glu or Thr amino acid for the amino acid relative to position 24 of SEQ ID NO: 2 or the amino acid at position 24 of SEQ ID NO: 2 is Gln; (iii) Leu, lie, His, Glu or Trp amino acid for the amino acid relative to position 25 of SEQ ID NO: 2 or the amino acid at position 25 of SEQ ID NO: 2 is Arg; (iv) Tyr or Trp amino acid for the amino acid relative to position 26 of SEQ ID NO: 2 or the amino acid at position 26 of SEQ ID NO: 2 is Phe; (v) Thr, Ser, or Gly amino acid for the amino acid relative to position 28 of SEQ ID NO: 2 or the amino acid at position 28 of SEQ ID NO: 2 is His; (vi) Arg or Lys amino acid for the amino acid relative to position 29 of SEQ ID NO: 2 or the amino acid at position 29 of SEQ ID NO: 2 is His; (vii) Glu or Asp amino acid for the amino acid relative to position 30 of SEQ ID NO: 2 or the amino acid at position 30 of SEQ ID NO: 2 is Met; or (viii) any combination thereof.

In any one of the first to seventh aspects above, the second amino acid sequence comprises 1, 2, 3, 4, 5, 6, or 7 amino acid changes (e.g., an amino acid substitution) at positions 22, 24, 25, 26, 28, 29, and 30 of SEQ ID NO: 2 and: (i) the amino acid at position 22 is selected from Gly, Glu, or Met; (ii) the amino acid at position 24 is selected from Gln, Glu, or Thr; (iii) the amino acid at position 25 is selected from Leu, Ile, His, Glu, or Trp; (iv) the amino acid at position 26 is selected from Phe, Tyr, or Trp; (v) the amino acids at positions 28-30 are selected from Thr-Arg-Glu; Thr-Arg-Asp; Thr-Lys-Glu; Thr-Lys-Asp; Ser-Arg-Glu; or Gly-Arg-Asp; or (vi) any combination thereof.

In any one of the first to seventh aspects above, the second amino acid sequence comprises 1, 2, 3, 4, 5, 6, or 7 amino acid substitutions at positions 22, 24, 25, 26, 28, 29, and 30 of SEQ TD NO: 2 and the second amino acid sequence comprises a substitution of: (i) Glu or Met amino acid for the amino acid relative to position 22 of SEQ ID NO: 2 or the amino acid at position 22 of SEQ ID NO: 2 is Gly; (ii) Glu or Thr amino acid for the amino acid relative to position 24 of SEQ ID NO: 2 or the amino acid at position 24 of SEQ ID NO: 2 is Gin; (iii) Leu, Ile, His, Glu or Trp amino acid for the amino acid relative to position 25 of SEQ ID NO: 2 or the amino acid at position 25 of SEQ ID NO: 2 is Arg; (iv) Tyr or Trp amino acid for the amino acid relative to position 26 of SEQ ID NO: 2 or the amino acid at position 26 of SEQ ID NO: 2 is Phe; (v) Thr-Arg-Glu; Thr-Arg-Asp; Thr-Lys-Glu; Thr-Lys-Asp; Ser-Arg-Glu; or Gly-Arg-Asp amino acids for the amino acids relative to positions 28-30 of SEQ ID NO: 2; or (vi) any combination thereof.

In any one of the first to seventh aspects above, the optional amino acid change at position 43 of the third amino acid sequence is an amino acid substitution. In any one of the first to seventh aspects above, the third amino acid sequence comprises a substitution of a Gly amino acid for the amino acid relative to position 43 of SEQ ID NO: 2.

In any one of the first to seventh aspects above, at least one of the amino acid changes in SEQ ID NO: 2 is an amino acid substitution. In any one of the first to seventh aspects above, the amino acid change in SEQ ID NO: 2 is an amino acid substitution.

In some embodiments a mutant SPINK9 domain comprises, consists of, or consists essentially of an amino acid sequence represented by Formula I (SEQ ID NO: 3), provided that the amino acid sequence represented by Formula I does not correspond to a wild-type sequence of SPINK9 (i.e., SEQ ID NOS: 1 or 2).

(Formula I; SEQ ID NO: 3)
Ile Glu Cys Ala Lys Gln Thr Lys Gln Met Val Asp Cys Ser His Tyr Lys Lys Leu Pro
Pro X Gln X₂ X₃ X₄ Cys X₅ X₆ X₇ Tyr Asp Pro Ile Cys Gly Ser Asp Gly Lys Thr Tyr X₈
Asn Asp Cys PhePhe Cys Ser Lys Val Lys Lys Thr Asp Gly Thr Leu Lys Phe Val His Phe
Gly Lys Cys.

In some embodiments a mutant SPINK9 domain comprises, consists of, or consists essentially of an amino acid sequence represented by Formula III (SEQ ID NO: 135), provided that the amino acid sequence represented by Formula III does not correspond to a wild-type sequence of SPINK9 (i.e., SEQ ID NOS: 1 or 2).

(Formula I; SEQ ID NO: 135)
X₉ X₁₀ X₁₁ X₁₂ X₁₃ X₁₄ X₁₅ X₁₆ X₁₇ Met Val Asp Cys Ser His Tyr Lys Lys Leu Pro Pro X₁ Gln
X₂ X₃ X₄ Cys X₅ X₆ X₇ Tyr Asp Pro Ile Cys Gly Ser Asp Gly Lys Thr Tyr X₈ Asn Asp Cys
Phe Phe Cys Ser Lys Val Lys Lys Thr Asp Gly Thr Leu Lys Phe Val His Phe Gly Lys
Cys.

In some embodiments a mutant SPINK9 domain comprises, consists of, or consists essentially of an amino acid sequence represented by Formula IV (SEQ ID NO: 136), provided that the amino acid sequence represented by Formula IV does not correspond to a wild-type sequence of SPINK9 (i.e., SEQ ID NOS: 1 or 2).

(Formula I; SEQ ID NO: 136)
X₉ X₁₀ X₁₁ Ala Lys Gln Thr Lys Gln Met Val Asp Cys Ser His Tyr Lys Lys Leu Pro Pro
X₁ Gln X₂ X₃ X₄ Cys X₅ X₆ X₇ Tyr Asp Pro Ile Cys Gly Ser Asp Gly Lys Thr Tyr X₈ Asn
Asp Cys Phe Phe Cys Ser Lys Val Lys Lys Thr Asp Gly Thr Leu Lys Phe Val His Phe
Gly Lys Cys.

In a preferred embodiment, a mutant SPINK9 domain comprises, consists of, or consists essentially of an amino acid sequence represented by Formula I, Formula III, or Formula IV wherein X_{1-X17} are as indicated in Tables 1 or 2 herein.

TABLE 1
Representative Amino Acid Substitutions for
X₁-_{X17} of Formula I, Formula III, or Formula IV

| Position | Amino Acid Substitution |
|---|---|
| X₁ | Gly, Glu, Met, Gln, Ala, Leu, Val, Asp, Asn, Ser, Lys, or His |
| X₁ | Gly, Glu, or Met |
| X₂ | Gln, Glu, Thr, Val, Leu, Ala, Ile, Phe, Ser, or Tyr |
| X₂ | Gln, Glu, or Thr |
| X₃ | Arg, Leu, Ile, His, Glu, Trp, Ser, Ala, Val, Tyr, Asp, or Met |
| X₃ | Arg, Leu, Ile, His, Glu, or Trp |
| X₄ | Phe, Tyr, Trp, Met, His, Lys, or Asn |
| X₄ | Phe, Tyr, or Trp |
| X₅ | His, Thr, Ser, or Gly |
| X₅ | His or Thr |
| X₆ | His, Arg or Lys |
| X₆ | His or Arg |
| X₇ | Met, Glu or Asp |
| X₇ | Met or Glu |
| X₈ | Lys or Gly |
| X₉ | Absent or Ile |
| X₁₀ | Absent or Glu |
| X₁₁ | Absent or Cys |
| X₁₂ | Absent or Ala |
| X₁₃ | Absent or Lys |
| X₁₄ | Absent or Gln |
| X₁₅ | Absent or Thr |
| X₁₆ | Absent or Lys |
| X₁₇ | Absent or Gln |

TABLE 2
Representative Amino Acid Substitutions for X₁
-X₇ and X₁₉-X₂₇ of Formula I, Formula III, or Formula IV

| Position | Amino Acid Substitution |
|---|---|
| X₁ | Glu, Met, Gln, Ala, Leu, Val, Asp, Asn, Ser, Lys, or His |
| X₁ | Glu or Met |
| X₂ | Glu, Thr, Val, Leu, Ala, Ile, Phe, Ser, or Tyr |
| X₂ | Glu or Thr |
| X₃ | Leu, Ile, His, Glu, Trp, Ser, Ala, Val, Tyr, Asp, or Met |
| X₃ | Leu, Ile, His, Glu, or Trp |
| X₄ | Tyr, Trp, Met, His, Lys, or Asn |
| X₄ | Tyr or Trp |
| X₅ | Thr, Ser, or Gly |
| X₅ | Thr |
| X₆ | Arg or Lys |
| X₆ | Arg |
| X₇ | Glu or Asp |
| X₇ | Glu |
| X₈ | Lys |
| X₈ | Gly |
| X₉ | Absent |
| X₁₀ | Absent |
| X₁₁ | Absent |
| X₁₂ | Ala |
| X₁₃ | Lys |
| X₁₄ | Gln |
| X₁₅ | Thr |
| X₁₆ | Lys |
| X₁₇ | Gln |

In another embodiment a mutant SPINK9 domain comprises, consists of, or consists essentially of an amino acid sequence represented by Formula I, Formula III, or Formula IV wherein X₁-X₁₇ are as indicated in Tables 3 or 4 herein.

TABLE 3
Representative Amino Acid Substitutions for
X₁-_{X17} of Formula I, Formula III, or Formula IV

| Posi- | |
|---|---|
| tion | Amino Acid Substitution |
| X₁ | Gly, Glu, Met, Gln, Ala, Leu, Val, Asp, Asn, Ser, Lys, or His |
| X₁ | Gly, Glu, or Met |
| X₂ | Gln, Glu, Thr, Val, Leu, Ala, Ile, Phe, Ser, or Tyr |
| X₂ | Gln, Glu, or Thr |
| X₃ | Arg, Leu, Ile, His, Glu, Trp, Ser, Ala, Val, Tyr, Asp, or Met |
| X₃ | Arg, Leu, Ile, His, Glu, or Trp |
| X₄ | Phe, Tyr, Trp, Met, His, Lys, or Asn |
| X₄ | Phe, Tyr, or Trp |
| X₅- | Thr-Arg-Glu; Thr-Arg-Asp; Thr-Lys-Glu; Thr-Lys-Asp; Ser- |
| X₇ | Arg-Glu; or Gly-Arg-Asp. |
| X₈ | Lys or Gly |
| X₉ | Absent or Ile |
| X₁₀ | Absent or Glu |
| X₁₁ | Absent or Cys |
| X₁₂ | Absent or Ala |
| X₃₃ | Absent or Lys |
| X₄₄ | Absent or Gln |
| X₁₅ | Absent or Thr |
| X₁₆ | Absent or Lys |
| X₁₇ | Absent or Gln |

TABLE 4
Representative Amino Acid Substitutions for X₁-_{X17}
and X₁₉-X₂₇ of Formula I, Formula III, or Formula IV

| Posi- | |
|---|---|
| tion | Amino Acid Substitution |
| X₁ | Glu, Met, Gln, Ala, Leu, Val, Asp, Asn, Ser, Lys, or His |
| X₁ | Glu or Met |
| X₂ | Glu, Thr, Val, Leu, Ala, Ile, Phe, Ser, or Tyr |
| X₂ | Glu or Thr |
| X₃ | Leu, Ile, His, Glu, Trp, Ser, Ala, Val, Tyr, Asp, or Met |
| X₃ | Leu, Ile, His, Glu, or Trp |
| X₄ | Tyr, Trp, Met, His, Lys, or Asn |
| X₄ | Tyr or Trp |
| X₅- | Thr-Arg-Glu; Thr-Arg-Asp; Thr-Lys-Glu; Thr-Lys-Asp; Ser- |
| X₇ | Arg-Glu; or Gly-Arg-Asp. |
| X₈ | Lys |
| X₈ | Gly |
| X₉ | Absent |
| X₁₀ | Absent |
| X₁₁ | Absent |
| X₁₂ | Ala |
| X₁₃ | Lys |
| X₁₄ | Gln |
| X₁₅ | Thr |
| X₁₆ | Lys |
| X₁₇ | Gln |

In certain preferred embodiments, a mutant SPINK9 domain comprises, consists of, or consists essentially of an amino acid sequence selected from the group consisting of SEQ ID NOS: 4-23 and 115-134 as shown in Table 5.

TABLE 5
Exemplary Mutant SPINK9 domains

| | |
|---|---|
| Spink9-30 (SEQ ID | IECAKQTKQM VDCSHYKKLP PEQVSWCTRE YDPICGSDGK TYKNDCFFCS |
| NO: 4) | KVKKTDGTLK FVHFGKC |
| Spink9-112.K62G | IECAKQTKQM VDCSHYKKLP PEQELYCTRE YDPICGSDGK TYGNDCFFCS |
| (SEQ ID NO: 5) | KVKKTDGTLK FVHFGKC |
| Spink9-183 | IECAKOTKQM VDCSHYKKLP PQQQAYCTRD YDPICGSDGK TYKNDCFFCS |
| (SEQ ID NO: 6) | KVKKTDGTLK FVHFGKC |
| Spink9-186 | IECAKOTKQM VDCSHYKKLP PAQLIYCTKE YDPICGSDGK TYKNDCFFCS |
| (SEQ ID NO: 7) | KVKKTDGTLK FVHFGKC |
| Spink9-189 | IECAKQTKQM VDCSHYKKLP PGQEHFCTRE FDPICGSDGK TYKNDCFFCS |
| (SEQ ID NO: 8) | KVKKTDGTLK FVHFGKC |
| Spink9-236 | IECAKQTKQM VDCSHYKKLP PEQQLMCTRE YDPICGSDGK TYKNDCFFCS |
| (SEQ ID NO: 9) | KVKKTDGTLK FVHFGKC |
| Spink9-112 | IECAKQTKQM VDCSHYKKLP PEQELYCTRE YDPICGSDGK TYKNDCFFCS |
| (SEQ ID NO: 10) | KVKKTDGTLK FVHFGKC |
| Spink9-194 | IECAKQTKQM VDCSHYKKLP PMQTEWCTKE YDPICGSDGK TYKNDCFFCS |
| (SEQ ID NO: 11) | KVKKTDGTLK FVHFGKC |
| Spink9-245 | IECAKQTKQM VDCSHYKKLP PNQTVYCTRE YDPICGSDGK TYKNDCFFCS |
| (SEQ ID NO: 12) | KVKKTDGTLK FVHFGKC |
| Spink9-20 | IECAKQTKQM VDCSHYKKLP PLQEWYCTKD YDPICGSDGK TYKNDCFFCS |
| (SEQ ID NO: 13) | KVKKTDGTLK FVHFGKC |
| Spink9-21 | IECAKQTKQM VDCSHYKKLP PVQELYCTRE YDPICGSDGK TYKNDCFFCS |
| (SEQ ID NO: 14) | KVKKTDGTLK FVHFGKC |
| Spink9-73 | IECAKQTKQM VDCSHYKKLP PMQAYHCTRE YDPICGSDGK TYKNDCFFCS |
| (SEQ ID NO: 15) | KVKKTDGTLK FVHFGKC |
| Spink9-211 | IECAKOTKQM VDCSHYKKLP PGQEDFCTRE YDPICGSDGK TYKNDCFFCS |
| (SEQ ID NO: 16) | KVKKTDGTLK FVHFGKC |
| Spink9-212 | IECAKOTKQM VDCSHYKKLP PGQILYCGRD YDPICGSDGK TYKNDCFFCS |
| (SEQ ID NO: 17) | KVKKTDGTLK FVHFGKC |
| Spink9-237 | IECAKQTKQM VDCSHYKKLP PEQEWYCSRE YDPICGSDGK TYKNDCFFCS |
| (SEQ ID NO: 18) | KVKKTDGTLK FVHFGKC |
| Spink9-3 | IECAKQTKQM VDCSHYKKLP PDQFMKCTRE YDPICGSDGK TYKNDCFFCS |
| (SEQ ID NO: 19) | KVKKTDGTLK FVHFGKC |
| Spiak9-162 | IECAKQTKQM VDCSHYKKLP PNQTEFCSRE YDPICGSDGK TYKNDCFFCS |
| (SEQ ID NO: 20) | KVKKTDGTLK FVHFGKC |
| Spink9-193 | IECAKQTKQM VDCSHYKKLP PSQEHWCTRE YDPICGSDGK TYKNDCFFCS |
| (SEQ ID NO: 21) | KVKKTDGTLK FVHFGKC |
| Spink9-202 | IECAKQTKQM VDCSHYKKLP PKQSEYCTRE YDPICGSDGK TYKNDCFFCS |
| (SEQ ID NO: 22) | KVKKTDGTLK FVHFGKC |
| Spink9-74 | IECAKQTKQM VDCSHYKKLP PHQYINCTRE YDPICGSDGK TYKNDCFFCS |
| (SEQ ID NO: 23) | KVKKTDGTLK FVHFGKC |
| Spink9-30A | AKQTKQM VDCSHYKKLP PEQVSWCTRE YDPICGSDGK TYKNDCFFCS |
| (SEQ ID NO: 115) | KVKKTDGTLK FVHFGKC |
| Spink9-112.K62GA | AKQTKQM VDCSHYKKLP PEQELYCTRE YDPICGSDGK TYGNDCFFCS |
| (SEQ ID NO: 116) | KVKKTDGTLK FVHFGKC |
| Spink9-1834 | AKQTKQM VDCSHYKKLP PQQQAYCTRD YDPICGSDGK TYKNDCFFCS |
| (SEQ ID NO: 117) | KVKKTDGTLK FVHFGKC |
| Spink9-1864 | AKQTKQM VDCSHYKKLP PAQLIYCTKE YDPICGSDGK TYKNDCFFCS |
| (SEQ ID NO: 118) | KVKKTDGTLK FVHFGKC |
| Spink9-1894 | AKQTKQM VDCSHYKELP PGQEHFCTRE FDPICGSDGK TYKNDCFFCS |
| (SEQ ID NO: 119) | KVKKTDGTLK FVHFGKC |
| Spink9-2364 | AKQTKQM VDCSHYKKLP PEQQLMCTRE YDPICGSDGK TYKNDCFFCS |
| (SEQ ID NO: 120) | KVKKTDGTLK FVHFGKC |
| Spiak9-1124 | AKQTKQM VDCSHYKKLP PEQELYCTRE YDPICGSDGK TYKNDCFFCS |
| (SEQ ID NO: 121) | KVKKTDGTLK FVHFGKC |
| Spink9-1944 | AKQTKQM VDCSHYKKLP PMQTEWCTKE YDPICGSDGK TYKNDCFFCS |
| (SEQ ID NO: 122) | KVKKTDGTLK FVHFGKC |
| Spink9-245A | AKQTKQM VDCSHYKKLP PNQTVYCTRE YDPICGSDGK TYKNDCFFCS |
| (SEQ ID NO: 123) | KVKKTDGTLK FVHFGKC |
| Spink9-204 | AKQTKQM VDCSHYKKLP PLQEWYCTKD YDPICGSDGK TYKNDCFFCS |
| (SEQ ID NO: 124) | KVKKTDGTLK FVHFGKC |
| Spink9-21A | AKQTKQM VDCSHYKKLP PVQELYCTRE YDPICGSDGK TYKNDCFFCS |
| (SEQ ID NO: 125) | KVKKTDGTLK FVHFGKC |
| Spink9-734 | AKQTKQM VDCSHYKKLP PMQAYHCTRE YDPICGSDGK TYKNDCFFCS |
| (SEQ ID NO: 126) | KVKKTDGTLK FVHFGKC |
| Spink9-211A | AKQTKQM VDCSHYKKLP PGQEDFCTRE YDPICGSDGK TYKNDCFFCS |
| (SEQ ID NO: 127) | KVKKTDGTLK FVHFGKC |
| Spink9-2124 | AKQTKQM VDCSHYKKLP PGQILYCGRD YDPICGSDGK TYKNDCFFCS |
| (SEQ ID NO: 128) | KVKKTDGTLK FVHFGKC |
| Spink9-2374 | AKQTKQM VDCSHYKELP PEQEWYCSRE YDPICGSDGK TYKNDCFFCS |
| (SEQ ID NO: 129) | KVKKTDGTLK FVHFGKC |
| Spink9-34 | AKQTKQM VDCSHYKELP PDQFMKCTRE YDPICGSDGK TYKNDCFFCS |
| (SEQ ID NO: 130) | KVKKIDGTLK FVHFGKC |
| Spink9-1624 | AKQTKQM VDCSHYKKLP PNQTEFCSRE YDPICGSDGK TYKNDCFFCS |
| (SEQ ID NO: 131) | KVKKTDGTLK FVHFGKC |
| Spink9-1934 | AKQTKQM VDCSHYKKLP PSQEHWCTRE YDPICGSDGK TYKNDCFFCS |
| (SEQ ID NO: 132) | KVKKTDGTLK FVHFGKC |
| Spink9-2024 | AKQTKQM VDCSHYKKLP PKQSEYCTRE YDPICGSDGK TYKNDCFFCS |
| (SEQ ID NO: 133) | KVKKTDGTLK FVHFGKC |
| Spink9-744 | AKQTKQM VDCSHYKKLP PHQYINCTRE YDPICGSDGK TYKNDCFFCS |
| (SEQ ID NO: 134) | KVKKTDGTLK FVHFGKC |

In some embodiments, a mutant SPINK9 domain has a sequence disclosed herein (such as SEQ ID NOS; 4-23 and 115-134) further comprising 1 or more (e.g., from 1 to 20, 1 to 15, 1 to 10, 1 to 8, or 1 to 5) conservative sequence modifications, wherein the conservative sequence modification(s) is not at amino acid corresponding to amino acid positions 41, 43, 44, 45, 47, 48, or 49 of SEQ ID NO: 1 or positions 22, 24, 25, 26, 28, 29, or 30 of SEQ ID NO: 2. In some embodiments, a mutant SPINK9 domain has a sequence disclosed herein, further comprising 1 or more (e.g., from 1 to 20, 1 to 15, 1 to 10, 1 to 8, or 1 to 5) conservative sequence modifications, wherein the conservative sequence modification(s) is not at amino acid corresponding to amino acid positions 32, 41, 43, 44, 45, 46, 47, 48, 49, 54, 65, 68, or 86 of SEQ ID NO: 1 or positions 13, 22, 24, 25, 27, 26, 28, 29, 30, 35, 46, 49, or 67 of SEQ ID NO: 2. As used herein, the term “conservative sequence modifications” is intended to refer to amino acid modifications that do not significantly affect or alter the interaction of the mutant SPINK9 domain and a cognate peptide, e.g., a KLK protease, such as KLK5, and optionally includes natural variations of the amino acid sequence of SPINK9. Such conservative substitutions include, but are not limited to, amino acid substitutions, additions (e.g., additions of amino acids to the N or C terminus of the peptide) and deletions (e.g., deletions of amino acids from the N or C terminus of the peptide). In one embodiment, conservative amino acid modifications are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid and glutamic acid), neutral side chains (e.g., cysteine, serine, and threonine, asparagine, and glutamine), alkyl/aliphatic side chains (e.g., glycine, alanine, valine, methionine, leucine and isoleucine), and aromatic side chains (e.g., tyrosine, phenylalanine, and tryptophan). Thus, in certain embodiments, one or more amino acid residues of the peptides described herein can be replaced with other amino acid residues from the same side chain family and the altered peptide can be tested for retention of target binding/inhibition using methods known in the art. Modifications can be introduced by standard techniques known in the art, such as site-directed mutagenesis and PCR-mediated mutagenesis.

### SPINK9 Polypeptides

The present disclosure provides a SPINK9 polypeptide comprising a (e.g., at least one) mutant SPINK9 domain. In some embodiments, a SPINK9 polypeptide comprises a mutant SPINK9 domain and an (e.g., at least one) additional domain. Any SPINK9 polypeptide described herein may be provided as a pharmaceutically acceptable salt. Any mutant SPINK9 domain described herein may be used in a SPINK9 polypeptide.

The additional domains may be selected to provide or improve a property or characteristic of a SPINK9 polypeptide, including, but not limited to, to facilitate solubility, facilitate storage, increase in-vivo half-life, increase in-vivo stability, increase storage half-life, increase storage stability, reduce immunogenicity, reduce toxicity, provide for targeting to a specific cell type or types, provide for delayed or controlled release in-vivo, or any combination of the foregoing. Representative additional domains are discussed herein.

In some embodiments, a SPINK9 polypeptide comprises a single mutant SPINK9 domain, wherein the single mutant SPINK9 domain is any mutant SPINK9 domain disclosed herein, including, but not limited to, a mutant SPINK9 domain comprising an amino acid sequence of Formula I, III, or IV or an amino acid sequence set forth in Tables 1-5.

In some embodiments, a SPINK9 polypeptide comprises from two to twenty (e.g., 2, 3, 4, 5, 6, 7, 8, 9, 10, 11, 12, 13, 14, 15, 16, 17, 18, 19, or 20) independently selected mutant SPINK9 domains, wherein each mutant SPINK9 domain is any mutant SPINK9 domain disclosed herein, including, but not limited to, a mutant SPINK9 domain comprising an amino acid sequence of Formula I, III, or IV or an amino acid sequence set forth in Tables 1-5.

In some embodiments, a SPINK9 polypeptide comprises a first and a second independently selected mutant SPINK9 domain; and each of the first and the second mutant SPINK9 domains comprise an independently selected amino acid sequence selected from a mutant SPINK9 domain amino acid sequence as disclosed herein, including, but not limited to, an amino acid sequence of Formula I, III, or IV and an amino acid sequence set forth in Tables 1-5. In some such embodiments, the first and the second mutant SPINK9 domains are identical. In some such embodiments, the first and the second mutant SPINK9 domains are not identical. In a preferred embodiment, each mutant SPINK9 domain is independently selected from Table 5.

In some embodiments, a SPINK9 polypeptide comprises a first, a second, and a third independently selected mutant SPINK9 domain; and each of the first, the second, and the third mutant SPINK9 domains comprise an independently selected amino acid sequence selected from a mutant SPINK9 domain amino acid sequence as disclosed herein, including, but not limited to, an amino acid sequence of Formula I, III, or IV and an amino acid sequence set forth in Tables 1-5. In some such embodiments, two and only two of said mutant SPINK9 domains are identical. In some such embodiments, the first, the second, and the third mutant SPINK9 domains are identical. In some such embodiments, the first, the second, and the third mutant SPINK9 domains are not identical. In a preferred embodiment, each mutant SPINK9 domain is independently selected from Table 5.

When a SPINK9 polypeptide comprises more than one (e.g., from 2 to 20) mutant SPINK9 domains, the mutant SPINK9 domains may be positioned adjacent to one another in the SPINK9 polypeptide or an additional domain may be positioned between one or more mutant SPINK9 domains. In some embodiments, all mutant SPINK9 domains in a SPINK9 polypeptide are positioned adjacent to one another.

In some embodiments, when 2 or more adjacent mutant SPINK9 domains are present, the mutant SPINK9 domains are linked by direct fusion (e.g., there is no intervening amino acid sequence positioned between adjacent mutant SPINK9 domains). In some embodiments, when 2 or more adjacent mutant SPINK9 domains are present, the mutant SPINK9 domains are linked by a linker sequence. In some embodiments, when more than 2 mutant SPINK9 domains are present, the mutant SPINK9 domains are linked by a combination of direct fusion and a linker sequence. In some embodiments, at least one mutant SPINK9 domain of a SPINK9 polypeptide is linked to an adjacent mutant SPINK9 domain by a linker sequence. In other embodiments, at least one mutant SPINK9 domain of a SPINK9 polypeptide is linked to an adjacent mutant SPINK9 domain by direct fusion.

In some embodiments, a SPINK9 polypeptide comprises multiple associated polypeptide chains (e.g., a polypeptide dimer linked through a Fc portion or other dimerization domain), wherein at least one polypeptide chain in the SPINK9 polypeptide comprises at least one (e.g., from 2 to 20) mutant SPINK9 domain.

In some embodiments, a SPINK9 polypeptide comprises a first polypeptide chain and a second polypeptide chain linked through a Fc portion or other dimerization domain, wherein at least one of the first and second polypeptide chains comprises at least one (e.g., from 2 to 20) mutant SPINK9 domain. In some such embodiments, the mutant SPINK9 domains are independently selected from any mutant SPINK9 domain disclosed herein, including, but not limited to, a mutant SPINK9 domain comprising an amino acid sequence of Formula I, III, or IV or an amino acid sequence set forth in Tables 1-5. In a preferred embodiment, the first and second mutant SPINK9 domains are independently selected from Table 5.

In some embodiments, a SPINK9 polypeptide comprises a first polypeptide chain and a second polypeptide chain linked through a Fc portion or other dimerization domain, wherein the first polypeptide chain comprises a first mutant SPINK9 domain and the second polypeptide chain comprises a second mutant SPINK9 domain. In some such embodiments, the mutant SPINK9 domains are independently selected from any mutant SPINK9 domain disclosed herein, including, but not limited to, a mutant SPINK9 domain comprising an amino acid sequence of Formula I, III, or IV or an amino acid sequence set forth in Tables 1-5. In some such embodiments, the first and second mutant SPINK9 domains are identical. In some such embodiments, the first and second mutant SPINK9 domains are not identical. In a preferred embodiment, the first and second mutant SPINK9 domains are independently selected from Table 5.

In some embodiments, a SPINK9 polypeptide comprises a first polypeptide chain and a second polypeptide chain linked through a Fc portion or other dimerization domain, wherein the first polypeptide chain comprises a first and a second mutant SPINK9 domain and the second polypeptide chain comprises a third and a fourth mutant SPINK9 domain. In some such embodiments, the mutant SPINK9 domains are independently selected from any mutant SPINK9 domain disclosed herein, including, but not limited to, a mutant SPINK9 domain comprising an amino acid sequence of Formula I, III, or IV or an amino acid sequence set forth in Tables 1-5. In some such embodiments, at least two of the first to fourth mutant SPINK9 domains are identical. In some such embodiments, each of the first to fourth mutant SPINK9 domains are identical. In some such embodiments, each of the first to fourth mutant SPINK9 domains are not identical. In a preferred embodiment, the first to fourth mutant SPINK9 domains are independently selected from Table 5.

In some embodiments, a SPINK9 polypeptide comprises a first polypeptide chain and a second polypeptide chain linked through a Fc portion or other dimerization domain, wherein the first polypeptide chain comprises a first, second and third mutant SPINK9 domain and the second polypeptide chain comprises a fourth, fifth, and sixth mutant SPINK9 domain. In some such embodiments, the mutant SPINK9 domains are independently selected from any mutant SPINK9 domain disclosed herein, including, but not limited to, a mutant SPINK9 domain comprising an amino acid sequence of Formula I, III, or IV or an amino acid sequence set forth in Tables 1-5. In some such embodiments, at least two of the first to sixth mutant SPINK9 domains are identical. In some such embodiments, each of the first to sixth mutant SPINK9 domains are identical. In some such embodiments, each of the first to sixth mutant SPINK9 domains are not identical. In a preferred embodiment, the first to sixth mutant SPINK9 domains are independently selected from Table 5.

When a SPINK9 polypeptide comprises a first and a second polypeptide chain and the first and/or second polypeptide chains comprise more than one (e.g., from 2 to 20) mutant SPINK9 domains, the mutant SPINK9 domains in the first and/or second polypeptide chains may be positioned adjacent to one another in the first and/or second polypeptide chains or an additional domain may be positioned between one or more mutant SPINK9 domains in the first and second polypeptide chains. In some embodiments, all mutant SPINK9 domains in the first and/or second polypeptide chains are positioned adjacent to one another.

In some embodiments, when 2 or more adjacent mutant SPINK9 domains are present on the first and/or second polypeptide chains, the mutant SPINK9 domains are linked by direct fusion (e.g., there is no intervening amino acid sequence positioned between adjacent mutant SPINK9 domains). In some embodiments, when 2 or more adjacent mutant SPINK9 domains are present on the first and/or second polypeptide chains, the mutant SPINK9 domains are linked by a linker sequence. In some embodiments, when more than 2 mutant SPINK9 domains are present on the first and/or second polypeptide chains, the mutant SPINK9 domains are linked by a combination of direct fusion and a linker sequence. In some embodiments, at least one mutant SPINK9 domain of the first and/or second polypeptide chains is linked to an adjacent mutant SPINK9 domain by a linker sequence. In other embodiments, at least one mutant SPINK9 domain of first and/or second polypeptide chains is linked to an adjacent mutant SPINK9 domain by direct fusion.

A SPINK9 polypeptide may further comprise an additional domain. In some embodiments, a SPINK9 polypeptide comprises from 1 to 5 additional domains. Preferably, the additional domain(s) provide a SPINK9 polypeptide with an additional function in addition to inhibition of an active kallikrein-related peptidase (e.g., KLK5).

In a preferred embodiment, the additional domain is an immunoglobulin fragment crystallizable (Fc) region, or a functional fragment thereof. The Fc region has been shown to bind to the neonatal Fc receptor (FcRn) in endothelial cells that line the blood vessels, and, upon binding, the Fc region containing polypeptide is protected from degradation and re-released into the circulation, thereby increasing the half-life of a linked polypeptide after administration to a subject. An Fc region may be linked to a SPINK9 polypeptide to optimize the pharmacokinetic and pharmacodynamic properties of the polypeptide.

The term “Fc region” means the C-terminal region of an immunoglobulin heavy chain, which may be generated by papain digestion of an intact antibody. The Fc region may be a native amino acid sequence or may contain one or more modifications. The Fc region of an immunoglobulin generally comprises two constant domains, a CH2 domain and a CH3 domain, and optionally comprises a CH4 domain. Changes in the amino acid sequence of the Fc portion to alter antibody effector function are known in the art (e.g., U.S. Pat. Nos. 5,648,260 and 5,624,821). The Fc region mediates several important effector functions, including, but not limited to, cytokine induction, antibody dependent cell mediated cytotoxicity (ADCC), phagocytosis, complement dependent cytotoxicity (CDC), and increasing the half-life, and decreasing the clearance rate of a polypeptide. In some cases, these effector functions are desirable for a therapeutic immunoglobulin but in other cases might be unnecessary or even deleterious, depending on the therapeutic objectives.

In some embodiments, the Fc region, or functional fragment thereof, is linked to a SPINK9 polypeptide by direct fusion or by a linker sequence. In some embodiments, the Fc region, or functional fragment thereof, may be linked to a SPINK9 polypeptide at the N-terminus or the C-terminus of the mutant SPINK9 domain.

In some embodiments, the immunoglobulin Fc region is of human origin. In some embodiments, the immunoglobulin Fc region is from or derived from non-human animals, including, but not limited to, rodents such as, but not limited to, mice and rats; mammals, such as, but not limited to, rabbits, bovines, pigs, dogs, cynomolgus monkeys, marmosets, rhesus monkeys, and avians, such as, but not limited to, chickens. Preferably, the immunoglobulin Fc region is of human origin or is derived from non-human animals and is “humanized” as is known in the art. In some embodiments, the immunoglobulin Fc region comprises a human Fc sequence selected from the group consisting of an IgG1, IgG2, IgG3, IgG4, IgA, IgD, IgE, and IgM Fc region.

In some embodiments, a SPINK9 polypeptide comprises a modified Fc region. In some embodiments, a SPINK9 polypeptide comprises a modified Fc region of human origin. A modified Fc region may comprise amino acid insertions, deletions, substitutions, or chemical modifications. For example, an Fc region modification may be made to increase or decrease complement binding, to increase or decrease ADCC or CDC, to increase or decrease binding to FcR, to increase or decrease binding to FcRn, to modify glycosylation, or any combination thereof. Various Fc modifications are known in the art and have been described, for example, in Labrijin et al. (2009) Nature Biotech. 27(8):767-771; Greenwood et al. (1993) Eur. J. Immunol. 23:1098-1104; Mueller et al. (1997) Mol. Immunol. 34:441-452; Rother et al. (2007) Nature Biotechnol. 25: 1256-1264; Lee et al. (2019) Nature Comm. 10, article number 5031; and Saunders, Front. Immunol. (2019) article 1296). Any of the Fc modifications known in the art may be applied to a SPINK9 polypeptide comprising a Fc region disclosed herein. In some embodiments, an Fc modification is an amino acid substitution.

In some embodiments, a SPINK9 polypeptide comprising a modified Fc region is characterized by decreased binding (e.g., minimal binding or absence of binding) to a human Fc receptor (e.g., FcγRI, FcγRHA, FcγRHB, FcγRIIIB, or combinations thereof). In some embodiments, a SPINK9 polypeptide comprising a modified Fc region exhibits at least a 5% or greater (e.g., 10%, 15%, 20%, 30%, 40%, 50%, 60%, 70%, 80%, 90% or greater) reduction in Fc receptor binding as compared to a SPINK9 polypeptide comprising a wild-type Fc region. In some embodiments, a SPINK9 polypeptide comprising a modified Fc region exhibits at least a 5% or greater (e.g., 10%, 15%, 20%, 30%, 40%, 50%, 60%, 70%, 80%, 90% or greater) reduction in ADCC as compared to a SPINK9 polypeptide comprising a wild-type Fc region. In some embodiments, the reduction in ADCC is accomplished by the amino acid substitution L234A/L235A, G237A, N297A or any combination thereof (numbering according to the EU numbering; Edelman, G. M. et al., Proc. Natl. Acad. USA, 63, 78-85 (1969). PMID: 5257969).

In some embodiments, a SPINK9 polypeptide comprising a modified Fc region is characterized by decreased binding (e.g., minimal binding or absence of binding) to complement protein as compared to a SPINK9 polypeptide comprising a wild-type Fc reg. In some embodiments, a SPINK9 polypeptide comprising a modified Fc region exhibits at least a 5% or greater (e.g., 10%, 15%, 20%, 30%, 40%, 50%, 60%, 70%, 80%, 90% or greater) reduction in C1q binding as compared to a SPINK9 polypeptide comprising a wild-type Fc region. In some embodiments, a SPINK9 polypeptide comprising a modified Fc region exhibits at least a 5% or greater (e.g., 10%, 15%, 20%, 30%, 40%, 500%, 60%, 70%, 80%, 90% or greater) reduction CDC as compared to a SPINK9 polypeptide comprising a wild-type Fc region.

In some embodiments, a SPINK9 polypeptide comprising a modified Fc region is characterized by an increased affinity for human neonatal Fc receptors (FcRn) at low pH (e.g., pH 5.8-6.0) as compared to a SPINK9 polypeptide comprising a wild-type Fc region. In some embodiments, a SPINK9 polypeptide comprising a modified Fc region exhibits at least a 5% or greater (e.g., 10%, 15%, 20%, 30%, 40%, 50%, 60%, 70%, 80%, 90% or greater) affinity for FcRn at low pH as compared to a SPINK9 polypeptide comprising a wild-type Fc region. In some embodiments, the increased affinity for human neonatal Fc receptors (FcRn) at low pH is accomplished with the amino acid substitution M252Y/S254T/T256E, M428L/N434S, H433K/N434F, V264E/L309D/Q311H, V264E/L309D/Q311H/N434S, or L309D/Q311H/N434S (numbering according to the EU numbering; Edelman, G. M. et al., Proc. Natl. Acad. USA, 63, 78-85 (1969). PMID: 5257969).

In some embodiments, a SPINK9 polypeptide comprising a modified Fc region exhibits minimal or reduced glycosylation as compared to a SPINK9 polypeptide comprising a wild-type Fc region. In some embodiments, a SPINK9 polypeptide comprising a modified Fc region exhibits at least a 5% or greater (e.g., 10%, 15%, 20%, 30%, 40%, 50%, 60%, 70%, 80%, 90% or greater) reduction in glycosylation as compared to a SPINK9 polypeptide comprising a wild-type Fc region. In some embodiments, the minimal or reduced glycosylation is accomplished with the amino acid substitution N297X, where X is any amino acid other than N (e.g., a N297A substitution) (numbering according to the EU numbering (Edelman, G. M. et al., Proc. Natl. Acad. USA, 63, 78-85 (1969). PMID: 5257969).

In some embodiments, the Fc region is from an IgG1 antibody, particularly a human IgG1 antibody. In some embodiments, the modified Fc region is from an IgG1 antibody, particularly a human IgG1 antibody, and includes one or more of amino acid substitutions selected from the group consisting of: L234A/L235A, M252Y/S254T/T256E, M428L/N434S, H433K/N434F, V264E/L309D/Q311H, V264E/L309D/Q311H/N434S, L309D/Q311H/N434S, N297X, where X is any amino acid other than N (e.g., a N297A), P228S A330S, P331S, G237A, E233P/L234V/L235A, A327G/A330S/P331S, or L234F/L235E/P331S (numbering according to the EU numbering (Edelman, G. M. et al., Proc. Natl. Acad. USA, 63, 78-85 (1969). PMID: 5257969). In some embodiments, one or more additional mutations are included in such IgG1 modified Fc region. In some embodiments, a human IgG1 modified Fc region has up to 30 (e.g., 25, 20, 15, 10, 9, 8, 7, 6, 5 or 4) mutations as compared to wild-type human IgG1 Fc region sequence or fragment thereof.

In some embodiments, an Fc region comprises, consists of, or consists essentially of an amino acid sequence of Formula II (SEQ ID NO: 24):

Glu Pro Lys Ser Cys Asp Lys Thr His Thr Cys Pro X₁ Cys Pro Ala Pro X₂ X₃ X₄ Gly X₅ Pro Ser
Val Phe Leu Phe Pro Pro Lys Pro Lys Asp Thr Leu X₆ Ile X₇ Arg X₈ Pro Glu Val Thr Cys Val
Val X₉ Asp Val Ser His Glu Asp Pro Glu Val Lys Phe Asn Trp Tyr Val Asp Gly Val Glu Val
His Asn Ala Lys Thr Lys Pro Arg Glu Glu GIn Tyr X₁₀ Ser Thr Tyr Arg Val Val Ser Val Leu
Thr Val X₁₁ His X₁₂ Asp Trp Leu Asn Gly Lys Glu Tyr Lys Cys Lys Val Ser Asn Lys X₁₃ Leu
Pro X₁₄ X₁₅ Ile Glu Lys Thr Ile Ser Lys Ala Lys Gly Gln Pro Arg Glu Pro Gln Val Tyr Thr Leu
Pro Pro Ser Arg Glu Glu Met Thr Lys Asn Gln Val Ser Leu Thr Cys Leu Val Lys Gly Phe Tyr
Pro Ser Asp Ile Ala Val Glu Trp Glu Ser Asn Gly Gln Pro Glu Asn Asn Tyr Lys Thr Thr Pro
Pro Val Leu Asp Ser Asp Gly Ser Phe Phe Leu Tyr Ser Lys Leu Thr Val Asp Lys Ser Arg Trp
Gln Gln Gly Asn Val Phe Ser Cys Ser Val X₁₆ His Glu Ala Leu X₁₇ X₁₈ His Tyr Thr Gln Lys Ser
Leu Ser Leu Ser Pro Gly Lys;

wherein
X₁ is P or S;
X₂ is E or P;
X₃ and X₄ are each independently L or A;
X₅ is Q or A;
X₆ is M or Y;
X₇ is S or T;
X₈ is T or E;
X₉ is V or E;
X₁₀ is N or A
X₁₁ is L or D;
X₁₂ is Q or H;
X₁₃ is A or G;
X₁₄ is A or S;
X₁₅ is P or S;
X₁₆ is M or L;
X₁₇ is H or K; and
X₁₈ is N, S, or F; wherein wild-type amino acids are underlined, and non-underlined amino acids are non-wild type (substituted) amino acids.

In some embodiments of Formula II, X₆-X₇-X₈ is M-S-T, and at least one of X₁ to X₅ and X₉ to X₁₈ is a non-wild-type amino acid. In some embodiments of Formula II, X₆-X₇-X₈ is Y-T-E and at least one of X₁ to X₅ and X₉ to X₁₈ is a wild-type amino acid. In some embodiments of Formula II, X₆-X₇-X₈ is Y-T-E and each of X₁ to X₅ and X₉ to X₁₈ is a wild-type amino acid.

In some embodiments of Formula II, X₁₆ and X₁₈ are M and N, and at least one of X₁ to X₁₅ and X₁₇ is a non-wild-type amino acid. In some embodiments of Formula II, X₁₆ and X₁₈ are L and S, and at least one of X₁ to X₁₅ and X₁₇ is a wild-type amino acid. In some embodiments of Formula II, X₁₆ and X₁₈ are L and S, and each of X₁ to X₁₅ and X₁₇ is a wild-type amino acid.

In some embodiments of Formula II, X₃-X₄ is L-L, and at least one of X₁ to X₂ and X₅ to X₁₈ is a non-wild-type amino acid. In some embodiments of Formula II, X₃-X₄ is A-A, and at least one of X₁ to X₂ and X₅ to X₁₈ is a wild-type amino acid. In some embodiments of Formula II, X₃-X₄ is A-A, and each of X₁ to X₂ and X₅ to X₁₈ is a wild-type amino acid.

In some embodiments of Formula II, X₉, X₁₁, and X₁₂ are V, L, and Q, and at least one of X₁ to X₈, X₁₀, and X₁₃ to X₁₈ is a non-wild-type amino acid. In some embodiments of Formula II, X₉, X₁₁, and X₁₂ are E, D, and H, and at least one of X₁ to X₈, X₁₀, and X₁₃ to X₁₈ is a wild-type amino acid. In some embodiments of Formula II, X₉, X₁₁, and X₁₂ are E, D, and H, and each of X₁ to X₈, X₁₀, and X₁₃ to X₁₈ is a wild-type amino acid.

In some embodiments of Formula II, X₁₁, X₁₂ and X₁₈ are L, Q, and N, and at least one of X₁ to X₁₀ and X₁₃ to X₁₇ is a non-wild-type amino acid. In some embodiments of Formula II, X₁₁, X₁₂ and X₁₈ are D, H and S, and at least one of X₁ to X₁₀ and X₁₃ to X₁₇ is a wild-type amino acid. In some embodiments of Formula II, X₁₁, X₁₂ and X₁₈ are D, H and S, and each of X₁ to X₁₀ and X₁₃ to X₁₇ is a wild-type amino acid.

In some embodiments of Formula II, X₁₇-X₁₈ are H-N, and at least one of X₁ to X₁₆ is a non-wild-type amino acid. In some embodiments of Formula II, X₁₇-X₁₈ are K-F and at least one of X₁ to X₁₆ is a wild-type amino acid. In some embodiments of Formula II, X₁₇-X₁₈ are K-F and each of X₁ to X₁₆ is a wild-type amino acid.

In some embodiments of Formula II, X₁-X₄ is A-A, X₁₆ and X₁₈ are L and S, and each of X₁ to X₂, X₅ to X₁₅, and X₁₇ is a wild-type amino acid. In some embodiments of Formula II, X₃-X₄ is A-A, X₆-X₇-X₈ is Y-T-E, and each of X₁ to X₂, X₅, and X₉ to X₁₈ is a wild-type amino acid.

In some embodiments of Formula II, X₁₀ is N, and at least one of X₁ to X₉ and X₁₁ to X₁₈ is a non-wild-type amino acid. In some embodiments of Formula II, X₁₀ is A, and at least one of X₁ to X₉ and X₁₁ to X₁₈ is a wild-type amino acid. In some embodiments of Formula II, X₁₀ is A, and each of X₁ to X₉ and X₁₁ to X₁₈ is a wild-type amino acid.

In some embodiments of Formula II, X₁₀ is A, and X₃-X₄ is A-A, and at least one of or all of X₁ to X₂, X₅ to X₉, and X₁₁ to X₁₈ is a wild-type amino acid.

In some embodiments of Formula II, X₁₀ is A, and X₁₆ and X₁₈ are L and S, and at least one of or all of X₁ to X₉, X₁₁ to X₁₅, and X₁₇, is a wild-type amino acid.

In some embodiments of Formula II, X₁₀ is A, and X₆-X₇-X₈ is Y-T-E, and at least one of or all of X₁ to X₅, X₉, and X₁₁ to X₁₈, is a wild-type amino acid.

In some embodiments of Formula II, X₁₀ is A, X₃-X₄ is A-A, and X₁₆ and X₁₈ are L and S, and at least one of or all of X₁ to X₂, X₅ to X₉, X₁₁ to X₁₅, and X₁₇ is a wild-type amino acid.

In some embodiments of Formula II, X₁₀ is A, X₃-X₄ is A-A, and X₆-X₇-X₈ is Y-T-E, and at least one of or all of X₁ to X₂, X₅, and X₉ to X₁₈, is a wild-type amino acid.

As used in the present disclosure, a “linker” or “linker sequence” denotes an amino acid sequence of natural and/or synthetic origin. A linker sequence may comprise a linear amino acid chain wherein the 20 naturally occurring amino acids are the monomeric building blocks. The linker may have a length of from 1 to 50 amino acids (e.g., 1, 2, 3, 4, 5, 6, 7, 8, 9, 10, 10-15, 15-20, 20-30, 30-50 amino acids) and may comprise repetitive amino acid sequences or sequences of naturally occurring polypeptides. In some embodiments, the linker functions to ensure that polypeptides or polypeptide domains within a polypeptide linked to each other can perform their biological activity by allowing the polypeptides or polypeptide domains to fold correctly and/or to be presented properly. The linker may also function to provide a cleavage site between mutant SPINK9 domains and/or a mutant SPINK9 domain and an additional domain (e.g., an Fc region). In some embodiments, the linker sequence is rich in glycine, glutamine, and/or serine residues. These residues may be arranged in small repetitive units and may be repeated as needed. In some embodiments, a linker comprises a single amino acid, which is repeated between 10 to 20 times. In some embodiments, a linker comprises a cleavage site and the cleavage site is optionally positioned adjacent to a mutant SPINK9 domain (suitable cleavage sites are known in the art and described herein). For example, and without limitation, a SPINK9 polypeptide described herein may comprise any linker sequence known in the art, such as Gly-Gly-Ser repeat linkers, including GS, GGS, GGGS (SEQ ID NO: 25), GGGSGGGS (SEQ ID NO: 26), GGGSGGGSGGGS (SEQ ID NO: 27), GGGSGGGSGGGSGGGS (SEQ ID NO: 28), GGGSGGGSGGGSGGGSGGGS (SEQ ID NO: 29), GGGGS (SEQ ID NO: 30), GGGGSGGGGS (SEQ ID NO: 31), GGGGSGGGGSGGGGS (SEQ ID NO: 32), GGGGSGGGGSGGGGSGGGGS (SEQ ID NO: 33), and GGGGSGGGGSGGGGSGGGGSGGGGS (SEQ ID NO: 34).

In some embodiments, a linker sequence is encoded by a nucleic acid molecule and therefore can be recombinantly expressed. In some embodiments, a linker sequence includes one or more linkages that are not a peptide bond (such alternate linkages are known in the art and described herein). In such embodiments, a SPINK9 polypeptide containing such a linker sequence may be produced by non-recombinant methods as described herein or by a combination of recombinant and non-recombinant methods.

In some embodiments, the linker sequence comprises a cleavage site. Such cleavage sites may be recognized by a protease naturally present in a subject, such as a proprotein convertase (PPC), more preferably a subtilisin-like proprotein convertase (SPCs). SPCs are a family of calcium-dependent cleavage enzymes that act on dibasic sites of various peptide/protein substrates. An exemplary SPC is furin. Representative cleavage sites include, but are not limited to, KR ↓, RR ↓, RX(K/R)R ↓, RXXR ↓, KXXR ↓, RX (V/LXK/F/L) ↓, RNKR ↓ (SEQ ID NO: 35), KAKR ↓ (SEQ ID NO: 36), VFAQ ↓ SIP (SEQ ID NO: 37), and RXXRXX(R/K)R ↓ (SEQ ID NO: 38); where ↓ indicates point of cleavage. A cleavage site disclosed herein may be located at the N-terminus, C-terminus, or at an internal position of any linker sequence disclosed herein. In some embodiments, a cleavage site is located at the N-terminus or C-terminus of any linker sequence disclosed herein. In some embodiments, a cleavage site alone serves as a linker sequence.

In some embodiments, a SPINK9 polypeptide provided herein further comprises a signal sequence. In some such embodiments, the signal sequence is linked to the amino (N)-terminus of the polypeptide by direct fusion or by a linker sequence. In other embodiments, said signal sequence may be linked to the carboxy (C)-terminus of the polypeptide by direct fusion or by a linker sequence.

In some embodiments, the Fc region has an amino acid sequence comprising SEQ ID NO: 39 or a sequence sharing 50%, 55%, 60%, 65%, 70%, 75%, 80%, 85%, 90%, 91%, 92%, 93%, 94%, 95%, 96%, 97%, 98%, 99% or 100% sequence identity to SEQ ID NO: 39:

(SEQ ID NO: 39)
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK.

In some embodiments, the Fc region has an amino acid sequence comprising SEQ TD NO: 40 or a sequence sharing 50%, 55%, 60%, 65%, 70%, 75%, 80%, 85%, 90° %, 91%, 92%, 93%, 94%, 95%, 96%, 97%, 98%, 99% or 100% sequence identity to SEQ ID NO:40:

(SEQ ID NO: 40)
EPKSCDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK.

In some embodiments, the Fc region has an amino acid sequence comprising SEQ ID NO: 41 or a sequence sharing 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:41:

(SEQ ID NO: 41)
EPKSCDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVLHEALHSHYTQKSLSLSPGK.

In some embodiments, the Fc region has an amino acid sequence comprising SEQ ID NO: 42 or a sequence sharing 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:42:

(SEQ ID NO: 42)
EPKSCDKTHTCPPCPAPEAAGGPSVFLFPPKPKDTLYITREPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK.

In some embodiments, the Fc region has an amino acid sequence comprising SEQ ID NO: 43 or a sequence sharing 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:43:

(SEQ ID NO: 43)
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVLHEALHSHYTQKSLSLSPGK.

In some embodiments, the Fc region has an amino acid sequence comprising SEQ ID NO: 44 or a sequence sharing 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:44:

(SEQ ID NO: 44)
EPKSCDKTHTCPPCPAPELLGGPSVFLFPPKPKDTLYITREPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In some embodiments, the Fc region has an amino acid sequence comprising SEQ ID NO: 45 or a sequence sharing 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:45:

(SEQ ID NO: 45)
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALKFHYTQKSLSLSPGK.

In some embodiments, the Fc region has an amino acid sequence comprising SEQ ID NO: 46 or a sequence sharing 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:46:

(SEQ ID NO: 46)
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALKFHYTQKSLSLSPGK.

In some embodiments, the Fc region has an amino acid sequence comprising SEQ ID NO: 47 or a sequence sharing 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:47:

(SEQ ID NO: 47)
EPKSCDKTHTCPPCPAPELLGGPSVFLFPPKPKDTLMISRTPEVTCVVE
DVSHEDPEVKFNWYVDGVEVHNAKTKPREEQYNSTYRVVSVLTVDHHDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In some embodiments, the Fc region has an amino acid sequence comprising SEQ ID NO: 48 or a sequence sharing 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:48:

(SEQ ID NO: 48)
EPKSCDKTHTCPPCPAPEAAGGPSVFLFPPKPKDTLMISRTPEVTCVVE
DVSHEDPEVKFNWYVDGVEVHNAKTKPREEQYNSTYRVVSVLTVDHHDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In some embodiments, the Fc region has an amino acid sequence comprising SEQ ID NO: 49 or a sequence sharing 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:49:

(SEQ ID NO: 49)
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVDHHDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHSHYTQKSLSLSPGK.

In some embodiments, the Fc region has an amino acid sequence comprising SEQ ID NO: 50 or a sequence sharing 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:50:

(SEQ ID NO: 50)
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVDHHDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHSHYTQKSLSLSPGK.

In some embodiments, the Fc region has an amino acid sequence comprising SEQ TD NO: 51 or a sequence sharing 50%, 55%, 60%, 65%, 70%, 75%, 80%, 85%, 90° %, 91%, 92%, 93%, 94%, 95%, 96%, 97%, 98%, 99% or 100% sequence identity to SEQ ID NO:51:

(SEQ ID NO: 51)
EPKSCDKTHTCPPCPAPELLGGPSVFLFPPKPKDTLMISRTPEVTCVVV
DVSHEDPEVKFNWYVDGVEVHNAKTKPREEQYASTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In some embodiments, the Fc region has an amino acid sequence comprising SEQ ID NO: 52 or a sequence sharing 50%, 55%, 60%, 65%, 70%, 75%, 80%, 85%, 90%, 91%, 92%, 93%, 94%, 95%, 96%, 97%, 98%, 990% or 100% sequence identity to SEQ ID NO:52:

(SEQ ID NO: 52)
EPKSCDKTHTCPPCPAPEAAGGPSVFLFPPKPKDTLMISRTPEVTCVVV
DVSHEDPEVKFNWYVDGVEVHNAKTKPREEQYASTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In some embodiments, the Fc region has an amino acid sequence comprising SEQ ID NO: 53 or a sequence sharing 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:53:

(SEQ ID NO: 53)
EPKSCDKTHTCPPCPAPELLGGPSVFLFPPKPKDTLYITREPEVTCVVV
DVSHEDPEVKFNWYVDGVEVHNAKTKPREEQYASTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTOKSLSLSPGK.

In some embodiments, the Fc region has an amino acid sequence comprising SEQ ID NO: 54 or a sequence sharing 50% 55%, 60%, 65%, 70%, 75%, 80%, 85%, 90%, 91%, 92%, 93%, 94%, 95%, 96%, 97%, 98%, 99% or 100% sequence identity to SEQ ID NO:54:

(SEQ ID NO: 54)
EPKSCDKTHTCPPCPAPELLGGPSVFLFPPKPKDTLMISRTPEVTCVVV
DVSHEDPEVKFNWYVDGVEVHNAKTKPREEQYASTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVLHEALHSHYTQKSLSLSPGK.

In some preferred embodiments, a SPINK9 polypeptide comprises, consists of, or consists essentially of an amino acid sequence as described in Table 6.

TABLE 6

| Mutant | | |
|---|---|---|
| SPINK9 | Additional Mutant SPINK9 | Additional |
| domain | Domains | Domain |
| SEQ ID | 0-20, independently selected from | Any one of SEQ ID |
| NO: 4 | SEQ ID NOS: 4-23 or 115-134 | NOS: 24 and 39-54 |
| SEQ ID | 0-20, independently selected from | Any one of SEQ ID |
| NO: 5 | SEQ ID NOS: 4-23 or 115-134 | NOS: 24 and 39-54 |
| SEQ ID | 0-20, independently selected from | Any one of SEQ ID |
| NO: 6 | SEQ ID NOS: 4-23 or 115-134 | NOS: 24 and 39-54 |
| SEQ ID | 0-20, independently selected from | Any one of SEQ ID |
| NO: 7 | SEQ ID NOS: 4-23 or 115-134 | NOS: 24 and 39-54 |
| SEQ ID | 0-20, independently selected from | Any one of SEQ ID |
| NO: 8 | SEQ ID NOS: 4-23 or 115-134 | NOS: 24 and 39-54 |
| SEQ ID | 0-20, independently selected from | Any one of SEQ ID |
| NO: 9 | SEQ ID NOS: 4-23 or 115-134 | NOS: 24 and 39-54 |
| SEQ ID | 0-20, independently selected from | Any one of SEQ ID |
| NO: 10 | SEQ ID NOS: 4-23 or 115-134 | NOS: 24 and 39-54 |
| SEQ ID | 0-20, independently selected from | Any one of SEQ ID |
| NO: 11 | SEQ ID NOS: 4-23 or 115-134 | NOS: 24 and 39-54 |
| SEQ ID | 0-20, independently selected from | Any one of SEQ ID |
| NO: 12 | SEQ ID NOS: 4-23 or 115-134 | NOS: 24 and 39-54 |
| SEQ ID | 0-20, independently selected from | Any one of SEQ ID |
| NO: 13 | SEQ ID NOS: 4-23 or 115-134 | NOS: 24 and 39-54 |
| SEQ ID | 0-20, independently selected from | Any one of SEQ ID |
| NO: 14 | SEQ ID NOS: 4-23 or 115-134 | NOS: 24 and 39-54 |
| SEQ ID | 0-20, independently selected from | Any one of SEQ ID |
| NO: 15 | SEQ ID NOS: 4-23 or 115-134 | NOS: 24 and 39-54 |
| SEQ ID | 0-20, independently selected from | Any one of SEQ ID |
| NO: 16 | SEQ ID NOS: 4-23 or 115-134 | NOS: 24 and 39-54 |
| SEQ ID | 0-20, independently selected from | Any one of SEQ ID |
| NO: 17 | SEQ ID NOS: 4-23 or 115-134 | NOS: 24 and 39-54 |
| SEQ ID | 0-20, independently selected from | Any one of SEQ ID |
| NO: 18 | SEQ ID NOS: 4-23 or 115-134 | NOS: 24 and 39-54 |
| SEQ ID | 0-20, independently selected from | Any one of SEQ ID |
| NO: 19 | SEQ ID NOS: 4-23 or 115-134 | NOS: 24 and 39-54 |
| SEQ ID | 0-20, independently selected from | Any one of SEQ ID |
| NO: 20 | SEQ ID NOS: 4-23 or 115-134 | NOS: 24 and 39-54 |
| SEQ ID | 0-20, independently selected from | Any one of SEQ ID |
| NO: 21 | SEQ ID NOS: 4-23 or 115-134 | NOS: 24 and 39-54 |
| SEQ ID | 0-20, independently selected from | Any one of SEQ ID |
| NO: 22 | SEQ ID NOS: 4-23 or 115-134 | NOS: 24 and 39-54 |
| SEQ ID | 0-20, independently selected from | Any one of SEQ ID |
| NO: 23 | SEQ ID NOS: 4-23 or 115-134 | NOS: 24 and 39-54 |
| SEQ ID | 0-20, independently selected from | Any one of SEQ ID |
| NO: 115 | SEQ ID NOS: 4-23 or 115-134 | NOS: 24 and 39-54 |
| SEQ ID | 0-20, independently selected from | Any one of SEQ ID |
| NO: 116 | SEQ ID NOS: 4-23 or 115-134 | NOS: 24 and 39-54 |
| SEQ ID | 0-20, independently selected from | Any one of SEQ ID |
| NO: 117 | SEQ ID NOS: 4-23 or 115-134 | NOS: 24 and 39-54 |
| SEQ ID | 0-20, independently selected from | Any one of SEQ ID |
| NO: 118 | SEQ ID NOS: 4-23 or 115-134 | NOS: 24 and 39-54 |
| SEQ ID | 0-20, independently selected from | Any one of SEQ ID |
| NO: 119 | SEQ ID NOS: 4-23 or 115-134 | NOS: 24 and 39-54 |
| SEQ ID | 0-20, independently selected from | Any one of SEQ ID |
| NO: 120 | SEQ ID NOS: 4-23 or 115-134 | NOS: 24 and 39-54 |
| SEQ ID | 0-20, independently selected from | Any one of SEQ ID |
| NO: 121 | SEQ ID NOS: 4-23 or 115-134 | NOS: 24 and 39-54 |
| SEQ ID | 0-20, independently selected from | Any one of SEQ ID |
| NO: 122 | SEQ ID NOS: 4-23 or 115-134 | NOS: 24 and 39-54 |
| SEQ ID | 0-20, independently selected from | Any one of SEQ ID |
| NO: 123 | SEQ ID NOS: 4-23 or 115-134 | NOS: 24 and 39-54 |
| SEQ ID | 0-20, independently selected from | Any one of SEQ ID |
| NO: 124 | SEQ ID NOS: 4-23 or 115-134 | NOS: 24 and 39-54 |
| SEQ ID | 0-20, independently selected from | Any one of SEQ ID |
| NO: 125 | SEQ ID NOS: 4-23 or 115-134 | NOS: 24 and 39-54 |
| SEQ ID | 0-20, independently selected from | Any one of SEQ ID |
| NO: 126 | SEQ ID NOS: 4-23 or 115-134 | NOS: 24 and 39-54 |
| SEQ ID | 0-20, independently selected from | Any one of SEQ ID |
| NO: 127 | SEQ ID NOS: 4-23 or 115-134 | NOS: 24 and 39-54 |
| SEQ ID | 0-20, independently selected from | Any one of SEQ ID |
| NO: 128 | SEQ ID NOS: 4-23 or 115-134 | NOS: 24 and 39-54 |
| SEQ ID | 0-20, independently selected from | Any one of SEQ ID |
| NO: 129 | SEQ ID NOS: 4-23 or 115-134 | NOS: 24 and 39-54 |
| SEQ ID | 0-20, independently selected from | Any one of SEQ ID |
| NO: 130 | SEQ ID NOS: 4-23 or 115-134 | NOS: 24 and 39-54 |
| SEQ ID | 0-20, independently selected from | Any one of SEQ ID |
| NO: 131 | SEQ ID NOS: 4-23 or 115-134 | NOS: 24 and 39-54 |
| SEQ ID | 0-20, independently selected from | Any one of SEQ ID |
| NO: 132 | SEQ ID NOS: 4-23 or 115-134 | NOS: 24 and 39-54 |
| SEQ ID | 0-20, independently selected from | Any one of SEQ ID |
| NO: 133 | SEQ ID NOS: 4-23 or 115-134 | NOS: 24 and 39-54 |
| SEQ ID | 0-20, independently selected from | Any one of SEQ ID |
| NO: 134 | SEQ ID NOS: 4-23 or 115-134 | NOS: 24 and 39-54 |

In some embodiments, a SPINK9 polypeptide described by Table 6 has 0 additional mutant SPINK9 domains, wherein a linker sequence as described herein is optionally present to link one or more of the described domains. In certain embodiments, all the domains present are linked by direct fusion.

In some embodiments, a SPINK9 polypeptide described by Table 6 has from 1 to 20 (e.g., 1, 2, 3, 4, or 5. 5-10, 10-15, or 15-20) additional mutant SPINK9 domains, wherein a linker sequence as described herein is optionally present to link one or more of the described domains. In certain embodiments, all the domains present are linked by direct fusion.

In some embodiments, a SPINK9 polypeptide described by Table 6 has 2 additional mutant SPINK9 domains, wherein a linker sequence as described herein is optionally present to link one or more of the described domains. In certain embodiments, all the domains present are linked by direct fusion.

In some embodiments, at least one of a mutant SPINK9 domain is linked to an additional domain (e.g., an Fc region) of a SPINK9 polypeptide by a linker sequence. In some embodiments, at least one of a mutant SPINK9 domain is linked to an additional domain (e.g., an Fc region) of a SPINK9 polypeptide by direct fusion.

In some embodiments, at least one mutant SPINK9 domain of a SPINK9 polypeptide is linked to an adjacent mutant SPINK9 domain by a linker sequence and a mutant SPINK9 domain is linked to an additional domain (e.g., an Fc region) by a linker sequence or by direct fusion. In some embodiments, at least one mutant SPINK9 domain of a SPINK9 polypeptide is linked to an adjacent mutant SPINK9 domain by direct fusion and a mutant SPINK9 domain is linked to an additional domain (e.g., an Fc region) by a linker sequence or by direct fusion.

In some preferred embodiments, a SPINK9 polypeptide comprises, consists of, or consists essentially of an amino acid sequence selected from the group consisting of SEQ ID NOS: 55-114 as shown in Table 7 below.

TABLE 7
Exemplary mutant SPINK9 domain-containing Polypeptides

| | |
|---|---|
| Spink9-30.Fc | IECAKQTKQMVDCSHYKKLPPEQVSWCTREYDPICGSDGKTYKNDCFFCSKVKKTDG |
| (SEQ ID NO: | TLKFVHFGKCEPKSCDKTHTCPPCPAPEAAGGPSVFLFPPKPKDTLMISRTPEVTCVVV |
| 55) | DVSHEDPEVKFNWYVDGVEVHNAKTKPREEQYNSTYRVVSVLTVLHQDWLNGKEYK |
| | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPEQELYCTREYDPICGSDGKTYGNDCFFCSKVKKTDGT |
| 112.K62G.Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 56) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPQQQAYCTRDYDPICGSDGKTYKNDCFFCSKVKKTDG |
| 183.Fc | TLKFVHFGKCEPKSCDKTHTCPPCPAPEAAGGPSVFLFPPKPKDTLMISRTPEVTCVVV |
| (SEQ ID NO: | DVSHEDPEVKFNWYVDGVEVHNAKTKPREEQYNSTYRVVSVLTVLHQDWLNGKEYK |
| 57) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPAQLIYCTKEYDPICGSDGKTYKNDCFFCSKVKKTDGT |
| 186.Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 58) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPGQEHFCTREFDPICGSDGKTYKNDCFFCSKVKKTDGT |
| 189.Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 59) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPEQQLMCTREYDPICGSDGKTYKNDCFFCSKVKKTDG |
| 236.Fc | TLKFVHFGKCEPKSCDKTHTCPPCPAPEAAGGPSVFLFPPKPKDTLMISRTPEVTCVVV |
| (SEQ ID NO: | DVSHEDPEVKFNWYVDGVEVHNAKTKPREEQYNSTYRVVSVLTVLHQDWLNGKEYK |
| 60) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPEQELYCTREYDPICGSDGKTYKNDCFFCSKVEKTDGT |
| 112.Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 61) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPMQTEWCTKEYDPICGSDGKTYKNDCFFCSKVKKTDG |
| 194.Fc | TLKFVHFGKCEPKSCDKTHTCPPCPAPEAAGGPSVFLFPPKPKDTLMISRTPEVTCVVV |
| (SEQ ID NO: | DVSHEDPEVKFNWYVDGVEVHNAKTKPREEQYNSTYRVVSVLTVLHQDWLNGKEYK |
| 62) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPNQTVYCTREYDPICGSDGKTYKNDCFFCSKVKKTDG |
| 245.Fc | TLKFVHFGKCEPKSCDKTHTCPPCPAPEAAGGPSVFLFPPKPKDTLMISRTPEVTCVVV |
| (SEQ ID NO: | DVSHEDPEVKFNWYVDGVEVHNAKTKPREEQYNSTYRVVSVLTVLHQDWLNGKEYK |
| 63) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9-20.Fc | IECAKQTKQMVDCSHYKKLPPLQEWYCTKDYDPICGSDGKTYKNDCFFCSKVKKTDG |
| (SEQ ID NO: | TLKFVHFGKCEPKSCDKTHTCPPCPAPEAAGGPSVFLFPPKPKDTLMISRTPEVTCVVV |
| 64) | DVSHEDPEVKFNWYVDGVEVHNAKTKPREEQYNSTYRVVSVLTVLHQDWLNGKEYK |
| | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9-21.Fc | IECAKQTKQMVDCSHYKKLPPVQELYCTREYDPICGSDGKTYKNDCFFCSKVKKTDGT |
| (SEQ ID NO: | LKFVHFGKCEPKSCDKTHTCPPCPAPEAAGGPSVFLFPPKPKDTLMISRTPEVTCVVVD |
| 65) | VSHEDPEVKFNWYVDGVEVHNAKTKPREEQYNSTYRVVSVLTVLHQDWLNGKEYKC |
| | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9-73.Fc | IECAKQTKQMVDCSHYKKLPPMQAYHCTREYDPICGSDGKTYKNDCFFCSKVKKTDG |
| (SEQ ID NO: | TLKFVHFGKCEPKSCDKTHTCPPCPAPEAAGGPSVFLFPPKPKDTLMISRTPEVTCVVV |
| 66) | DVSHEDPEVKFNWYVDGVEVHNAKTKPREEQYNSTYRVVSVLTVLHQDWLNGKEYK |
| | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPGQEDFCTREYDPICGSDGKTYKNDCFFCSKVKKTDGT |
| 211.Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 67) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPGQILYCGRDYDPICGSDGKTYKNDCFFCSKVKKTDGT |
| 212.Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 68) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPEQEWYCSREYDPICGSDGKTYKNDCFFCSKVKKTDG |
| 237.Fc | TLKFVHFGKCEPKSCDKTHTCPPCPAPEAAGGPSVFLFPPKPKDTLMISRTPEVTCVVV |
| (SEQ ID NO: | DVSHEDPEVKFNWYVDGVEVHNAKTKPREEQYNSTYRVVSVLTVLHQDWLNGKEYK |
| 69) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9-3.Fc | IECAKQTKQMVDCSHYKKLPPDQFMKCTREYDPICGSDGKTYKNDCFFCSKVKKTDG |
| (SEQ ID NO: | TLKFVHFGKCEPKSCDKTHTCPPCPAPEAAGGPSVFLFPPKPKDTLMISRTPEVTCVVV |
| 70) | DVSHEDPEVKFNWYVDGVEVHNAKTKPREEQYNSTYRVVSVLTVLHQDWLNGKEYK |
| | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPNQTEFCSREYDPICGSDGKTYKNDCFFCSKVKKTDGT |
| 162.Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 71) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPSQEHWCTREYDPICGSDGKTYKNDCFFCSKVKKTDG |
| 193.Fc | TLKFVHFGKCEPKSCDKTHTCPPCPAPEAAGGPSVFLFPPKPKDTLMISRTPEVTCVVV |
| (SEQ ID NO: | DVSHEDPEVKFNWYVDGVEVHNAKTKPREEQYNSTYRVVSVLTVLHQDWLNGKEYK |
| 72) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPKQSEYCTREYDPICGSDGKTYKNDCFFCSKVKKTDGT |
| 202.Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 73) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9-74.Fc | IECAKQTKQMVDCSHYKKLPPHQYINCTREYDPICGSDGKTYKNDCFFCSKVKKTDGT |
| (SEQ ID NO: | LKFVHFGKCEPKSCDKTHTCPPCPAPEAAGGPSVFLFPPKPKDTLMISRTPEVTCVVVD |
| 74) | VSHEDPEVKFNWYVDGVEVHNAKTKPREEQYNSTYRVVSVLTVLHQDWLNGKEYKC |
| | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVLHEALHSHYT |
| | QKSLSLSPGK |
| Spink9- | IECAKQTKQMVDCSHYKKLPPEQVSWCTREYDPICGSDGKTYKNDCFFCSKVKKTDG |
| 30.2Fc | TLKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 75) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPEQELYCTREYDPICGSDGKTYGNDCFFCSKVKKTDGT |
| 112.2K62G.Fc | LKFVHFGKCEPKSCDKTHTCPPCPAPEAAGGPSVFLFPPKPKDTLYTTREPEVTCVVVD |
| (SEQ ID NO: | VSHEDPEVKFNWYVDGVEVHNAKTKPREEQYNSTYRVVSVLTVLHQDWLNGKEYKC |
| 76) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPQQQAYCTRDYDPICGSDGKTYKNDCFFCSKVKKTDG |
| 183.2Fc | TLKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 77) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPAQLIYCTKEYDPICGSDGKTYKNDCFFCSKVKKTDGT |
| 186.2Fc | LKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 78) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPGQEHFCTREFDPICGSDGKTYKNDCFFCSKVKKTDGT |
| 189.2Fc | LKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 79) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPEQQLMCTREYDPICGSDGKTYKNDCFFCSKVKKTDG |
| 236,2Fc | TLKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 80) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPEQELYCTREYDPICGSDGKTYKNDCFFCSKVKKTDGT |
| 112.2Fc | LKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 81) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPMQTEWCTKEYDPICGSDGKTYKNDCFFCSKVKKTDG |
| 194.2Fc | TLKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 82) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPNQTVYCTREYDPICGSDGKTYKNDCFFCSKVKKTDG |
| 245.2Fc | TLKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 83) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPLQEWYCTKDYDPICGSDGKTYKNDCFFCSKVKKTDG |
| 20.2Fc | TLKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 84) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPVQELYCTREYDPICGSDGKTYKNDCFFCSKVKKTDGT |
| 21.2Fc | LKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 85) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPMQAYHCTREYDPICGSDGKTYKNDCFFCSKVKKTDG |
| 73.2Fc | TLKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 86) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPGQEDFCTREYDPICGSDGKTYKNDCFFCSKVKKTDGT |
| 211.2Fc | LKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 87) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPGQILYCGRDYDPICGSDGKTYKNDCFFCSKVKKTDGT |
| 212.2Fc | LKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 88) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPEQEWYCSREYDPICGSDGKTYKNDCFFCSKVKKTDG |
| 237.2Fc | TLKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 89) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9-3.2Fc | IECAKQTKQMVDCSHYKKLPPDQFMKCTREYDPICGSDGKTYKNDCFFCSKVKKTDG |
| (SEQ ID NO: | TLKFVHFGKCEPKSCDKTHTCPPCPAPEAAGGPSVFLFPPKPKDTLYITREPEVTCVVVD |
| 90) | VSHEDPEVKFNWYVDGVEVHNAKTKPREEQYNSTYRVVSVLTVLHQDWLNGKEYKC |
| | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPNQTEFCSREYDPICGSDGKTYKNDCFFCSKVKKTDGT |
| 162.2Fc | LKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 91) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPSQEHWCTREYDPICGSDGKTYKNDCFFCSKVKKTDG |
| 193.2Fc | TLKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 92) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPKQSEYCTREYDPICGSDGKTYKNDCFFCSKVKKIDGT |
| 202.2Fc | LKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 93) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPHQYINCTREYDPICGSDGKTYKNDCFFCSKVKKTDGT |
| 74.2Fc | LKFVHFGKCEPKSCDKTHTCPPCPAPEAAGGPSVFLFPPKPKDTLYITREPEVTCVVVD |
| (SEQ ID NO: | VSHEDPEVKFNWYVDGVEVHNAKTKPREEQYNSTYRVVSVLTVLHQDWLNGKEYKC |
| 94) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPEQVSWCTREYDPICGSDGKTYKNDCFFCSKVKKTDG |
| 30.3Fc | TLKFVHFGKCEPKSCDKTHTCPPCPAPEAAGGPSVFLFPPKPKDTLMISRTPEVTCVVV |
| (SEQ ID NO: | DVSHEDPEVKFNWYVDGVEVHNAKTKPREEQYNSTYRVVSVLTVLHQDWLNGKEYK |
| 95) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPEQELYCTREYDPICGSDGKTYGNDCFFCSKVKKTDGT |
| 112.3K62G.Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 96) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPQQQAYCTRDYDPICGSDGKTYKNDCFFCSKVKKTDG |
| 183.3Fc | TLKFVHFGKCEPKSCDKTHTCPPCPAPEAAGGPSVFLFPPKPKDTLMISRTPEVTCVVV |
| (SEQ ID NO: | DVSHEDPEVKFNWYVDGVEVHNAKTKPREEQYNSTYRVVSVLTVLHQDWLNGKEYK |
| 97) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPAQLIYCTKEYDPICGSDGKTYKNDCFFCSKVKKTDGT |
| 186.3Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 98) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPGQEHFCTREFDPICGSDGKTYKNDCFFCSKVKKTDGT |
| 189.3Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 99) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPEQQLMCTREYDPICGSDGKTYKNDCFFCSKVKKTDG |
| 236.3Fc | TLKFVHFGKCEPKSCDKTHTCPPCPAPEAAGGPSVFLFPPKPKDTLMISRTPEVTCVVV |
| (SEQ ID NO: | DVSHEDPEVKFNWYVDGVEVHNAKTKPREEQYNSTYRVVSVLTVLHQDWLNGKEYK |
| 100) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPEQELYCTREYDPICGSDGKTYKNDCFFCSKVEKTDGT |
| 112.3Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 101) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9. | IECAKQTKQMVDCSHYKKLPPMQTEWCTKEYDPICGSDGKTYKNDCFFCSKVKKTDG |
| 194.3Fe | TLKFVHFGKCEPKSCDKTHTCPPCPAPEAAGGPSVFLFPPKPKDTLMISRTPEVTCVVV |
| (SEQ ID NO: | DVSHEDPEVKFNWYVDGVEVHNAKTKPREEQYNSTYRVVSVLTVLHQDWLNGKEYK |
| 102) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPNQTVYCTREYDPICGSDGKTYKNDCFFCSKVKKTDG |
| 245.3Fc | TLKFVHFGKCEPKSCDKTHTCPPCPAPEAAGGPSVFLFPPKPKDTLMISRTPEVTCVVV |
| (SEQ ID NO: | DVSHEDPEVKFNWYVDGVEVHNAKTKPREEQYNSTYRVVSVLTVLHQDWLNGKEYK |
| 103) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPLQEWYCTKDYDPICGSDGKTYKNDCFFCSKVKKTDG |
| 20.3Fc | TLKFVHFGKCEPKSCDKTHTCPPCPAPEAAGGPSVFLFPPKPKDTLMISRTPEVTCVVV |
| (SEQ ID NO: | DVSHEDPEVKFNWYVDGVEVHNAKTKPREEQYNSTYRVVSVLTVLHQDWLNGKEYK |
| 104) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPVQELYCTREYDPICGSDGKTYKNDCFFCSKVKKTDGT |
| 21.3Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 105) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPMQAYHCTREYDPICGSDGKTYKNDCFFCSKVKKTDG |
| 73.3Fc | TLKFVHFGKCEPKSCDKTHTCPPCPAPEAAGGPSVFLFPPKPKDTLMISRTPEVTCVVV |
| (SEQ ID NO: | DVSHEDPEVKFNWYVDGVEVHNAKTKPREEQYNSTYRVVSVLTVLHQDWLNGKEYK |
| 106) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPGQEDFCTREYDPICGSDGKTYKNDCFFCSKVKKTDGT |
| 211.3Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 107) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPGQILYCGRDYDPICGSDGKTYKNDCFFCSKVKKTDGT |
| 212.3Fc | LKFVHFGKCEPKSCDKTHTCPPCPAPEAAGGPSVFLFPPKPKDTLMISRTPEVTCVVVD |
| (SEQ ID | VSHEDPEVKFNWYVDGVEVHNAKTKPREEQYNSTYRVVSVLTVLHQDWLNGKEYKC |
| NO: 108) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPEQEWYCSREYDPICGSDGKTYKNDCFFCSKVKKTDG |
| 237.3Fc | TLKFVHFGKCEPKSCDKTHTCPPCPAPEAAGGPSVFLFPPKPKDTLMISRTPEVTCVVV |
| (SEQ ID NO: | DVSHEDPEVKFNWYVDGVEVHNAKTKPREEQYNSTYRVVSVLTVLHQDWLNGKEYK |
| 109) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9-3.3Fc | IECAKQTKQMVDCSHYKKLPPDQFMKCTREYDPICGSDGKTYKNDCFFCSKVKKTDG |
| (SEQ ID NO: | TLKFVHFGKCEPKSCDKTHTCPPCPAPEAAGGPSVFLFPPKPKDTLMISRTPEVTCVVV |
| 110) | DVSHEDPEVKFNWYVDGVEVHNAKTKPREEQYNSTYRVVSVLTVLHQDWLNGKEYK |
| | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPNQTEFCSREYDPICGSDGKTYKNDCFFCSKVKKTDGT |
| 162.3Fe | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 111) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPSQEHWCTREYDPICGSDGKTYKNDCFFCSKVKKTDG |
| 193.3Fc | TLKFVHFGKCEPKSCDKTHTCPPCPAPEAAGGPSVFLFPPKPKDTLMISRTPEVTCVVV |
| (SEQ ID | DVSHEDPEVKFNWYVDGVEVHNAKTKPREEQYNSTYRVVSVLTVLHQDWLNGKEYK |
| NO: 112) | CKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPKQSEYCTREYDPICGSDGKTYKNDCFFCSKVKKTDGT |
| 202.3Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 113) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |
| Spink9- | IECAKQTKQMVDCSHYKKLPPHQYINCTREYDPICGSDGKTYKNDCFFCSKVKKTDGT |
| 74.3Fc | LKFVHFGKCEPKSCDKTHTCPPCPAPEAAGGPSVFLFPPKPKDTLMISRTPEVTCVVVD |
| (SEQ ID NO: | VSHEDPEVKFNWYVDGVEVHNAKTKPREEQYNSTYRVVSVLTVLHQDWLNGKEYKC |
| 114) | KVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYT |
| | QKSLSLSPGK |

In certain embodiments, a SPINK9 polypeptide comprises, consists of, or consists essentially of an amino acid sequence selected from the group consisting of SEQ ID NOS: 55-114 as shown in Table 7 above, wherein 1 to 9 contiguous amino acids are deleted from the N terminal end of the mutant SPINK9 domain (e.g., 1 to 9 contiguous amino acids selected from IECAKQTKQ).

In certain embodiments, a SPINK9 polypeptide comprises, consists of, or consists essentially of an amino acid sequence selected from the group consisting of SEQ ID NOS: 55-114 as shown in Table 7 above, wherein 1 to 3 contiguous amino acids are deleted from the N terminal end of the mutant SPINK9 domain (e.g., 1 to 3 amino acids selected from IEC).

In certain embodiments, a SPINK9 polypeptide comprises, consists of, or consists essentially of an amino acid sequence selected from the group consisting of SEQ ID NOS: 55-114 as shown in Table 7 above, wherein the first three amino acids are deleted from the N terminal end of the mutant SPINK9 domain (e.g., the amino acids IEC).

In some preferred embodiments, a SPINK9 polypeptide comprises, consists of, or consists essentially of an amino acid sequence selected from the group consisting of SEQ ID NOS: 137-139 as shown in Table 8 below.

TABLE 8
Exemplary mutant SPINK9 domain-containing Polypeptides

| | |
|---|---|
| Spink9- | AKQTKQMVDCSHYKKLPPEQELYCTREYDPICGSDGKTYGNDCFFCSKVKKTDGTLKFVH |
| 112.K62 | FGKCEPKSCDKTHTCPPCPAPEAAGGPSVFLFPPKPKDTLMISRTPEVTCVVVDVSHEDPEV |
| G.FcΔ | KFNWYVDGVEVHNAKTKPREEQYNSTYRVVSVLTVLHQDWLNGKEYKCKVSNKALPAPI |
| (SEQ ID | EKTISKAKGQPREPQVYTLPPSREEMTKNQVSLTCLVKGFYPSDIAVEWESNGQPENNYKT |
| NO: 137) | TPPVLDSDGSFFLYSKLTVDKSRWQQGNVFSCSVLHEALHSHYTQKSLSLSPGK |
| Spink9- | AKQTKQMVDCSHYKKLPPEQELYCTREYDPICGSDGKTYGNDCFFCSKVKKTDGTLKFVH |
| 112,2K62 | FGKCEPKSCDKTHTCPPCPAPEAAGGPSVFLFPPKPKDTLYITREPEVTCVVVDVSHEDPEV |
| G.FcΔ2 | KFNWYVDGVEVHNAKTKPREEQYNSTYRVVSVLTVLHQDWLNGKEYKCKVSNKALPAPI |
| (SEQ ID | EKTISKAKGQPREPQVYTLPPSREEMTKNQVSLTCLVKGFYPSDIAVEWESNGQPENNYKT |
| NO: 138) | TPPVLDSDGSFFLYSKLTVDKSRWQQGNVFSCSVMHEALHNHYTQKSLSLSPGK |
| Spink9- | AKQTKQMVDCSHYKKLPPEQELYCTREYDPICGSDGKTYGNDCFFCSKVKKTDGTLKFVH |
| 112.3K62 | FGKCEPKSCDKTHTCPPCPAPEAAGGPSVFLFPPKPKDTLMISRTPEVTCVVVDVSHEDPEV |
| G.FcΔ3 | KFNWYVDGVEVHNAKTKPREEQYNSTYRVVSVLTVLHQDWLNGKEYKCKVSNKALPAPI |
| (SEQ ID | EKTISKAKGQPREPQVYTLPPSREEMTKNQVSLTCLVKGFYPSDIAVEWESNGQPENNYKT |
| NO: 139) | TPPVLDSDGSFFLYSKLTVDKSRWQQGNVFSCSVMHEALHNHYTQKSLSLSPGK |

A SPINK9 polypeptide may also include additional modifications that impact a property of a SPINK9 polypeptide, such as, but not limited to, increasing a desirable therapeutic property (e.g., serum half-life) and providing a simple means of purification.

In some embodiments, a SPINK9 polypeptide includes one or more linkages other than peptide bonds such that at least two adjacent amino acids are joined via a linkage other than an amide bond. The use of non-peptide bonds may be used to reduce or eliminate undesired proteolysis or degradation, to increase stability in a physiological environment after administration to a subject, to increase or decrease conformational flexibility, or a combination of the foregoing. Suitable non-peptide bonds include, but are not limited to, —CH₂NH—, —CH₂S—, —CH₂CH₂—, —CH═CH— (cis and trans), —COCH₂—, —CH(OH)CH₂— and —CH₂SO—. Other alternatives include amide bioisosteres such as, but not limited to, 1,2,3-triazole, oxadiazole, imidazole, tetrazole, pyrazole, indole, pyridine, pyrazine, retroinverted and reverse amide, urea, olefin, fluoroalkene, trifluoroethylamine, amidine, ester, sulfonamide, phosphonamidate, thioamide, and carbamate (Kumari, (2020) J. Med. Chem. 63(21), 12290-12358).

In some embodiments, a SPINK9 polypeptide is modified by the addition of polyethylene glycol (PEG), a PEG mimetic, polypropylene glycol, or polyoxyalkylenes. Such modifications have been shown to increase physical and thermal stability, protect against enzymatic degradation, increase solubility, increase in-vivo circulating half-life, decrease clearance, reduce immunogenicity and antigenicity, and reduce toxicity. Suitable PEG molecules are generally soluble in water at room temperature and have the general formula R(O-CH₂—CH₂)ₙO-R, where R is hydrogen or a protective group (e.g., an alkyl or an alkanol group), and where n is an integer from 1 to 1000. A suitable PEG may be linear or branched. In some embodiments, a suitable PEG has a number average molecular weight (Mn) between 4,000 and 10,000 Daltons, although other Mn may be used. PEG may be bound to a SPINK9 polypeptide via a spacer comprising a terminal reactive group. The spacer may be, for example, a terminal reactive group that mediates a bond between the free amino or carboxyl groups of one or more amino acids and polyethylene glycol.

In some embodiments, a SPINK9 polypeptide is modified by altering the naturally occurring glycosylation pattern. Glycosylation can dramatically affect the physical properties of proteins and can also be important in protein stability, secretion, and subcellular localization. Addition of glycosylation sites can be accomplished by altering the amino acid sequence, such as substitution of a naturally occurring amino acid with one or more serine or threonine residues (for O-linked glycosylation sites) or asparagine residues (for N-linked glycosylation sites). Another means of increasing the number of carbohydrate moieties on a polypeptide is by chemical or enzymatic coupling of glycosides to the polypeptide. Removal of carbohydrates may be accomplished chemically or enzymatically, or by substitution of codons encoding amino acid residues that are/can be glycosylated. Chemical deglycosylation techniques are known, and enzymatic cleavage of carbohydrate moieties on polypeptides can be achieved by the use of a variety of endo- and exo-glycosidases.

### Nucleic Acids

Provided herein are nucleic acid molecules that encode the polypeptides described herein. The nucleic acids may be present, for example, in whole cells, in a cell lysate, or in a partially purified or substantially pure form. A nucleic acid molecule described herein can be isolated using standard molecular biology techniques and the sequence information provided herein. For example, nucleic acids corresponding to the nucleotide sequence encoding one or more of the polypeptides disclosed herein can be prepared by standard techniques known in the art.

The present disclosure provides vectors (e.g., a viral vector, such as an adenovirus-based expression vector) that contain the nucleic acid molecules described herein. A viral vector may contain additional DNA segments that may be ligated into the viral genome. Certain vectors are capable of autonomous replication in a host cell into which they are introduced (e.g., bacterial vectors having a bacterial origin of replication, or episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”). In some embodiments, provided herein are nucleic acids operable linked to one or more regulatory sequences (e.g., a promoter) in an expression vector. In some embodiments, the cell transcribes the nucleic acid provided herein and thereby expresses an antibody, antigen-binding fragment thereof, or peptide described herein. The nucleic acid molecule can be integrated into the genome of the cell, or it can be extrachromosomal.

In some embodiments, the nucleic acid vectors or recombinant adenoviruses provided herein encode a polypeptide disclosed herein or otherwise contemplated herein. In some embodiments, the nucleic acid vectors comprise nucleic acid sequences that have undergone codon optimization. In such embodiments, a coding sequence is constructed by varying the codons in each nucleic acid used to assemble the coding sequence. In general, a method to identify a nucleotide sequence that optimizes codon usages for production of a peptide comprises at least the following steps (a) through (e). In step (a), oligomers are provided encoding portions of the polypeptide containing degenerate forms of the codon for an amino acid encoded in the portions, with the oligomers extended to provide flanking coding sequences with overlapping sequences. In step (b), the oligomers are treated to effect assembly of the coding sequence for the peptide. The reassembled peptide is included in an expression system that is operably linked to control sequences to affect its expression. In step (c), the expression system is transfected into a culture of compatible host cells. In step (d), the colonies obtained from the transformed host cells are tested for levels of production of the polypeptide. In step (e), at least one colony with the highest or a satisfactory production of the polypeptide is obtained from the expression system. The sequence of the portion of the expression system that encodes the protein is determined. Further description of codon optimization is provided in U.S. Patent Publication number US2010/035768, which is incorporated by reference for such teachings.

Also provided herein are host cells, comprising the nucleic acids or the vectors described herein. The polypeptides provided herein (e.g., polypeptides comprising at least one mutant SPINK9 domain) can be isolated from cells or tissue sources by an appropriate purification scheme using standard protein purification techniques, can be produced by recombinant DNA techniques, and/or can be chemically synthesized using standard peptide synthesis techniques. The peptides described herein can be produced in prokaryotic or eukaryotic host cells by expression of nucleotides encoding a peptide(s) of the present invention. Alternatively, such peptides can be synthesized by chemical methods. Methods for expression of heterologous peptides in recombinant hosts, chemical synthesis of peptides, and in vitro translation are well known in the art and are described further in Maniatis et al., Molecular Cloning: A Laboratory Manual (1989), 2nd Ed., Cold Spring Harbor, N. Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

### Methods of Use

The present invention provides SPINK9 polypeptides, and pharmaceutically acceptable salts thereof, that are useful for treating or preventing a disease or condition characterized by aberrant kallikrein activity.

In certain aspects, the invention provides a SPINK9 polypeptide, or a pharmaceutically acceptable salt thereof, for use as a medicament.

In certain aspects, methods of treating or preventing a disease or condition characterized by aberrant kallikrein activity are provided. The method includes the step of administering to a subject in need thereof an amount of a SPINK9 polypeptide, or a pharmaceutically acceptable salt thereof, thereby treating or preventing the disease or condition characterized by aberrant kallikrein activity. By reducing kallikrein activity in the subject, the disease or condition characterized by aberrant kallikrein activity is treated. In some embodiments, a therapeutically effective amount is administered to the subject.

Alternatively, in certain aspects, a SPINK9 polypeptide disclosed herein, or a pharmaceutically acceptable salt thereof, is provided for treatment of a disease or condition characterized by aberrant kallikrein activity.

Alternatively, in certain aspects, the invention provides the use of a SPINK9 polypeptide disclosed herein, or a pharmaceutically acceptable salt thereof, for the manufacture of a medicament for use in treatment of a disease or condition characterized by aberrant kallikrein activity.

As used herein, a “disease or condition characterized by aberrant kallikrein activity” refers to any disease or condition in which it is desirable to reduce kallikrein activity (e.g., KLK5 activity). For example, it may be desirable to reduce kallikrein activity in the setting of inappropriate activation or hyperactivation of kallikrein.

In certain embodiments, the disease or condition characterized by aberrant kallikrein activity is characterized by aberrant KLK5 activity.

In certain embodiments, the disease or condition characterized by aberrant kallikrein activity is a skin disease.

In certain embodiments, the skin disease is eczema, atopic eczema, atopic dermatitis, autosomal recessive ichthyosis, ichthyosiform erythroderma (IE), psoriasis, UV-induced skin injury, rosacea, or a skin infection.

In certain embodiments, the disease or condition is Netherton Syndrome.

In certain embodiments, the disease or condition characterized by aberrant kallikrein activity is selected from the group consisting of hypersensitivity of the immune system (atopy), hyper IgE syndrome, allergies (including allergies to food and airborne agents), asthma, allergic asthma, chronic inflammation, rhinitis, conjunctivitis, angioedema, eosinophilia, eosinophilic esophagitis, growth delay, failure to thrive, trichorrhexis invaginata (TI), bacterial infections of the skin, respiratory tract infections, systemic infections and gastrointestinal disorders.

In certain embodiments, the disease or condition is cancer. The cancer may be selected from ovarian cancer, uterine cancer, colorectal cancer, bladder urothelial cancer, oral squamous cell carcinoma, breast cancer, prostate cancer, bladder cancer, cervical cancer, melanoma, head and neck cancer glioma, glioblastoma multiforme, and neuroblastoma.

### Compositions

Disclosed herein are compositions, including pharmaceutical compositions, comprising a SPINK9 polypeptide described herein, the nucleic acids described herein, the vectors described herein, and/or the cells described herein. The compositions of the present invention are useful for treating or preventing various diseases characterized by aberrant kallikrein activity (e.g., proteases of the kallikrein-related peptidase family, in particular aberrant KLK5 activity).

The pharmaceutical compositions provided herein may contain an amount of a SPINK9 polypeptide, including a therapeutically effective amount, to treat an individual in need thereof. In certain embodiments, the individual is a mammal such as a human, or a non-human animal. When administered to a human, or a non-human animal, the composition (or otherwise the polypeptide) is preferably administered as a pharmaceutical composition comprising, for example, a SPINK9 polypeptide disclosed herein and a pharmaceutically acceptable carrier. Pharmaceutically acceptable carriers are well known in the art, and except insofar as any conventional carrier is incompatible with a substance or its derivatives, such as by producing any undesirable biological effect or otherwise interacting in a deleterious manner with any other component(s) of the composition, its use is contemplated to be within the scope of this present disclosure. In preferred embodiments, when such pharmaceutical compositions are for human administration, particularly for invasive routes of administration (i.e., routes, such as injection or implantation, that circumvent transport or diffusion through an epithelial barrier), the pharmaceutical composition is pyrogen-free, or substantially pyrogen-free. The carriers can be chosen, for example, to effect delayed release of an agent, to produce a desired pH range, to stabilize a Spink9 polypeptide, to modulate absorption of a Spink9 polypeptide, to modulate a desired pharmacokinetic or pharmacodynamic property of a Spink9 polypeptide, to selectively target one or more cells, tissues or organs, or any combination of the foregoing.

Such pharmaceutically acceptable carriers include, but are not limited to, water, other liquid vehicles, solvents, diluents, dispersion media, dispersion or suspension aids, surfactants, surface active agents, isotonic agents, thickening agents, emulsifying agents, preservatives, antioxidants (such as ascorbic acid or glutathione), chelating agents, solid binders, lubricants, sugars (such as dextrans, lactose, glucose and sucrose), starches (such as corn starch and potato starch), cellulose, and its derivatives (such as sodium carboxymethyl cellulose, ethyl cellulose and cellulose acetate), oils, (such as peanut oil, cottonseed oil, safflower oil, sesame oil, olive oil, corn oil and soybean oil), glycols (such as propylene glycol), polyols (such as glycerin, sorbitol, mannitol and polyethylene glycol), esters (such as ethyl oleate and ethyl laurate), buffering agents (such as magnesium hydroxide and aluminum hydroxide, isotonic saline, Ringer's solution, phosphate buffer solutions), osmolality adjusting agents, and stabilizing agents. The choice of a pharmaceutically acceptable carrier may depend on, for example, the route of administration of the composition. The preparation of a pharmaceutical composition can be a self-emulsifying drug delivery system or a self-microemulsifying drug delivery system. The pharmaceutical composition (preparation) also can be a liposome or other polymer matrix, which can have incorporated therein, for example, a SPINK9 polypeptide disclosed herein. Liposomes, for example, which comprise phospholipids or other lipids, are nontoxic, physiologically acceptable and metabolizable carriers that are relatively simple to make and administer.

The pharmaceutical composition can be provided in a unit dosage form. The phrase “unit dosage form” refers to physically discrete units, each unit containing a predetermined amount of a composition comprising a Spink9 polypeptide, either alone or in combination one or more additional therapeutic agents, sufficient to produce the desired effect. It will be appreciated that the parameters of a unit dosage form will depend on the mode of administration, the particular Spink9 polypeptide and the effect to be achieved. Suitable doses of a Spink9 polypeptide are disclosed herein. The pharmaceutical composition can also be a solution, particularly a liquid solution, suitable for parenteral administration, particularly subcutaneous or intravenous administration. The pharmaceutical composition can also be present in a lyophilized or powder form for reconstitution with a liquid solution suitable for parenteral administration, particularly subcutaneous or intravenous administration.

A pharmaceutical composition (preparation) can be administered to a subject by any of a number of routes of administration including, for example, parenteral administration; oral administration (for example, as a component of a liposome); administration through the oral mucosa (e.g., sublingually); transdermal administration (for example as a patch applied to the skin); topical administration (for example, as a cream, ointment or spray applied to the skin); or by administration by inhalation. The phrases “parenteral administration” and “administered parenterally” as used herein means modes of administration other than enteral and topical administration, usually by injection or infusion, and includes, without limitation, subcutaneous, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. In one embodiment, the mode of administration is via subcutaneous administration. In another embodiment, the mode of administration is via intravenous administration.

Details of appropriate routes of administration and compositions suitable for same can be found in, for example, U.S. Pat. No. 11,246,906 and International Publication WO 2022/099963.

The amount of SPINK9 polypeptide that can be combined with a pharmaceutically acceptable carrier to produce a single dosage form will vary depending upon the host being treated, the particular mode of administration, and the disease or condition to be treated. In some embodiments, a SPINK9 polypeptide is present in a range of from about 1 percent to about 99 percent, from about 1 percent to about 90 percent, from about 1 percent to about 75 percent, from about 1 percent to about 50 percent, from about 1 percent to about 40 percent, from about 1 percent to about 30 percent, from about 1 percent to about 20 percent, or from about 1 percent to about 10 percent.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration comprise a Spink9 polypeptide in combination with one or more pharmaceutically acceptable carries, such as, but not limited to, sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, antifungal agents, solutes which render the formulation isotonic with the blood of the intended recipient or suspending or thickening agents.

These compositions may also contain preservatives, wetting agents, emulsifying agents and dispersing agents. Prevention of the action of microorganisms may be ensured by the inclusion of various antibacterial and, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug after administration, for example subcutaneous administration. This may be accomplished through the use of depot injections to release a Spink9 polypeptide disclosed herein over a defined period of time. Depot injections are usually either solid- or oil-based and generally comprise at least one of the pharmaceutically acceptable carriers described herein. In certain embodiments, injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue. One of ordinary skill in the art is familiar with possible formulations and uses of depot injections.

Methods of administration may also be provided by implantable or biodegradable devices.

After a pharmaceutical composition has been formulated, it can be stored in sterile containers as a solution, suspension, gel, emulsion, solid, or dehydrated or lyophilized powder. Such formulations can be stored either in a ready-to-use form, a lyophilized form requiring reconstitution prior to use, a liquid form requiring dilution prior to use, or other acceptable form. Preferably, the pharmaceutical composition is provided in a single-use container. Any drug delivery apparatus can be used to deliver the pharmaceutical composition.

The present disclosure includes the use of pharmaceutically acceptable salts of a Spink9 polypeptide in the compositions and methods of the present invention. The present disclosure also includes the use of a buffer salt in a composition comprising a Spink9 polypeptide in the compositions and methods of the present invention. In certain embodiments, contemplated salts of the invention include, but are not limited to, citrate, acetate, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. Suitable salts for use with therapeutic polypeptides are known in the art (Sikora et al., (2020) Pharmaceuticals, 13, 442). In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Useful dosages of a SPINK9 polypeptide disclosed herein can be determined, at least initially, by comparing their in vitro activity and in vivo activity in animal models. Methods for the extrapolation of effective dosages in mice, and other animals, to humans are known in the art. The amount of a SPINK9 polypeptide may be varied so as to obtain an amount that is effective to achieve the desired therapeutic response for a particular subject, composition, and mode of administration, without being toxic to the subject.

The dose of a SPINK9 polypeptide administered will depend upon a variety of factors including the activity of the particular polypeptide, the route of administration, the time of administration, severity of the disease, the rate of excretion, the duration of the treatment, other active agents administered in combination, the age, sex, weight, condition, general health and prior medical history of the subject being treated, and like factors well known in the medical arts.

A physician having ordinary skill in the art can determine and prescribe the amount of the pharmaceutical composition required. For example, the physician could start doses of the pharmaceutical composition or compound at levels lower than that required in order to achieve the desired therapeutic effect and gradually increase the dosage until the desired effect is achieved. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In certain embodiments, the pharmaceutical compositions for administration, particularly subcutaneous administration and intravenous administration, comprise a dose of a Spink9 polypeptide of: about 0.1 mg to about 50 mg; about 0.1 mg to about 40 mg; about 0.1 mg to about 30 mg; about 0.1 mg to about 20 mg; about 0.1 mg to about 15 mg; about 0.1 mg to about 10 mg; about 0.1 mg to about 8 mg; about 0.1 mg to about 6 mg; about 0.1 mg to about 4 mg; about 0.1 mg to about 2 mg; about 0.1 mg to about 1 mg; about 0.5 mg to about 50 mg; about 0.5 mg to about 40 mg; about 0.5 mg to about 30 mg; about 0.5 mg to about 20 mg; about 0.5 mg to about 15 mg; about 0.5 mg to about 10 mg; about 0.5 mg to about 8 mg; about 0.5 mg to about 6 mg; about 0.5 mg to about 4 mg; about 0.5 mg to about 2 mg; about 1 mg to about 50 mg; about 1 mg to about 40 mg; about 1 mg to about 30 mg; about 1 mg to about 20 mg; about 1 mg to about 15 mg; about 1 mg to about 10 mg; about 1 mg to about 8 mg; about 1 mg to about 6 mg; about 1 mg to about 4 mg; about 1 mg to about 2 mg; about 5 mg to about 50 mg; about 5 mg to about 40 mg; about 5 mg to about 30 mg; about 5 mg to about 25 mg; about 5 mg to about 20 mg; about 5 mg to about 15 mg; about 5 mg to about 10 mg; about 5 mg to about 8 mg; about 10 mg to about 50 mg; about 10 mg to about 40 mg; about 10 mg to about 30 mg; about 10 mg to about 25 mg; about 10 mg to about 20 mg; or about 10 mg to about 15 mg.

A dose of a Spink9 polypeptide to be administered, particularly for subcutaneous administration and intravenous administration, may also be expressed in terms of μg/kg. Exemplary doses of a Spink9 polypeptide in terms of μg/kg range from: about 1 μg/kg to about 500 μg/kg; about 1 μg/kg to about 250 μg/kg; about 1 μg/kg to about 100 μg/kg; about 1 μg/kg to about 50 μg/kg; about 1 μg/kg to about 25 μg/kg; about 1 μg/kg to about 15 μg/kg; about 1 μg/kg to about 10 μg/kg; about 1 μg/kg to about 5 μg/kg; about 5 μg/kg to about 500 μg/kg; about 5 μg/kg to about 250 μg/kg; about 5 μg/kg to about 100 μg/kg; about 5 μg/kg to about 50 μg/kg; about 5 μg/kg to about 25 μg/kg; about 5 μg/kg to about 15 μg/kg; about 5 μg/kg to about 10 μg/kg; about 15 μg/kg to about 500 μg/kg; about 15 μg/kg to about 250 μg/kg; about 15 μg/kg to about 100 μg/kg; about 15 μg/kg to about 50 μg/kg; about 15 μg/kg to about 25 μg/kg; about 50 μg/kg to about 500 μg/kg; about 50 μg/kg to about 250 μg/kg; about 50 μg/kg to about 100 μg/kg; about 150 μg/kg to about 500 μg/kg; about 150 μg/kg to about 250 μg/kg; about 200 μg/kg to about 500 μg/kg; about 250 μg/kg to about 350 μg/kg; about 300 μg/kg to about 500 μg/kg; about 300 μg/kg to about 400 μg/kg; about 400 μg/kg to about 500 μg/kg. Further, a dose in μg/kg may be calculated by dividing a fixed dose as described herein by 60-70 kg for an adult subject or 15-50 kg for a pediatric subject.

In certain embodiments, a Spink9 polypeptide, either alone or as a part of a pharmaceutical composition, is administered according to a course of treatment. A course of treatment may extend for a period of 1 week, 2 weeks, 3, weeks, 1 month, 2 months, three months, 4 months, 5 months, 6 months, 7 months, 8 months, 9 months, 10 months, 11 months, 12 months, 16 months, 24 months, 36 months, 48 months, 60 months or longer. In certain embodiments, the course of treatment is for the remainder of the life of the subject.

A subject may receive a single course of treatment or multiple (i.e., 1, 2, 3, 4, 5, or more) courses of treatment. When a subject receives multiple courses of treatment, such multiple courses of treatment may be consecutive (for example, a second course of treatment is initiated immediately after the first course of treatment is concluded) or such multiple courses of treatments may be non-consecutive and separated by a non-treatment interval (for example, a third course of treatment is initiated 1 month after the second course of treatment is completed). Combinations of consecutive and non-consecutive courses of treatment may also be used (for example, a third course of treatment is initiated 1 month after the second course of treatment is completed, and the second course of treatment is initiated immediately after the first course of treatment is concluded). In certain aspects, the non-treatment interval is 1 week, 2 weeks, 3 weeks, 4 weeks, 2 months, 3 months, 4 months, 5 months, 6 months, 7 months, 8 months, 9 months, 10 months, 11 months, 12 months, or longer.

A dosing regimen may be used with a course of treatment. When multiple courses of treatment are administered to a subject, the dosing regimen may be the same for each course of treatment or the dosage regimen may be different for one or more courses of treatment. In one embodiment, a dosing regimen comprises administering a Spink9 polypeptide during a course of treatment as follows: about every 2 days, about every 3 days, about every 4 days, about every 5 days, about every 6 days, about every 7 days, about every 8 days, about every 9 days, about every 10 days, about every 11 days, about every 12 days, about every days 13 days, about every 14 days, about every 15 days, about every 16 days, about every 17 days, about every 18 days, about every 19 days, about every 20 days, about every 21 days, about every 22 days, about every 23 days, about every 24 days, about every 25 days, about every 26 days, about every 27 days, about every 28 days, about every 29 days, about every 30 days, about every 45 days, about every 60 days, about every 75 days, about every 90 days, about every 105 days, or about every 120 days.

Preferably, a subject receives multiple courses of treatment. In certain embodiments, the Spink9 polypeptide is administered by a parenteral route of administration. In certain embodiments, the Spink9 polypeptide is administered by subcutaneous administration. In certain embodiments, the Spink9 polypeptide is administered by intravenous administration.

When a subject receives a course of treatment by subcutaneous administration, a Spink9 polypeptide may be injected at the same site on the subject for each dose of a dosing regimen (for example, the upper arm) or injected at different site on the subject for one or more doses of a dosing regimen (for example, the upper arm and the abdomen). In certain embodiments, subcutaneous administration is accomplished through the use of an implantable device.

In certain embodiments, a SPINK9 polypeptide disclosed herein may be used alone or conjointly administered with another type of therapeutic agent, for example, other agents that are useful for treating or preventing a disease or condition characterized by aberrant kallikrein activity. A SPINK9 polypeptide disclosed herein can also be administered in combination with other therapeutic agents, for example, other agents that are useful for treating or preventing a disease or condition characterized by aberrant KLK5 activity. In certain embodiments, a SPINK9 polypeptide disclosed herein can also be administered in combination with one or more inhibitors of a KLK family member, particularly a KLK family member downstream of KLK5 (such as, but not limited to, KLK7 and KLK14). In certain embodiments, a SPINK9 polypeptide disclosed herein can also be administered in combination with one or more other therapeutic agents that are useful for treating or preventing Netherton Syndrome. For Example, and without being bound by theory or methodology, “conjoint administration” may refer to any form of administration of a SPINK9 polypeptide disclosed herein with other therapeutic agents such that the SPINK9 polypeptide or other therapeutic agent is administered while the previously administered agent (e.g., the SPINK9 polypeptide or the other therapeutic agent) is still effective in the body (e.g., the SPINK9 polypeptide and other therapeutic agent are simultaneously effective in the patient, which may include synergistic effects). Said SPINK9 polypeptides and other different therapeutic compounds can be administered either in the same formulation or in separate formulations, either concomitantly or sequentially. Thus, an individual who receives such treatment can benefit from a combined effect of different therapeutic agents.

### EXAMPLES

The invention now being generally described, it will be more readily understood by reference to the following examples, which are included merely for purposes of illustration of certain aspects and embodiments of the present invention, and are not intended to limit the invention.

### Example 1: Construction of Vectors and Expression of SPINK9 Polypeptides

The SPINK9 polypeptides were recombinantly generated as follows. First, cDNA sequences encoding desired SPINK9 domains (e.g., amino acids 20 to 86 of SEQ ID NO: 1) fused to a C-terminal IgG Fc fragment were designed. The codon usage was optimized for mammalian expression systems with the GeneArt codon optimizer, and their suggested Kozak sequence was used. The cDNAs were synthesized and nucleotide sequences were confirmed. The cDNAs were cloned to pcDNA3.4 expression vector using EcoRI and HindIII sites. The pcDNA3.4 vector uses the full-length human cytomegalovirus (CMV) immediate-early promoter for high level gene expression together with the woodchuck posttranscriptional regulatory element (WPRE) downstream of the cloning site to enhance transcript expression. A mouse IgG heavy chain signal peptide (MGWSCIILFLVATATGVHS) or the wild-type SPINK9 signal peptide (e.g., amino acids 1-19 of SEQ ID NO: 1) was added at the N-terminus of the SPINK9 polypeptides to ensure secretion into the cell medium. During the translocation, the signal peptide was cleaved off. Expi293 cells (A14527, ThermoFisher) or CHO-S cells (R80007, ThermoFisher) were used to express the SPINK9 polypeptides. For expression, Expi293 cells were transiently transfected with the expression vectors and the cell culture was collected. SPINK9 polypeptides were purified from the cell culture medium using a HiTrap Protein G HP 5 ml column (17-0405-01, GE Healthcare). The filtered cell culture media was loaded on the protein G column according to the protein G protocol, using PBS to equilibrate the column (binding buffer) and 0.7% acetic acid (elution buffer) to elute the SPINK9 polypeptides. The elution was monitored by measuring absorbance at 280 nm. After purification, the SPINK9 polypeptides were stored in PBS and hence directly after the elution the buffer was exchanged, either by dialysis or by desalting columns with an appropriate molecular weight cut-off. The SPINK9 polypeptides were concentrated to a concentration of 1 mg/ml with Amicon ultra centrifugal filters (Merck Millipore) with a 50 kDa molecular weight cut-off. The protein concentration was measured with a microvolume spectrophotometer using the A280 setting. The expression of the desired SPINK9 polypeptides was further confirmed by SDS page and Western blot analysis under reduced and non-reduced conditions.

### Example 2: Evaluation of Inhibitory Activity of SPINK9 Polypeptides on KLK5

The effect of SPINK9 polypeptides on KLK5 enzymatic activity was assessed. The assay was performed by incubation of 20 nM of KLK5 in 100 mM NaH₂PO₄, pH 7.5 and pH 5.5, 1 mM CHAPS with SPINK9 polypeptides (in half log dilution series from 0.3 nM to 300 nM) or the assay buffer as control at 22° C. for 10 minutes. Then reactions were initiated by addition of 100 μM of substrate BOC-VPR-AMC (R&D Systems, Minneapolis Minnesota) in a total reaction volume of 100 μL. After incubation for 15 minutes, the plate was excited at 340 nm and fluorescent emission was measured at 460 nM with a Synergy Neo microplate reader (BioTek®, Winooski, Vermont). The 50% of maximal inhibitory concentration (IC₅₀) was calculated by nonlinear regression analysis using the four parameter, sigmoidal, dose response equation using Prism GraphPad (GraphPad Software, San Diego, California). The results are shown in Table 9. Wild Type Spink9 (SEQ ID NO: 2) binding to KLK5 exhibited an IC₅₀ of 80 nM and 685 nM at pH 7.5 and 5.5, respectively.

TABLE 9
Inhibitory Effect of SPINK9 Polypeptides
on KLKS Enzymatic Activity

| | KLK5 | KLK5 |
|---|---|---|
| | IC₅₀ nM* | IC₅₀ nM* |
| SPINK9 Polypeptide | pH 7.5 | pH 5.5 |
| Spink9-30.Fc (SEQ ID NO: 55) | 2.2 | 2.4 |
| Spink9-112.K62G.Fc (SEQ ID NO: 56) | 1.1 | 0.9 |
| Spink9-183.Fc (SEQ ID NO: 57) | 1.4 | 3.0 |
| Spink9-186.Fc (SEQ ID NO: 58) | 1.9 | 8.3 |
| Spink9-189.Fc (SEQ ID NO: 59) | 1.5 | 1.8 |
| Spink9-236.Fc (SEQ ID NO: 60) | 1.5 | 3.5 |
| Spink9-112.Fc (SEQ ID NO: 61) | 1.4 | N.D. |
| Spink9-194.Fc (SEQ ID NO: 62) | 3.1 | N.D. |
| Spink9-245.Fc (SEQ ID NO: 63) | 1.7 | N.D. |
| Spink9-20.Fc (SEQ ID NO: 64) | 3.9 | N.D. |
| Spink9-21.Fc (SEQ ID NO: 65) | 1.9 | N.D. |
| Spink9-73.Fc (SEQ ID NO: 66) | 1.3 | N.D. |
| Spink9-211.Fc (SEQ ID NO: 67) | 1.3 | N.D. |
| Spink9-212.Fc (SEQ ID NO: 68) | 3.9 | N.D. |
| Spink9-237.Fc (SEQ ID NO: 69) | 1.2 | N.D. |
| Spink9-3.Fc (SEQ ID NO: 70) | 1.3 | N.D. |
| Spink9-162.Fc (SEQ ID NO: 71) | 1.2 | N.D. |
| Spink9-193.Fc (SEQ ID NO: 72) | 1.3 | N.D. |
| Spink9-202.Fc (SEQ ID NO: 73) | 1.4 | N.D. |
| Spink9-74.Fc (SEQ ID NO: 74) | 19.5 | N.D. |
| Spink9-112.K62G.FcA (SEQ ID NO: 138) | 1.3 | N.D. |

*n = 1; N.D. = not determined

### Example 3: Evaluation of Inhibitory Activity of SPINK9 Polypeptides on KLK7 and KLK14

The effect of SPINK9 polypeptides on KLK7 and KLK14 enzymatic activity was assessed. Both rhKLK7 and rhKLK14 were acquired as zymogens and each required activation with thermolysin. rhKLK7 or rhKLK14 was diluted to 200 mg/mL in activation buffer (50 mM Tris, 10 mM CaCl₂, 150 mM NaCl, 0.05% (w/v) Brij-35, pH 7.5) and mixed with same volume of 20 mg/mL of thermolysin in the same buffer. Activation was carried out at 37° C. for 1 h (rhKLK14) or 2 h (rhKLK7) and the reaction was stopped by addition of 50 mM EDTA.

The KLK14 assay was performed as described in Example 2 above except that the concentration of active KLK14 was 0.2 mg/ml. Enzymatic activity was measured by fluorescent emission and the IC₅₀ was calculated as described in Example 2 above.

The KLK7 assay was performed by incubation of 2 mg/mL of active KLK7 in 50 mM Tris, 150 mM NaCl, 0.05% (w/v) Brij-35, pH 8.0 with SPINK9 polypeptides in half log dilution (in half log dilution series from 0.3 nM to 300 nM) or assay buffer as control at 22° C. for 15 minutes. The reaction was initiated by addition of 20 mM of substrate MCA-Arg-Pro Lys-Pro-Val-Glu-Nval-Trp-Arg-Lys(Dnp)-NH₂ (R&D Systems) in a total reaction volume of 100 mL. After incubation for 15 minutes, the plate was excited at 320 nm and fluorescent emission was measured at 405 nM with a Synergy Neo microplate reader (BioTek®, Winooski, Vermont). The IC₅₀ was calculated as described in Example 2 above.

The results are shown in Table 10 for both KLK7 and KLK14.

TABLE 10
Inhibitory Effect of SPINK9 Polypeptides
on KLK7 and KLK14 Enzymatic Activity

| | KLK7 | KLK14 |
|---|---|---|
| | IC₅₀ (nM) | IC₅₀ (nM)* |
| SPINK9 Polypeptide | pH 7.5 | pH 8.0 |
| Spink9-30.Fc (SEQ ID NO: 55) | 10.8 | 0% |
| Spink9-112.K62G.Fc (SEQ ID NO: 56) | 2.4 | 246 |
| Spink9-183.Fc (SEQ ID NO: 57) | 5.4 | 0% |
| Spink9-186.Fc (SEQ ID NO: 58) | 8.3 | 0% |
| Spink9-189.Fc (SEQ ID NO: 59) | 35.3 | 0% |
| Spink9-236.Fc (SEQ ID NO: 60) | 43.6 | 0% |

*The results are presented as IC₅₀ or percent inhibition at 316 nM SPINK9 polypeptide

### Example 4: Evaluation of Human Serum Stability

An exemplary SPINK9 polypeptide, SPINK9-112.2K62G.Fc (SEQ ID NO: 76) was diluted in freshly collected human serum (1:9), and was apportioned into 200 μL aliquots. Five aliquots of the samples were then kept in an incubator set to 37° C., 5% CO₂. Samples were collected and snap frozen on day 0, day 1, day 4, day 7, and day 14, and then kept at −80° C. until analysis. The binding activity of the tested samples to recombinant human KLK5 C-6His (Novoprotein, Catalog No. C415) was evaluated by ELISA as described below.

The binding of SPINK9-112.2K62G.Fc after serum incubation was determined by ELISA. Plates were pre-coated with 100 μL/well of THE™ His tag mAb (2 μg/mL) (Genscript; Catalog No: A00186) overnight in a refrigerator set to 4° C. After blocking with 200 μL/well of 0.5% casein for 1 hour, the plates were washed 3 times with 1×PBST. Then 0.25 μg/ml of recombinant human KLK5 C-6His was added into the plate in a volume of 100 μL/well and incubated for 1 hour at ambient temperature. After washing the plates 3 times with 1×PBST, various concentrations (20, 4, 0.8, 0.16, 0.032, 0.0064, 0.0013, 0.00026, 0.000051 and 0.000010 nM) of SPINK9-112.2K62G.Fc with dilution buffer (0.25% casein) were added to the plates in a volume of 100 μL/well and incubated for 2 hours at ambient temperature. After washing the plates 3 times with 1×PBST, 100 μL/well of HRP-labeled Goat anti-Human IgG antibody (1:5000) (Bethyl; Catalog No: A80-304P-16) with the dilution buffer (0.25% casein) were added into each well and incubated for 1 hour at ambient temperature. After washing 6 times with 1×PBST, the color was developed by dispensing 100 μL/well of TMB substrate (Life Technologies; Catalog No: 00203) for 4 minutes, and the reaction was stopped by adding 100 μL/well of 2M HCl. The absorbance was read at 450 and 540 nm using a microplate reader (SpectraMax M5e). The EC₅₀ values were calculated by four-parameter non-linear regression analysis using GraphPad Prism software.

Human serum incubated SPINK9-112.2K62G.Fc at different time points were tested for its ability to bind to recombinant human KLK5 C-6His. The results of serum stability test (Table 11) show that SPINK9-112.2K62G.Fc incubated with serum for up to two weeks maintained binding activity to KLK5.

TABLE 11
Results of human serum stability test for SPINK9-112.2K62G.Fc

| Serum-incubated SPINK9- | Antigen: 0.25 μg/mL |
|---|---|
| 112.2K62G.Fc | recombinant human |
| (SEQ ID NO: 76) | KLK5 C-6His |
| Incubation time | EC₅₀ (nM) |
| Day 0 | 0.060 |
| Day 1 | 0.059 |
| Day 4 | 0.080 |
| Day 7 | 0.11 |
| Day 14 | 0.065 |

Table 12 shows human serum stability and affinity tests for other Spink9 polypeptides performed according to the procedures described above.

TABLE 12
Results of human serum stability test for Spink9 polypeptides

| | | Spink9- | | | | |
|---|---|---|---|---|---|---|
| | Spink9-30 | 112.K62G | Spink9-183 | Spink9-186 | Spink9-189 | Spink9-236 |
| | (SEQ ID | (SEQ ID | (SEQ ID | (SEQ ID | (SEQ ID | (SEQ ID |
| | NO: 55) | NO: 56) | NO: 57) | NO: 58) | NO: 59) | NO: 60) |
| Day | EC₅₀ (nM) | EC₅₀ (nM) | EC₅₀ (nM) | EC₅₀ (nM) | EC₅₀ (nM) | EC₅₀ (nM) |
| 0 | 0.08 | 0.10 | 0.07 | 0.09 | 0.06 | 0.05 |
| 1 | 0.10 | 0.14 | 0.09 | 0.16 | 0.08 | 0.08 |
| 4 | 0.10 | 0.16 | 0.12 | 0.21 | 0.10 | 0.12 |
| 7 | 0.10 | 0.17 | 0.11 | 0.12 | 0.12 | 0.10 |
| 14 | 0.13 | 0.17 | 0.10 | 0.29 | 0.16 | 0.11 |

### Example 5: Binding to Human KLK5 at 7.4 by Surface Plasmon Resonance (SPR)

The Biacore T200 (GE Healthcare) with series S CM5 sensor chip (GE Healthcare) was used to conduct the SPR analysis. The anti-human IgG Fc monoclonal antibody was diluted to 25 μg/ml in immobilization buffer (10 mM sodium acetate, pH 5.0).

The CM5 sensor chip was activated for 420 s at a flow rate of 10 μL/min with the activator mixture (prepared by mixing 400 mM EDC and 100 mM NHS immediately prior to injection). 25 μg/mL of anti-human IgG Fc monoclonal antibody in immobilization buffer was then injected to Fc2 sample channel for 420 s at a flow rate of 10 μL/min, resulting in immobilization levels of about 7000 RU. The sensor chip was deactivated by 1 M ethanolamine hydrochloride-NaOH (GE Healthcare) for 420 s at a flow rate of 10 μL/min. The reference surface channel Fc1 was prepared in the same manner.

The Spink9 polypeptides were diluted to m0.1 to 0.7 pig/mL in running buffer (1×HBS-EP; 10 mM HEPES, 150 mM NaCl, 3 mM EDTA, with 0.005% Tween-20, pH 7.4) and then injected into channel Fc2 at a flow rate of 10 μL/min to reach a capture level of about 35.6 to 64.5 RU.

The KLK5 protein (Novoprotein; Catalog No. C415) was diluted with running buffer (1×HEPES; 10 mM HEPES, 150 mM NaCl, 3 mM EDTA, with 0.005% Tween-20, pH 7.4) to 5-6 concentrations as described below. KLK5 protein was injected to Fc1 and Fc2 channels at a flow rate of 30 μL/min for an association phase of 120 s, followed by 300 s dissociation (the association and dissociation phases were all carried out in running buffer). Ascending concentrations of KLK5 analyte were analyzed. After each cycle of interaction analysis, the sensor chip surface was regenerated with 3 M MgCl as injection buffer at a flow rate of 10 μL/min for 30 s to remove the ligand and any unbound KLK5.
Spink9 WT-100, 50, 25, 12.5, and 6.25 nM KLK5
Spink9-30.Fc (SEQ ID NO: 55)—65, 32.5, 16.25, 8.125, 4.063, and 2.032 nM KLK5
Spink9-112.K62G.Fc (SEQ ID NO: 56)—50, 25, 12.5, 6.25, and 3.125 nM KLK5
Spink9-183.Fc (SEQ ID NO: 57)—100, 50, 25, 12.5, 6.25, and 3.125 nM KLK5
Spink9-186.Fc (SEQ ID NO: 58)—100, 50, 25, 12.5, 6.25, and 3.125 nM KLK5
Spink9-189.Fc (SEQ ID NO: 59)—100, 50, 25, 12.5, 6.25, and 3.125 nM KLK5
Spink9-236.Fc (SEQ ID NO: 60)—100, 50, 25, 12.5, 6.25, and 3.125 nM KLK5

The sensorgrams for reference channel and buffer channel were subtracted from the test sensorgrams. The experimental data were fitted by steady state affinity model. A molecular weight of 30.65 kDa was used to calculate the molar concentration of human KLK5. Table 13 shows the K_{D} values for the tested polypeptides.

TABLE 13
Binding affinity of Spink9 polypeptides to human KLKS

| | K_{D} (M) |
|---|---|
| Spink9 WT | 9.90E−08 |
| Spink9-30 (SEQ ID NO: 55) | 9.09E−11 |
| Spink9-112.K62G (SEQ ID NO: 56) | 7.85E−14 |
| Spink9-183 (SEQ ID NO: 57) | 2.16E−11 |
| Spink9-186 (SEQ ID NO: 58) | 1.89E−11 |
| Spink9-189 (SEQ ID NO: 59) | 1.52E−10 |
| Spink9-236 (SEQ ID NO: 60) | 2.909E−11 |

### Example 6: Binding to Human FcRn at pH 6.0 and 7.4 by Surface Plasmon Resonance (SPR)

The Biacore 8K with series S CM5 sensor chip (Cytiva) was used to conduct the SPR analysis. The CM5 sensor chip was activated for 420 s at a flow rate of 10 μL/min with the activator mixture (prepared by mixing 400 mM EDC and 100 mM NHS immediately prior to injection). 10 μg/mL of Spink9-30 (SEQ ID NO: 55), Spink9-112.K62G (SEQ ID NO: 56), Spink9-183 (SEQ ID NO: 57), Spink9-186 (SEQ ID NO: 58), Spink9-189 (SEQ ID NO: 59), Spink9-236 (SEQ ID NO: 60), Spink9-112.2K62G (SEQ ID NO: 76) and Rituximab (Roche) in 10 mM NaAc (pH 5.5) was then injected to Fc2 for 60 s at a flow rate of 10 μL/min, respectively. The reference channel Fc1 was blocked. The sensor chip was deactivated by 1 M ethanolamine-HCl (Cytiva) for 420 s at a flow rate of 10 μL/min.

8 concentrations (46.875, 93.75, 187.5, 375, 750, 1500, 3000, and 6000 nM) of analyte human FcRn in 1×PBST (50 mM Na₂HPO₄/NaH₂PO₄, 150 mM NaCl, 0.05% Tween-20, pH 6.0 or pH7.4) (AcrobioSystems; Catalog No: FCM-H5286) and running buffer (1×PBST, pH 6.0) were injected to Fc1 and Fc2 at a flow rate of 30 μL/min for an association phase of 60 s, followed by 90 s dissociation. 1×PBS (pH 7.4) as regeneration buffer was injected to flow cells for 45 s at a flow rate of 10 μL/min following every dissociation phase.

The sensorgrams for reference channel and buffer channel were subtracted from the test sensorgrams. The experimental data were fitted by steady state affinity model. A molecular weight of 45 kDa was used to calculate the molar concentration of human FcRn. Table 14 shows the K_{D} values for the tested polypeptides.

TABLE 14
Binding affinity of mutant Spink9 polypeptides to human FcRn

FcRn K_{D} (M)

| | pH 6.0 | pH 7.4 |
|---|---|---|
| Spink9-30 (SEQ ID NO: 55) | 2.29E−07 | Weak |
| Spink9-112.K62G (SEQ ID NO: 56) | 2.33E−07 | Weak |
| Spink9-112.2K62G (SEQ ID NO: 76) | 1.89E−07 | Weak |
| Spink9-183 (SEQ ID NO: 57) | 2.22E−07 | Weak |
| Spink9-186 (SEQ ID NO: 58) | 2.32E−07 | Weak |
| Spink9-189 (SEQ ID NO: 59) | 2.23E−07 | Weak |
| Spink9-236 (SEQ ID NO: 60) | 2.31E−07 | Weak |
| Rituximab | 3.10E−06 | Weak/No |

### Example 7: a Single Dose PK Study of SPINK9 Polypeptides in Cynomolgus Monkeys

Eight cynomolgus monkeys (4 male and 4 female) having body weight ranging from 2.5 to 3.5 kg were used in this study. Animals were supplied with approximately 120 grams of Certified Monkey Diet daily (Beijing Vital Keao Feed Co., Ltd. Beijing, P. R. China). Monkeys received fresh fruits daily. Animals were provided reverse-osmosis purified water ad libitum by an automated watering system.

Four (4) males and four (4) females cynomolgus monkeys were randomly assigned to four (4) groups (one male and one female monkey per group) to evaluate the pharmacokinetics and pharmacodynamics of Spink9-112.K62G (SEQ ID NO: 56), Spink9-112.2K62G.Fc (SEQ ID NO: 76), Spink9-186 (SEQ ID NO: 58), and Spink9-183 (SEQ ID NO: 57). Spink9 polypeptides were administrated by a single dose of 10 mg/kg via intravenous (IV) infusion over 45 minutes in a PBS buffer. Animals in Group 1 were administered Spink9-112.K62G (SEQ ID NO: 56). Animals in Group 2 were administered Spink9-112.2K62G.Fc (SEQ ID NO: 76). Animals in Group 3 were administrated Spink9-186 (SEQ ID NO: 58). Animals in Group 4 were administrated Spink9-183 (SEQ ID NO: 57). Animals were randomly assigned to groups based on body weight. Animals were weighed prior to dose administration and dose amount was determined based on the body weights which were recorded on the dose record sheet. The start date of the first dose was recorded as Day 0.

At least 200 μL blood sample was collected from a cephalic or saphenous vein at sampling time points from the animals in all groups. For samples collected within the first hour of dosing, a ±1 minute was acceptable. For the remaining time points, samples that were taken within 5% of the scheduled time. All blood samples were collected into commercially available tubes containing coagulant. Then, the tubes containing blood samples were maintained at room temperature for 30 minutes before centrifugation. The samples were centrifuged, and the serum was collected for PK assays. Samples were collected at the following time points: pre-dose (Day-1), and 5 min, 15 min, 30 min, 1 h, 4 h, 8 h, 12 h, 24 h (Day 1), 48 h (Day 2), 96 h (Day 4), 144 h (Day 6), 192 h (Day 8), 240 h (Day 10), 288 h (Day 12), 384 h (Day 16), 480 h (Day 20), 576 h (Day 24), and 672 h (Day 28) post-dose.

Animals were observed for body weight and eating condition (twice daily). Cage-side observations for general health and appearance were done once daily. Animals were observed before and after dosing, and at each sample collection time point. Bodyweight and eating condition were recorded. Each animal was weighed twice at acclimation period and once pretest (on Day 0). After administration of the test articles each animal was weighed twice per week from Day 0 to Day 28. Daily food consumption was also measured from Day 0 to Day 28 for all animals.

### PK Methods

The concentration of the test articles in the serum was determined using 2 different assays bioanalytical ELISA methods, the Fc+Fc assay and the KLK5+Fc assay.

Fc+Fc: Briefly, 96-well ELISA plates were incubated overnight at 4° C. with a solution of 1.0 μg/mL Goat Anti-human IgG (Southern Biotech; Catalog No:2049-01). After washing and blocking, serial diluted plasma samples were added, followed by the addition of biotin labeled Goat anti-human IgG (Dilution factor: 8000) (Southern Biotech; Catalog No:2049-01) as the detection antibody. Streptavidin-HRP (Dilution factor: 20000) (Thermo-Fischer; Catalog No: 21127) and TMB (Life Technologies; Catalog No: 002023) substrate were used for color development. The reaction was stopped after approximately 5-10 minutes through the addition of 2 M HCl. The absorbance was read at 450 nm and 540 nm using a microplate spectrophotometer (SpectraMax M5e). The OD value of the samples were used to obtain the plasma concentration of the test articles using a standard curve. The detection limit of this ELISA method was 0.391 ng/mL.
KLK5+Fc: Briefly, 96-well ELISA plates were incubated overnight at 4° C. with a solution of 1.0 μg/mL His Tag Antibody (Sino Biological; Catalog No: A00186-100). The plates were washed and 0.25 μg/mL recombinant Human KLK5 C-6His (Novoprotein; Catalog No: C415) was added in each well. After washing and blocking, serial diluted plasma samples were added, followed by biotin labeled Goat anti-human IgG (Dilution factor: 8000) (Southern Biotech; Catalog No: 2049-08). Streptavidin-HRP (Dilution factor: 20000) (Thermo-Fischer; Catalog No: 21127) and TMB (Life Technologies; Catalog No: 002023) substrate were used for color development. The reaction was stopped after approximately 5-10 minutes through the addition of 2 M HCl. The absorbance was read at 450 nm and 540 nm using a microplate spectrophotometer (SpectraMax M5e). The OD value of the samples were used to obtain the plasma concentration of the test articles using a standard curve. The detection limit of this ELISA method was 0.195 ng/mL.

The serum concentration of four test articles in monkeys was subjected to a non-compartmental pharmacokinetic analysis by using the Phoenix WinNonlin software (version 8.1, Pharsight, Mountain View, CA). The linear/log trapezoidal rule was applied in obtaining the PK parameters.

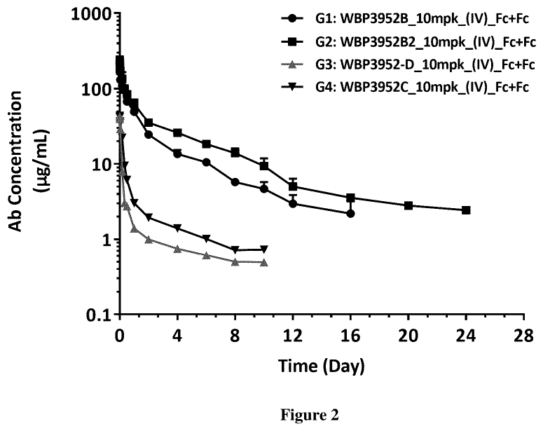

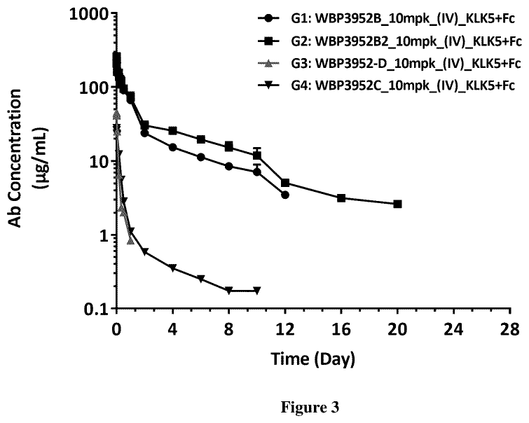

Pharmacokinetic parameters for the test articles are provided in Tables 15 and 16 for the Fc+Fc method and the KLK5+Fc method, respectively, and shown in FIGS. 2 and 3, respectively.

TABLE 15
Summary of Pharmacokinetic Parameters: Fc + Fc

| | | | | |
|---|---|---|---|---|
| | G1: | G2: | G3: | G4: |
| | Spink9- | Spink9- | Spink9- | Spink9- |
| Test Article | 112.K62G.Fc | 112.2K62G.Fc | 186.Fc | 183.Fc |

| | |
|---|---|
| | Fc + Fc |
| Method | 10 mg/kg, IV |
| Dose | infusion for 45 min |

| | | | | |
|---|---|---|---|---|
| t_{1/2} (h) | 94.5 | 128 | 137 | 101 |
| Cₘₐₓ (μg/mL) | 203 | 242 | 44.0 | 45.6 |
| AUC₀₋ₗₐₛₜ (h*μg/mL) | 5230 | 8158 | 275 | 498 |
| AUC_{0-inf} (h*μg/mL) | 5589 | 8743 | 385 | 602 |
| Cl_{obs} (mL/h/kg) | 1.81 | 1.14 | 26.8 | 16.7 |
| MRT_{0-inf} (h) | 74.0 | 101 | 27.3 | 23.8 |
| MRT₀₋ₗₐₛₜ (h) | 99.6 | 134 | 125 | 74.1 |
| Vss_{obs} (mL/kg) | 178 | 153 | 3132 | 1242 |

TABLE 16
Summary of Pharmacokinetic Parameters: KLK5 + Fc

| | | | | |
|---|---|---|---|---|
| | G1: | G2: | G3: | G4: |
| | Spink9- | Spink9- | Spink9- | Spink9- |
| Antibody | 112.K62G.Fc | 112.2K62G.Fc | 186.Fc | 183.Fc |

| | |
|---|---|
| | KLK5 + Fc |
| Method | 10 mg/kg, IV |
| Dose | infusion for 45 min |

| | | | | |
|---|---|---|---|---|
| t_{1/2} (h) | 97.8 | 131 | 10.6 | 46.8 |
| Cₘₐₓ (μg/mL) | 247 | 260 | 46.3 | 27.9 |
| AUC₀₋ₗₐₛₜ (h*μg/mL) | 6118 | 8114 | 169 | 220 |
| AUC_{0-inf} (h*μg/mL) | 6770 | 9857 | 171 | 231 |
| Cl_{obs} (mL/h/kg) | 1.48 | 1.03 | 58.5 | 43.7 |
| MRT_{0-inf} (h) | 63.5 | 90.0 | 11.0 | 15.4 |
| MRT₀₋ₗₐₛₜ (h) | 96.2 | 156 | 13.2 | 28.8 |
| Vss_{obs} (mL/kg) | 143 | 158 | 773 | 1290 |

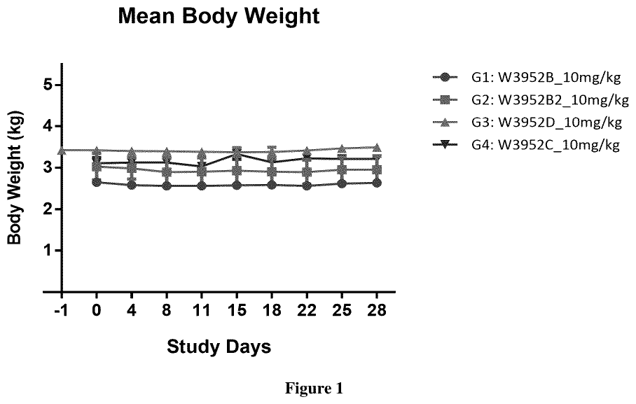

All animals behaved normally. No formulation-related effects were seen in body weight and body weight gain (FIG. 1). No formulation-related effects on food consumption were seen during the study.

The pharmacokinetic results demonstrate that Spink9-112.2K62G.Fc polypeptide exhibited the most favorable result, with an average half-life of 128 h, average AUC₀₋ₗₐₛₜ of 8158 h·μg/mL, and average Cl__{obs} of 1.14 mL/h/kg in the Fc+Fc assay and an average half-life of 131 h, an average AUC₀₋ₗₐₛₜ of 8114 h·μg/mL, and an average Cl__{obs} of 1.03 mL/h/kg in the KLK5+Fc assay.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

### INCORPORATION BY REFERENCE

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

We claim:

1. A polypeptide, comprising at least one mutant SPINK9 domain, wherein said at least one mutant SPINK9 domain comprises, in N-terminus to C-terminus order, a first amino acid sequence that shares at least 70% sequence identity to amino acids 1-20 set forth in SEQ ID NO: 2, a second amino acid sequence that comprises at least one amino acid substitution relative to amino acids 21-34 set forth in SEQ ID NO: 2, and a third amino acid sequence that shares at least 70% sequence identity to amino acids 35-67 set forth in SEQ ID NO: 2.

2. The polypeptide of claim 1, wherein the polypeptide binds to and inhibits an active kallikrein-related peptidase.

3. The polypeptide of claim 1 or 2, wherein the polypeptide inhibits the activity of kallikrein-related peptidase 5 (KLK5).

4. The polypeptide of any one of claims 1 to 3, wherein the second amino acid sequence comprises at least one amino acid substitution relative to positions 22, 24, 25, 26, 28, 29, 30 of SEQ ID NO: 2, or any combination thereof, and optionally one or more amino acid changes relative to positions 21, 23, 27, 31, 32, 33, and 34 of SEQ ID NO: 2.

5. The polypeptide of any one of claims 1 to 3, wherein the first amino acid sequence optionally comprises the amino acids at positions 1, 2, and 3 of SEQ ID NO: 2, the second amino acid sequence comprises at least one amino acid substitution relative to positions 22, 24, 25, 26, 28, 29, 30 of SEQ ID NO: 2, or any combination thereof, and optionally one or more amino acid changes relative to positions 21, 31, 32, 33, and 34 of SEQ ID NO: 2.

6. The polypeptide of any one of claims 1 to 3, wherein the first amino acid sequence comprises a deletion of the amino acids at positions 1, 2, and 3 of SEQ ID NO: 2, the second amino acid sequence comprises at least one amino acid substitution relative to positions 22, 24, 25, 26, 28, 29, 30 of SEQ ID NO: 2, or any combination thereof, and optionally one or more amino acid changes relative to positions 21, 31, 32, 33, and 34 of SEQ ID NO: 2.

7. The polypeptide of any one of claims 1 to 6, wherein the second amino acid sequence comprises a substitution of a Glu, Met, Gln, Ala, Leu, Val, Asp, Asn, Ser, Lys, or His amino acid for the amino acid relative to position 22 of SEQ ID NO: 2 or the amino acid at position 22 of SEQ ID NO: 2 is Gly.

8. The polypeptide of claim 7, wherein the second amino acid sequence comprises a substitution of a Glu, Thr, Val, Leu, Ala, Ile, Phe, Ser, or Tyr amino acid for the amino acid relative to position 24 of SEQ ID NO: 2 or the amino acid at position 24 of SEQ ID NO: 2 is Gln.

9. The polypeptide of claim 7 or 8, wherein the second amino acid sequence comprises a substitution of a Leu, Ile, His, Glu, Trp, Ser, Ala, Val, Tyr, Asp, or Met amino acid for the amino acid relative to position 25 of SEQ ID NO: 2 or the amino acid at position 25 of SEQ ID NO: 2 is Arg.

10. The polypeptide of any one of claims 7 to 9, wherein the second amino acid sequence comprises a substitution of a Tyr, Trp, Met, His, Lys, or Asn amino acid for the amino acid relative to position 26 of SEQ ID NO: 2 or the amino acid at position 26 of SEQ ID NO: 2 is Phe.

11. The polypeptide of any one of claims 7 to 10, wherein the second amino acid sequence comprises a substitution of a Thr, Ser, or Gly amino acid for the amino acid relative to position 28 of SEQ ID NO: 2 or the amino acid at position 28 of SEQ ID NO: 2 is His.

12. The polypeptide of any one of claims 7 to 11, wherein the second amino acid sequence comprises a substitution of a Arg or Lys amino acid for the amino acid relative to position 29 of SEQ ID NO: 2 or the amino acid at position 29 of SEQ ID NO: 2 is His.

13. The polypeptide of any one of claims 7 to 12, wherein the second amino acid sequence comprises a substitution of a Glu or Asp amino acid for the amino acid relative to position 30 of SEQ ID NO: 2 or the amino acid at position 30 of SEQ ID NO: 2 is Met.

14. The polypeptide of any one of claims 1 to 6, wherein the second amino acid sequence comprises a substitution of a Glu or Met amino acid for the amino acid relative to position 22 of SEQ ID NO: 2.

15. The polypeptide of claim 14, wherein the second amino acid sequence comprises a substitution of a Glu, or Thr amino acid for the amino acid relative to position 24 of SEQ ID NO: 2.

16. The polypeptide of claim 14 or 15, wherein the second amino acid sequence comprises a substitution of a Leu, Ile, His, Glu, or Trp amino acid for the amino acid relative to position 25 of SEQ ID NO: 2.

17. The polypeptide of any one of claims 14 to 16, wherein the second amino acid sequence comprises a substitution of a Tyr or Trp amino acid for the amino acid relative to position 26 of SEQ ID NO: 2.

18. The polypeptide of any one of claims 14 to 17, wherein the second amino acid sequence comprises a substitution of a Thr, Ser, or Gly amino acid for the amino acid relative to position 28 of SEQ ID NO: 2.

19. The polypeptide of any one of claims 14 to 18, wherein the second amino acid sequence comprises a substitution of a Arg or Lys amino acid for the amino acid relative to position 29 of SEQ ID NO: 2.

20. The polypeptide of any one of claims 14 to 19, wherein the second amino acid sequence comprises a substitution of a Glu or Asp amino acid for the amino acid relative to position 30 of SEQ ID NO: 2.

21. The polypeptide of any one of claims 7 to 20, wherein the third amino acid sequence comprises a substitution of a Gly amino acid for the amino acid relative to position 43 of SEQ ID NO: 2

22. The polypeptide of any one of claims 7 to 21, wherein the second amino acid sequence comprises a substitution of Thr-Arg-Glu; Thr-Arg-Asp; Thr-Lys-Glu; Thr-Lys-Asp; Ser-Arg-Glu; or Gly-Arg-Asp amino acids for the amino acid relative to position 28-30 of SEQ ID NO: 2.

23. The polypeptide of any one of claims 1 to 3, wherein the at least one mutant SPINK9 domain comprises an amino acid sequence selected from SEQ ID NOS: 4-23 or 115-134.

24. The polypeptide of any one of claims 1 to 22, wherein the polypeptide comprises from two to twenty mutant SPINK9 domains.

25. The polypeptide of claim 24, where each of the two to twenty mutant SPINK9 domains comprises an independently selected amino acid sequence of any one of SEQ ID NOS: 4-23 or 115-134.

26. The polypeptide of any one of claims 1 to 22, wherein the polypeptide comprises a first and a second independently selected mutant SPINK9 domain; and each of the first and the second mutant SPINK9 domains comprise an independently selected amino acid sequence of any one of SEQ ID NOS: 4-23 or 115-134.

27. The polypeptide of claim 26, wherein the first and the second mutant SPINK9 domains are identical.

28. The polypeptide of claim 26, wherein the first and the second mutant SPINK9 domains are not identical.

29. The polypeptide of any one of claims 1 to 22, wherein the polypeptide comprises a first, a second, and a third independently selected mutant SPINK9 domain; and each of the first, the second, and the third mutant SPINK9 domains comprise an independently selected amino acid sequence of any one of SEQ ID NOS: 4-23 or 115-134.

30. The polypeptide of claim 29, wherein each of said mutant SPINK9 domains are identical.

31. The polypeptide of claim 29, wherein each of said mutant SPINK9 domains are not identical.

32. The polypeptide of any one of claims 1 to 22, wherein the polypeptide comprises a first polypeptide chain and a second polypeptide chain linked through a Fc portion, wherein the first polypeptide chain comprises a first and a second mutant SPINK9 domain and the second polypeptide chain comprises a third and a fourth mutant SPINK9 domain and each of the first, second, third, and fourth mutant SPINK9 domains comprise an independently selected amino acid sequence of SEQ ID NO: 4-23 or 115-134.

33. The polypeptide of claim 32, wherein each of said mutant SPINK9 domains are identical.

34. The polypeptide of claim 32, wherein each of said mutant SPINK9 domains are not identical.

35. The polypeptide of any one of claims 24 to 34, wherein adjacent mutant SPINK9 domains are linked by direct fusion, a linker sequence, or any combination thereof.

36. The polypeptide of claim 35, wherein at least one mutant SPINK9 domain is linked to an adjacent mutant SPINK9 domain by a linker sequence.

37. The polypeptide of claim 35, wherein at least one mutant SPINK9 domain is linked to an adjacent mutant SPINK9 domain by direct fusion.

38. The polypeptide of any one of claims 35 to 37, wherein the linker sequence comprises a cleavage site.

39. The polypeptide of claim 38, wherein the cleavage site is recognized by a subtilisin-like proprotein convertase.

40. The polypeptide of any one of claims 1 to 22, wherein the polypeptide comprises a first polypeptide chain and a second polypeptide chain linked through a Fc portion, wherein the first polypeptide chain comprises a first mutant SPINK9 domain and the second polypeptide chain comprises a second mutant SPINK9 domain and each of the first and second mutant SPINK9 domains comprise an independently selected amino acid sequence of SEQ ID NOS: 4-23 or 115-134.

41. The polypeptide of claim 40, wherein the first and the second mutant SPINK9 domains are identical.

42. The polypeptide of claim 40, wherein the first and the second mutant SPINK9 domains are not identical.

43. A polypeptide, comprising at least one mutant SPINK9 domain, wherein said at least one mutant SPINK9 domain comprises an amino acid sequence represented by Formula I; Ile Glu Cys Ala Lys Gln Thr Lys Gln Met Val Asp Cys Ser His Tyr Lys Lys Leu Pro Pro X₁ Gln X₂ X₃ X₄ Cys X₅ X₆ X₇ Tyr Asp Pro Ile Cys Gly Ser Asp Gly Lys Thr Tyr X₈ Asn Asp Cys Phe Phe Cys Ser Lys Val Lys Lys Thr Asp Gly Thr Leu Lys Phe Val His Phe Gly Lys Cys (SEQ ID NO: 3);
provided that the amino acid sequence represented by Formula I is not SEQ ID NO: 2.

44. The polypeptide of claim 43, wherein the polypeptide binds to and inhibits an active kallikrein-related peptidase.

45. The polypeptide of claim 43 or 44, wherein the polypeptide inhibits the activity of kallikrein-related peptidase 5 (KLK5).

46. The polypeptide of claim 45, wherein the inhibition is selective for or specific to KLK5.

47. The polypeptide of any one of claims 43 to 46, wherein X₁ is selected from: i) Gly, Glu, Met, Gln, Ala, Leu, Val, Asp, Asn, Ser, Lys, or His; ii) Gly, Glu, or Met; iii) Glu, Met, Gln, Ala, Leu, Val, Asp, Asn, Ser, Lys, or His; or iv) Glu or Met.

48. The polypeptide of any one of claims 43 to 47, wherein X₂ is selected from: i) Gln, Glu, Thr, Val, Leu, Ala, Ile, Phe, Ser, or Tyr; ii) Gln, Glu, or Thr; iii) Glu, Thr, Val, Leu, Ala, lie, Phe, Ser, or Tyr; or iv) Glu or Thr.

49. The polypeptide of any one of claims 43 to 48, wherein X₃ is selected from: i) Arg, Leu, lie, His, Glu, Trp, Ser, Ala, Val, Tyr, Asp, or Met; ii) Arg, Leu, Ile, His, Glu, or Trp; iii) Leu, Ile, His, Glu, Trp, Ser, Ala, Val, Tyr, Asp, or Met; or iv) Leu, Ile, His, Glu, or Trp.

50. The polypeptide of any one of claims 43 to 49, wherein X₄ is selected from: i) Phe, Tyr, Trp, Met, His, Lys, or Asn; ii) Phe, Tyr, or Trp; iii) Tyr, Trp, Met, His, Lys, or Asn; or iv) Trp or Tyr.

51. The polypeptide of any one of claims 43 to 50, wherein X₅ is selected from: i) His, Thr, Ser, or Gly; ii) His or Thr: iii) Thr, Ser, or Gly; or iv) Thr.

52. The polypeptide of any one of claims 43 to 51, wherein X₆ is selected from: i) His, Arg or Lys; ii) His or Arg; iii) Arg or Lys; or iv) Arg.

53. The polypeptide of any one of claims 43 to 52, wherein X₇ is selected from: i) Met, Glu or Asp; ii) Met or Glu; iii) Glu or Asp; or iv) Glu.

54. The polypeptide of any one of claims 43 to 53, wherein X₈ is selected from: i) Lys or Gly; ii) Lys; or iii) Gly.

55. The polypeptide of any one of claims 43 to 54, wherein X₅-X₆-X₇ is selected from the group consisting of: Thr-Arg-Glu; Thr-Arg-Asp; Thr-Lys-Glu; Thr-Lys-Asp; Ser-Arg-Glu; and Gly-Arg-Asp.

56. The polypeptide of any one of claims 43 to 55, wherein the polypeptide comprises from two to twenty independently selected mutant SPINK9 domains; and each mutant SPINK9 domain comprises an independently selected amino acid sequence of Formula I or SEQ ID NOS: 4-23.

57. The polypeptide of any one of claims 43 to 55, wherein the polypeptide comprises a first and a second independently selected mutant SPINK9 domain; and each of the first and the second mutant SPINK9 domains comprise an independently selected amino acid sequence of Formula I or SEQ ID NOS: 4-23.

58. The polypeptide of claim 57, wherein the first and the second mutant SPINK9 domains are identical.

59. The polypeptide of claim 57, wherein the first and the second mutant SPINK9 domains are not identical.

60. The polypeptide of any one of claims 43 to 55, wherein the polypeptide comprises a first, a second, and a third independently selected mutant SPINK9 domain; and each of the first, the second, and the third mutant SPINK9 domains comprise an independently selected amino acid sequence of Formula I or SEQ ID NOS: 4-23.

61. The polypeptide of claim 60, wherein each of said mutant SPINK9 domains are identical.

62. The polypeptide of claim 60, wherein each of said mutant SPINK9 domains are not identical.

63. The polypeptide of any one of claims 43 to 55, wherein the polypeptide comprises a first polypeptide chain and a second polypeptide chain linked through a Fc portion, wherein the first polypeptide chain comprises a first and a second mutant SPINK9 domain and the second polypeptide chain comprises a third and a fourth mutant SPINK9 domain and each of the first, second, third, and fourth mutant SPINK9 domains comprise an independently selected amino acid sequence of Formula I or SEQ ID NOS: 4-23.

64. The polypeptide of claim 63, wherein each of said mutant SPINK9 domains are identical.

65. The polypeptide of claim 64, wherein each of said mutant SPINK9 domains are not identical.

66. The polypeptide of any one of claims 56 to 65, wherein adjacent mutant SPINK9 domains are linked by direct fusion, a linker sequence, or any combination thereof.

67. The polypeptide of claim 66, wherein at least one mutant SPINK9 domain is linked to an adjacent mutant SPINK9 domain by a linker sequence.

68. The polypeptide of claim 66, wherein at least one mutant SPINK9 domain is linked to an adjacent mutant SPINK9 domain by direct fusion.

69. The polypeptide of any one of claims 66 to 68, wherein the linker sequence comprises a cleavage site.

70. The polypeptide of claim 69, wherein the cleavage site is recognized by a subtilisin-like proprotein convertase.

71. The polypeptide of any one of claims 43 to 55, wherein the polypeptide comprises a first polypeptide chain and a second polypeptide chain linked through a Fc portion, wherein the first polypeptide chain contains a first mutant SPINK9 domain and the second polypeptide chain contains a second mutant SPINK9 domain and each of the first and second mutant SPINK9 domains comprise an independently selected amino acid sequence of Formula I or SEQ ID NOS: 4-23.

72. The polypeptide of claim 71, wherein the first and the second mutant SPINK9 domains are identical.

73. The polypeptide of claim 71, wherein the first and the second mutant SPINK9 domains are not identical.

74. A polypeptide, comprising at least one mutant SPINK9 domain, wherein said at least one mutant SPINK9 domain comprises an amino acid sequence represented by Formula IV; X₉ X₁₀ X₁₁ Ala Lys Gln Thr Lys Gln Met Val Asp Cys Ser His Tyr Lys Lys Leu Pro Pro X₁ Gln X₂ X₃ X₄ Cys X₅ X₆ X₇ Tyr Asp Pro Ile Cys Gly Ser Asp Gly Lys Thr Tyr X₈ Asn Asp Cys Phe Phe Cys Ser Lys Val Lys Lys Thr Asp Gly Thr Leu Lys Phe Val His Phe Gly Lys Cys (SEQ ID NO: 136);
provided that the amino acid sequence represented by Formula IV is not SEQ ID NO: 2.

75. The polypeptide of claim 74, wherein the polypeptide binds to and inhibits an active kallikrein-related peptidase.

76. The polypeptide of claim 74 or 75, wherein the polypeptide inhibits the activity of kallikrein-related peptidase 5 (KLK5).

77. The polypeptide of claim 76, wherein the inhibition is selective for or specific to KLK5.

78. The polypeptide of any one of claims 74 to 67, wherein X₁ is selected from: i) Gly, Glu, Met, Gln, Ala, Leu, Val, Asp, Asn, Ser, Lys, or His; ii) Gly, Glu, or Met; iii) Glu, Met, Gln, Ala, Leu, Val, Asp, Asn, Ser, Lys, or His; or iv) Glu or Met.

79. The polypeptide of any one of claims 74 to 78, wherein X₂ is selected from: i) Gln, Glu, Thr, Val, Leu, Ala, Ile, Phe, Ser, or Tyr; ii) Gln, Glu, or Thr; iii) Glu, Thr, Val, Leu, Ala, lie, Phe, Ser, or Tyr; or iv) Glu or Thr.

80. The polypeptide of any one of claims 74 to 79, wherein X₃ is selected from: i) Arg, Leu, lie, His, Glu, Trp, Ser, Ala, Val, Tyr, Asp, or Met; ii) Arg, Leu, Ile, His, Glu, or Trp; iii) Leu, Ile, His, Glu, Trp, Ser, Ala, Val, Tyr, Asp, or Met; or iv) Leu, Ile, His, Glu, or Trp.

81. The polypeptide of any one of claims 74 to 80, wherein X₄ is selected from: i) Phe, Tyr, Trp, Met, His, Lys, or Asn; ii) Phe, Tyr, or Trp; iii) Tyr, Trp, Met, His, Lys, or Asn; or iv) Trp or Tyr.

82. The polypeptide of any one of claims 74 to 81, wherein X₅ is selected from: i) His, Thr, Ser, or Gly; ii) His or Thr: iii) Thr, Ser, or Gly; or iv) Thr.

83. The polypeptide of any one of claims 74 to 82, wherein X₆ is selected from: i) His, Arg or Lys; ii) His or Arg; iii) Arg or Lys; or iv) Arg.

84. The polypeptide of any one of claims 74 to 83, wherein X₇ is selected from: i) Met, Glu or Asp; ii) Met or Glu; iii) Glu or Asp; or iv) Glu.

85. The polypeptide of any one of claims 74 to 84, wherein X₈ is selected from: i) Lys or Gly; ii) Lys; or iii) Gly.

86. The polypeptide of any one of claims 74 to 85, wherein X₉ is selected from: i) absent; or ii) Ile.

87. The polypeptide of any one of claims 74 to 86, wherein X₁₀ is selected from: i) absent; or ii) Glu.

88. The polypeptide of any one of claims 74 to 87, wherein X₁₁ is selected from: i) absent; or ii) Cys.

89. The polypeptide of any one of claims 74 to 88, wherein X₅-X₆-X₇ is selected from the group consisting of: Thr-Arg-Glu; Thr-Arg-Asp; Thr-Lys-Glu; Thr-Lys-Asp; Ser-Arg-Glu; and Gly-Arg-Asp.

90. The polypeptide of any one of claims 74 to 89, wherein the polypeptide comprises from two to twenty independently selected mutant SPINK9 domains; and each mutant SPINK9 domain comprises an independently selected amino acid sequence of Formula IV or SEQ ID NOS: 115-134.

91. The polypeptide of any one of claims 74 to 89, wherein the polypeptide comprises a first and a second independently selected mutant SPINK9 domain; and each of the first and the second mutant SPINK9 domains comprise an independently selected amino acid sequence Formula IV or SEQ ID NOS: 115-134.

92. The polypeptide of claim 91, wherein the first and the second mutant SPINK9 domains are identical.

93. The polypeptide of claim 91, wherein the first and the second mutant SPINK9 domains are not identical.

94. The polypeptide of any one of claims 74 to 89, wherein the polypeptide comprises a first, a second, and a third independently selected mutant SPINK9 domain; and each of the first, the second, and the third mutant SPINK9 domains comprise an independently selected amino acid sequence of Formula IV or SEQ ID NOS: 115-134.

95. The polypeptide of claim 94, wherein each of said mutant SPINK9 domains are identical.

96. The polypeptide of claim 94, wherein each of said mutant SPINK9 domains are not identical.

97. The polypeptide of any one of claims 74 to 89, wherein the polypeptide comprises a first polypeptide chain and a second polypeptide chain linked through a Fc portion, wherein the first polypeptide chain comprises a first and a second mutant SPINK9 domain and the second polypeptide chain comprises a third and a fourth mutant SPINK9 domain and each of the first, second, third, and fourth mutant SPINK9 domains comprise an independently selected amino acid sequence of Formula IV or SEQ ID NOS: 115-134.

98. The polypeptide of claim 97, wherein each of said mutant SPINK9 domains are identical.

99. The polypeptide of claim 97, wherein each of said mutant SPINK9 domains are not identical.

100. The polypeptide of any one of claims 90 to 99, wherein adjacent mutant SPINK9 domains are linked by direct fusion, a linker sequence, or any combination thereof.

101. The polypeptide of claim 101, wherein at least one mutant SPINK9 domain is linked to an adjacent mutant SPINK9 domain by a linker sequence.

102. The polypeptide of claim 101, wherein at least one mutant SPINK9 domain is linked to an adjacent mutant SPINK9 domain by direct fusion.

103. The polypeptide of any one of claims 100 to 102, wherein the linker sequence comprises a cleavage site.

104. The polypeptide of claim 103, wherein the cleavage site is recognized by a subtilisin-like proprotein convertase.

105. The polypeptide of any one of claims 74 to 89, wherein the polypeptide comprises a first polypeptide chain and a second polypeptide chain linked through a Fc portion, wherein the first polypeptide chain contains a first mutant SPINK9 domain and the second polypeptide chain contains a second mutant SPINK9 domain and each of the first and second mutant SPINK9 domains comprise an independently selected amino acid sequence of Formula IV or SEQ ID NOS: 115-134.

106. The polypeptide of claim 105, wherein the first and the second mutant SPINK9 domains are identical.

107. The polypeptide of claim 105, wherein the first and the second mutant SPINK9 domains are not identical.

108. The polypeptide of any one of claims 1 to 107, further comprising a signal sequence.

109. The polypeptide of claim 108, wherein the signal sequence is linked to the amino (N)-terminus of the polypeptide by direct fusion, or by a linker sequence.

110. The polypeptide of claim 108, wherein the signal sequence is linked to the carboxy (C)-terminus of the polypeptide by direct fusion, or by a linker sequence.

111. The polypeptide of any one of claims 1 to 110, further comprising an immunoglobulin Fc region or a functional fragment thereof.

112. The polypeptide of claim 111, wherein the immunoglobulin Fc region or functional fragment thereof comprises an amino acid sequence selected from the group consisting of SEQ ID NOS: 39-54,

113. The polypeptide of claim 111, wherein the immunoglobulin Fc region or functional fragment thereof comprises an amino acid sequence of Formula I:
(SEQ ID NO: 24)
Glu Pro Lys Ser Cys Asp Lys Thr His Thr Cys Pro X₁
Cys Pro Ala Pro X₂ X₃ X₄ Gly X₅ Pro Ser Val Phe Leu
Phe Pro Pro Lys Pro Lys Asp Thr Leu X₆ Ile X₇ Arg X₈
Pro Glu Val Thr Cys Val Val X₉ Asp Val Ser His Glu
Asp Pro Glu Val Lys Phe Asn Trp Tyr Val Asp Gly Val
Glu Val His Asn Ala Lys Thr Lys Pro Arg Glu Glu Gln
Tyr X₁₀ Ser Thr Tyr Arg Val Val Ser Val Leu Thr Val
X₁₁ His X₁₂ Asp Trp Leu Asn Gly Lys Glu Tyr Lys Cys
Lys Val Ser Asn Lys X₁₃ Leu Pro X₁₄ X₁₅ Ile Glu Lys
Thr Ile Ser Lys Ala Lys Gly Gln Pro Arg Glu Pro Gln
Val Tyr Thr Leu Pro Pro Ser Arg Glu Glu Met Thr Lys
Asn Gln Val Ser Leu Thr Cys Leu Val Lys Gly Phe Tyr
Pro Ser Asp Ile Ala Val Glu Trp Glu Ser Asn Gly Gln
Pro Glu Asn Asn Tyr Lys Thr Thr Pro Pro Val Leu Asp
Ser Asp Gly Ser Phe Phe Leu Tyr Ser Lys Leu Thr Val
Asp Lys Ser Arg Trp Gln Gln Gly Asn Val Phe Ser Cys
Ser Val X₁₆ His Glu Ala Leu X₁₇ X₁₈ His Tyr Thr Gln
Lys Ser Leu Ser Leu Ser Pro Gly Lys;
wherein: X₁ is Pro or Ser; X₂ is Glu or Pro; X₃ and X₄ are each independently Lue or Ala; X₅ is Gly or Ala; X₆ is Met or Tyr; X₇ is Ser of Thr; X₈ is Thr or Glu; X₉ is Val or Glu; X₁₀ is Asn or Ala; X₁₁ is Leu or Asp; X₁₂ is Gln or His; X₁₃ is Ala or Gly; X₁₄ is Ala or Ser; X₁₅ is Pro or Ser; X₁₆ is Met or Leu; X₁₇ is His or Lys; and X₁₈ is Asn, Ser, or Phe.

114. The polypeptide of any one of claims 111 to 113, wherein the immunoglobulin Fc region or functional fragment thereof is linked to a mutant SPINK9 domain by direct fusion, or by a linker sequence.

115. The polypeptide of any one of claims 1 to 3, wherein the polypeptide comprises an amino acid sequence selected from the group consisting of SEQ ID NOS: 55-114.

116. The polypeptide of any one of claims 1 to 3, wherein the polypeptide comprises an amino acid sequence selected from the group consisting of SEQ ID NOS: 55-114, wherein amino acids 1-3 of SEQ ID NOS: 55-114 are deleted.

117. The polypeptide of any one of claims 1 to 3, wherein the polypeptide consists essentially of an amino acid sequence selected from the group consisting of SEQ ID NOS: 55-114.

118. The polypeptide of any one of claims 1 to 3, wherein the polypeptide consisting essentially of an amino acid sequence selected from the group consisting of SEQ ID NOS: 55-114, wherein amino acids 1-3 of SEQ ID NOS: 55-114 are deleted.

119. A nucleic acid, encoding the polypeptide of any one of claims 1-118.

120. A vector, comprising the nucleic acid of claim 119.

121. A host cell, comprising the nucleic acid of claim 119 or the vector of claim 120.

122. A method of producing a polypeptide comprising at least one mutant SPINK9 domain, said method comprising expressing the polypeptide in the host cell of claim 121, and isolating the polypeptide.

123. A composition, comprising the polypeptide of any one of claims 1 to 118, the nucleic acid according to claim 119, the vector according to claim 120, or the host cell according to claim 121.

124. A pharmaceutical composition, comprising a polypeptide of any one of claims 1 to 118, or a pharmaceutically acceptable salt thereof; and a pharmaceutically acceptable carrier.

125. A method of treating or preventing a disease or condition characterized by aberrant kallikrein activity, comprising administering to a subject in need thereof a therapeutically effective amount of a polypeptide of any one of claims 1 to 105, or a pharmaceutically acceptable salt thereof.

126. The method of claim 125, wherein the disease or condition is characterized by aberrant kallikrein-related peptidase 5 (KLK5) activity.

127. The method of claim 125 or 126, wherein the disease or condition is a skin disease.

128. The method of claim 125 or 126, wherein the disease or condition is Netherton Syndrome.

129. The method of claim 127, wherein the skin disease is eczema, atopic eczema, atopic dermatitis, autosomal recessive ichthyosis, ichthyosiform erythroderma, psoriasis, UV-induced skin injury, rosacea, or a skin infection.

130. The method of claim 125 or 126, wherein the disease or condition is selected from the group consisting of hypersensitivity of the immune system (atopy), hyper IgE syndrome, allergies (including allergies to food and airborne agents), asthma, allergic asthma, chronic inflammation, rhinitis, conjunctivitis, angioedema, eosinophilia, eosinophilic esophagitis, growth delay, failure to thrive, trichorrhexis invaginata (TI), respiratory tract infections, systemic infections, and gastrointestinal disorders.

131. The method of claim 125 or 126, wherein the disease or condition is cancer.

132. The method of claim 131, wherein the cancer is selected from the group consisting of ovarian cancer, uterine cancer, colorectal cancer, bladder urothelial cancer, oral squamous cell carcinoma, breast cancer, prostate cancer, bladder cancer, cervical cancer, melanoma, head and neck cancer glioma, glioblastoma multiforme, and neuroblastoma.
